(19) United States
(12) Patent Application Publication
(10) Pub. No.: US 2026/0248924 A1
(43) Pub. Date: Aug. 27, 2026

(54) MITOCHONDRIA-TARGETED AGENTS FOR DISEASE PREVENTION AND TREATMENT

(71) Applicant: THE METHODIST HOSPITAL, Houston, TX (US)

(72) Inventors: Ming YOU, Houston, TX (US); Jiaheng LI, Houston, TX (US); Yangxiong LI, Houston, TX (US)

(21) Appl. No.: 19/163,181

(22) PCT Filed: Mar. 7, 2024
(86) PCT No.: PCT/US2024/018797

§ 371 Date: Sep. 8, 2025

Related U.S. Application Data
(60) Provisional application No. 63/450,795, filed on Mar. 8, 2023.

(51) Int. Cl. A61K 47/54; A61P 35/00

(52) U.S. Cl. CPC A61K 47/548; A61P 35/00

(57) ABSTRACT

Described herein are compounds of formula I, compositions and methods of using thereof.

### CROSS REFERENCE TO RELATED APPLICATIONS

This application claims the benefit of priority to U.S. Provisional Application No. 63/450,795, filed Mar. 8, 2023, which is incorporated by reference herein in its entirety.

### BACKGROUND

Mitochondria, the central site for cellular energy production, have important functions in cell survival and death. There is a significant role for mitochondrial metabolism in promoting cancer development and progression, making mitochondria a promising new target for cancer prevention/treatment. Conjugating delocalized lipophilic cations, such as triphenylphosphonium cation (TPP+), to compounds of interest is an effective approach for mitochondrial targeting. The hyperpolarized tumor cell membrane and mitochondrial membrane potential allow for selective accumulation of TPP+ conjugates in tumor cell mitochondria versus those in normal cells. This could enhance direct killing of precancerous, dysplastic, and tumor cells while minimizing potential toxicities to normal cells.

There is a need for effective cancer preventive/therapeutic agents, such as mitochondria-targeted drugs, that can prevent/treat cancer and prolong survival. The compositions and methods disclosed herein address these and other needs.

### SUMMARY

Disclosed herein are compounds, compositions, methods for making and using such compounds and compositions. In further aspects, disclosed herein are compounds of formula I

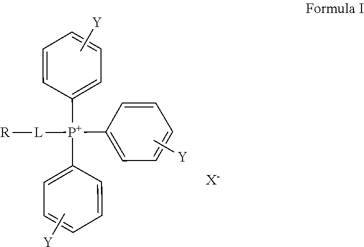

or pharmaceutically acceptable salts, prodrugs, or derivatives thereof,
   wherein:
L is selected from an unsubstituted or substituted C₁-C₂₀ alkyl, unsubstituted or substituted C₂-C₂₀ alkenyl, unsubstituted or substituted C₁-C₂₀ alkynyl, unsubstituted or substituted aryl, unsubstituted or substituted cycloalkyl, or unsubstituted or substituted heterocycle;
Y are each independently selected from H, —OR^{a}, haloalkyl, or halogen;
R^{a} is an unsubstituted or substituted alkyl, or haloalkyl;
X is an anion;
R is a substituted or unsubstituted alkyl, alkoxy, cycloalkyl, aryl, heteroaryl, heterocycloalkyl, ether, ester, amine, amide, or sulfide.

In some embodiments, R can be

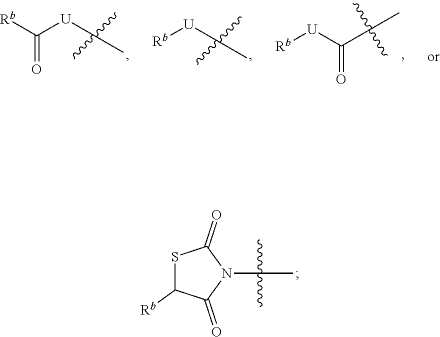

wherein
R^{b} is substituted or unsubstituted alkyl, cycloalkyl, cycloalkenyl, heterocycloalkyl, heterocycloalkenyl, aryl, alkylaryl, or heteroaryl;
U is O, S, —NH, or —CR^{c}R^{d}; and
R^{c} and R^{d} are each independently —H or alkyl.

In some embodiments, R can be selected from:

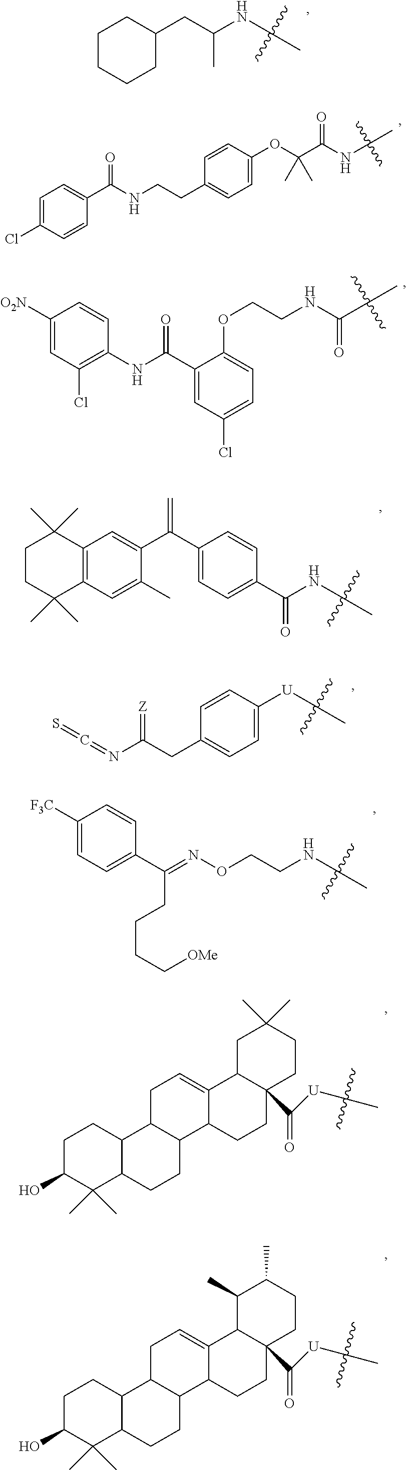

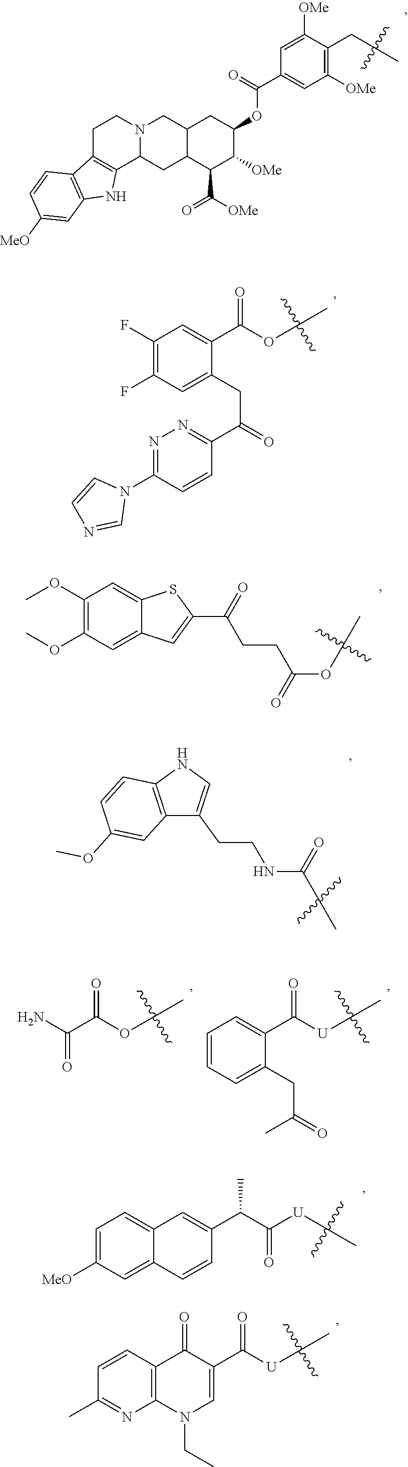

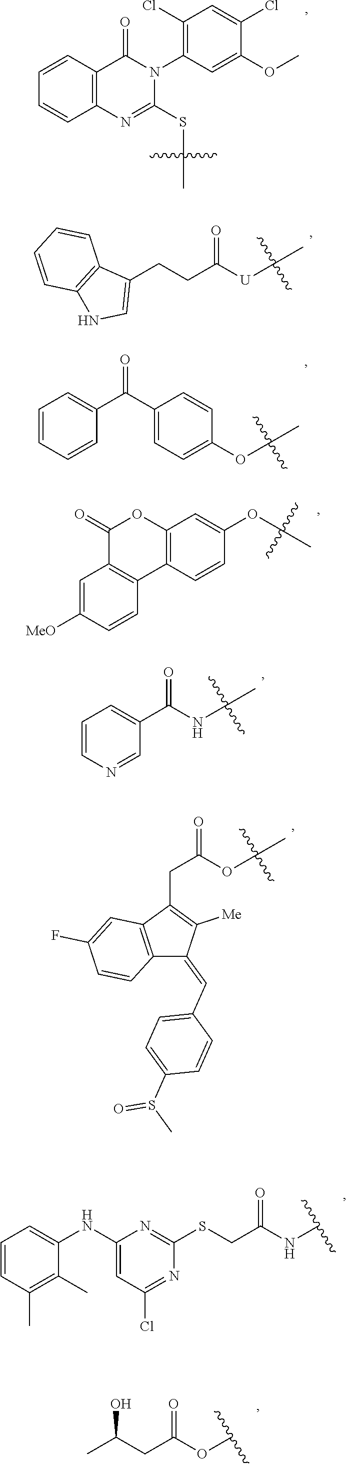

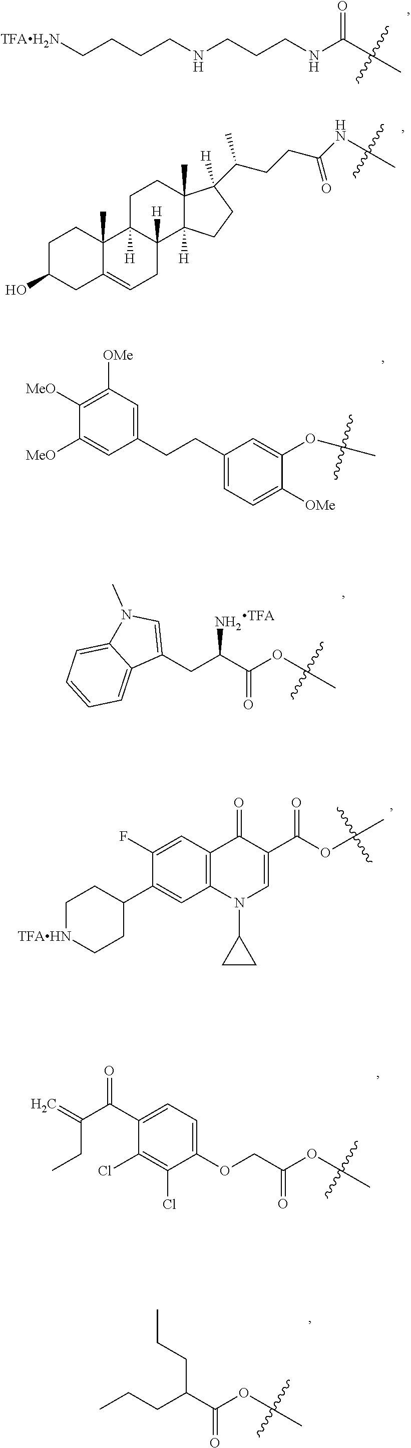

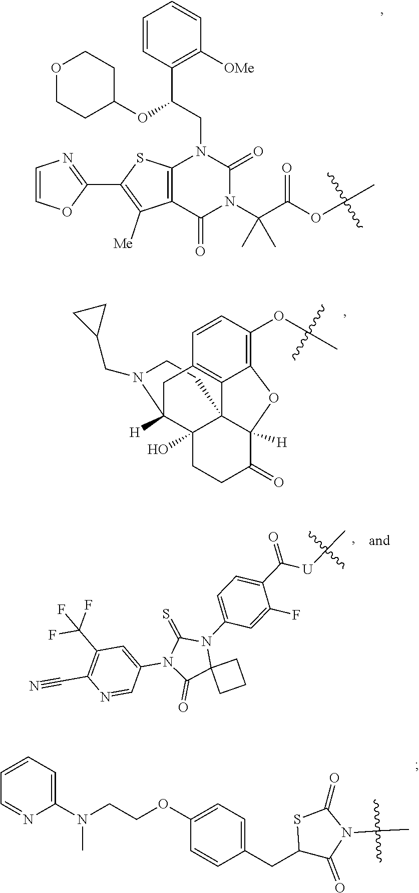

U is O, S, or NH; and
Z is —H₂ or —CH₂CH₂.

Described herein are also pharmaceutical compositions comprising an effective amount of the compound described herein and a pharmaceutically acceptable carrier.

Described herein are also methods for treating or preventing cancer, the methods including administering to a subject in need thereof an effective amount of the pharmaceutical composition described herein or an effective amount of a compound described herein.

The details of one or more embodiments of the disclosure are set forth in the accompanying drawings and the description below. Other features, objects, and advantages of the disclosure will be apparent from the description and drawings, and from the claims.

### DESCRIPTION OF FIGURES

The accompanying figures, which are incorporated in and constitute a part of this specification, illustrate several aspects and together with the description serve to explain the principles of the invention.

FIG. 1 is a graph of lung cancer cell line confluence at different concentrations of Bezafibrate and mito-Bezfibrate.

FIG. 2 is a graph of H2030-BrM3 lung cancer cell line confluence at different concentrations of Bexarotene and mito-Bexarotene.

FIGS. 3A-3B are graphs of H2030-BrM3 (FIG. 3A) and LKR-13 (FIG. 3B) lung cancer cell lines confluence at different concentrations of phenethyl isothiocyanate (PEITC) and mito-PEITC.

FIG. 4 is a graph showing SR717 and mito-SR717 induced IFN secretion.

FIG. 5 is a graph of H2030-BrM3 lung cancer cell lines confluence at different concentrations of MSA-2 and mito-MSA-2.

FIG. 6 is a graph of H2030-BrM3 lung cancer cell lines confluence at different concentrations of melatonin and mito-melatonin.

FIG. 7 is a graph of H2030-BrM3 lung cancer cell lines confluence at different concentrations of oxamate and mito-oxamate.

FIG. 8 illustrates chemical structures and synthesis of Mito-PEITC. a.1. CS₂, Et₃N, EtOH; 2. Boc₂O, DMAP. b. Ph₃P⁺C₁₀H₂₀Br, K₂CO₃, DMF.

FIGS. 9A-9B show the effect of PEITC on B(a)P-induced lung tumorigenesis in A/J mice. (9A) Tumor multiplicity; (9B) Tumor load. n=10; ***P<0.001

FIGS. 10A-10D show selected measures of 35 total from the Modified Irwin Screen: Mito-PEITC toxicity. (10A) Measured body weight (squares, left axis) and rectal temperature (circles, right axis). (10B) Latency to fall measured after 8-week treatment. (10C&10D) Blood level of liver enzymes after 8-week treatment. n=5 FIGS. 11A-11C show graphs demonstrating prevention of lung cancer brain metastasis by PEITC and Mito-PEITC. (11A) Visualization of needle with use of high-resolution echocardiography. (111B) Bioluminescence-based imaging of the growth of brain metastases. Inset: LC-MS trace of Mito-PEITC (peak indicated by the arrow) detected in the brain tissue extract. (11C) Representative bioluminescence, GFP expression and H&E staining images of brains taken from control, PEITC and Mito-PEITC groups.

FIGS. 12A-12B show the accumulation of PEITC (12A) and Mito-PEITC (12B) in cell cytosol (CYTO) and mitochondria (MITO). H2030-BrM3 cells were treated for 24 h with the compounds (100 nM), subcellular fractions were isolated, compounds derivatized with 5% NH₃, and analyzed by LC-MS. Insets: representative LC-MS traces of the analytes.

FIGS. 13A-13H: Mito-PEITC shows stronger antiproliferative potency than PEITC and is nontoxic in mice. (13A) Chemical structures of PEITC and Mito-PEITC and synthesis of Mito-PEITC. a.1. CS₂, Et₃N, EtOH; 2. Boc₂O, DMAP. b. Ph₃P⁺(CH₂)₁₀Br, K₂CO₃, DMF. (13B) Effect of Mito-PEITC on the proliferation of H2030-BrM3, PC9-BrM3 and NHBE cells. (13C-13D) Concentration dependence of the effect of PEITC and Mito-PEITC on the proliferation of H2030-BrM3 (13C) and LKR-13 (D) LUAD cells. (E-F) Selected measures of toxicity from the 35 metrics of the modified Irwin Screen conducted on mice after daily administration with Mito-PEITC. Dose 1× corresponds to 4 μmol/kg. Data presented are average values ±S.D. (13E) Measured body weight (squares, left axis) and rectal temperature (circles, right axis). (13F) Latency to fall measured after 8-week treatment. The blood levels of AST (13G) and ALT (13H) liver enzymes after 8-week treatment (n=5 mice per group).

FIGS. 14A-14G Mito-PEITC shows a preventive effect in lung cancer models in mice. (14A-14B) Accumulation of PEITC (14A) and Mito-PEITC (14B) in cell cytosol (CYTO) and mitochondria (MITO). H2030-BrM3 cells were treated for 24 h with compounds (100 nM), subcellular fractions were isolated, compounds derivatized with 5% NH₃, and analyzed by LC-MS. Insets: representative LC-MS traces of the analytes. (14C) Prevention of lung cancer by PEITC and Mito-PEITC (4 μmol/kg each) in an orthotopic model. Upper panel, bioluminescence-based imaging; lower panel, BLI signal intensity of orthotopic tumors over time. (14D-14E) Prevention of lung cancer brain metastasis by PEITC and Mito-PEITC (4 μmol/kg each). (14D) bioluminescence-based imaging of brain metastases. Representative bioluminescence, GFP expression, and H&E staining images of brains taken from control, PEITC, and Mito-PEITC groups. (14E) BLI signal intensity of brain metastases monitored over time. (14F, 14G) Effect of PEITC (100 μmol/kg) and Mito-PEITC (4 μmol/kg) on VC-induced lung tumorigenesis in A/J mice after 20 weeks of administration. (14F) Tumor multiplicity; (14G) Tumor load. n=10 mice per group; ***P<0.001

FIGS. 15A-15H: Mito-PEITC inhibits activity of mitochondrial complexes I and III. (15A, 15C) selected Seahorse XF OCR traces representing mitochondrial complex I (15A) and complex III (15C) activity from cells treated with indicated concentration of PEITC or Mito-PEITC for 24 h. The injection points of inhibitors of complex I (rotenone, ROT) or complex III (antimycin A, AA) are indicated by the arrows. Inhibition concentration curves of complex I (15B) or complex III (15D) by PEITC and Mito-PEITC. (15E-15F) Effect of Mito-PEITC (100 nM, 24 h) on cellular ROS production, as measured by HPLC-based analyses of the oxidation products of hydroethidine (HE). (15E) HPLC traces; (15F) quantitative analyses of HE oxidation products. (15G-15H) Effect of Mito-PEITC (100 nM, 24 h) on the oxidation status of cytosolic (Prx1) and mitochondrial (Prx3) peroxiredoxins. (15G) Representative immunoblots showing the intensity of bands of the reduced, monomeric (red-Prx) and oxidized, dimeric (ox-Prx) forms of peroxiredoxins; (15H) quantitative analyses of the oxidation status of Prx1 and Prx3. **p<0.01

FIGS. 16A-16I. Role of STAT3 in the anti-cancer effects of PEITC and Mito-PEITC. (16A-16B) Effect of PEITC (20 μM) or Mito-PEITC (0.2 μM) on protein phosphorylation status in H2030-BrM3 cells, measured by receptor tyrosine kinase proteomic array (16A) and immunoblotting of identified protein targets (16B). (16C) Western blot showing knockout efficiency of STAT3 in H2030-BrM3 cells. (16D) Representative transwell invasion images from different treatment groups. (16E) STAT3 KO partially abrogates the anti-invasive effects of Mito-PEITC (0.2 μM, 24 h) in H2030-BrM3 cells. (16F-16H) Mito-PEITC (0.2 μM, 24 h) treatment upregulates AMPK (16F), downregulates STAT3 (16G), and upregulates apoptosis (16H) pathways in LKR-13 cells, as identified by RNA-seq analyses. (16I) Flow cytometric analyses of early- and late-apoptotic cells upon treatment of H2030-BrM3 cells with PEITC (20 μM) or Mito-PEITC (0.2 μM).

FIGS. 17A-17E. Direct reaction of Mito-PEITC with GSH. (17A) Mito-PEITC depletion and GSH adduct formation kinetics in the presence of 0.1 mM GSH. (17B) Equilibrium of Mito-PEITC-GSH adduct formation as a function of GSH concentration. (17C) Left: PicoGreen staining for the absence of (mtDNA) mitochondria in B16 ρ0 cells. Arrows indicate mitochondria fibrillar network. Right: Comparison of the mitochondrial respiratory function of B16 and B16 ρ0 cells. (17D-17E) mtDNA depletion abrogates the antiproliferative effects of Mito-PEITC (0.3 M, 48 h) in B16 ρ0 cells. (17D) Representative proliferation traces; (17E) Comparison of the cell confluency after 48-h treatment period. ***P<0.001 vs. parental cells. Error bars: SD.

FIGS. 18A-18L Single-cell clustering analysis based on the full scRNA-seq data and canonical marker analysis of VC-induced lung tumors. (18A-18I) Gene expression of canonical markers for T cells and B cells. (18J) Left: CD45+ cells annotated into various types of immune cells, (e.g., natural killer cells, macrophages, neutrophils). Right: The percentage changes of the detected CD45+ cell types in tumors from control (vehicle) and Mito-PEITC (4 μmol/kg)-treated mice. (18K) CD45− cells annotated into four cell types, (i.e., tumor cells, epithelial cells, endothelial cells, and fibroblasts). (18L) The percentage changes of the detected four CD45− cell types in tumors from control (vehicle) and Mito-PEITC (4 μmol/kg)-treated mice.

FIGS. 19A-19G Use of scRNA-seq to characterize CD8+ TIL subsets in VC-induced lung tumors in A/J mice. (19A-19D) Clustering of CD45+ immune cells. (19E) Heatmap showing gene expression of marker for the four tumor-infiltrating CD8+ T cell states in CD8+ TILs from mouse lung tumors. (19F) Clustering of intratumoral CD8+ T cells into the four indicated CD8 TIL subsets. (19G) Mito-PEITC treatment (4 μmol/kg) increased the proportion of the EM-like CD8+ TILs.

FIGS. 20A-20H Mito-PEITC treatment reduces G-MDSC percentage in VC-induced lung tumors in A/J mice. (20A-20F) General neutrophils were identified by co-expression of the indicated marker genes. (20G) Clustering analysis of cell populations between G-MDSC, M-MDSC, and normal neutrophils. (20H) The G-MDSC subset percentage was drastically decreased by Mito-PEITC treatment (4 μmol/kg) in mouse tumors relative to the control group.

FIGS. 21A-21I Mito-PEITC reshapes the tumor microenvironment by decreasing immune suppressive G-MDSC and Tregs, leading to increased antitumor T cell immunity in syngraft model. (21A-21E) Percentage of G-MDSCs (21A), Treg cells (21B), Granzyme B cells (21C), CD4+ TILs (21D), and CD8+ TTLs (21E) in cell populations isolated from tumors from control- and Mito-PEITC (4 μmol/kg)-treated mice. (21F-21G) Tumor burden as measured by tumor size (21F) and percent survival (21G) in mice treated with Mito-PEITC (4 μmol/kg) alone or with anti-CD4 or anti-CD8 monoclonal antibodies. Error bars depict SEM. (21H-21I) Tumor burden as measured by tumor size (21H) and percent survival (21I) in mice treated with Mito-PEITC (4 μmol/kg) or anti-PD-1 monoclonal antibodies alone or in combination. (* p≤0.05; ** p≤0.01; *** p≤0.001).

FIGS. 22A-22F. Local injection of Mito-PEITC eradicates established local tumors and induces systematic antitumor effects in brain and lung metastasis models. (22A) Experimental design and timeline. (22B) Bioluminescence imaging of lung metastases in representative SV129 mice. (22C) Quantification of bioluminescence imaging in Mito-PEITC-treated (4 μmol/kg) and control animals. (22D) Survival of SV129 mice treated with Mito-PEITC vs. control. (22E) Bioluminescence imaging of lung metastases in representative SV129 mice. (22F) Survival of SV129 mice treated with Mito-PEITC vs. control.

FIG. 23. Differential expression of metabolic pathways in tumor cells in VC-induced lung carcinogenesis model. Pathways (dots) are partitioned by Recon2 pathways and colored by the sign of their Cohen's d statistics. Image summarizes major metabolism pathway activity changes by Mito-PEITC (4 μmol/kg) treatment in VC-induced lung cancer cells.

FIG. 24. Differential expression of metabolic pathways in Cd8+ EM-like T cells. Reactions (dots) are partitioned by Recon2 pathways and colored by the sign of their Cohen's d statistic. The image summarizes major metabolism pathway activity changes by Mito-PEITC (4 μmol/kg) treatment in CD8+ EM-like T cells.

FIG. 25 shows a graph of cell proliferation assay.

FIG. 26 shows a graph of cell proliferation assay.

FIG. 27 shows a graph of cell proliferation assay.

FIG. 28 shows a graph of cell proliferation assay.

FIG. 29 shows a graph of cell proliferation assay.

FIG. 30 shows a graph of cell proliferation assay.

FIG. 31 shows a graph of cell proliferation assay.

FIG. 32 shows a graph of cell proliferation assay.

FIG. 33 shows a graph of cell proliferation assay.

FIG. 34 shows a graph of cell proliferation assay.

FIG. 35 shows a graph of cell proliferation assay.

FIG. 36 shows a graph of cell proliferation assay.

FIG. 37 shows a graph of cell proliferation assay.

FIG. 38 shows a graph of cell proliferation assay.

FIG. 39 shows a graph of cell proliferation assay.

FIG. 40 shows a graph of cell proliferation assay.

FIG. 41 shows a graph of cell proliferation assay.

FIG. 42 shows a graph of cell proliferation assay.

FIG. 43 shows a graph of cell proliferation assay.

FIG. 44 shows a graph of cell proliferation assay.

FIG. 45 shows a graph of cell proliferation assay.

FIG. 46 shows a graph of cell proliferation assay.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

“Administration” to a subject includes any route of introducing or delivering to a subject an agent. Administration can be carried out by any suitable route, including oral, topical, transcutaneous, transdermal, intra-joint, intra-arteriole, intradermal, intraventricular, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect.

Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed.

A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts. An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained.

When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods.

Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient.

The term “patient” refers to a subject under the treatment of a clinician, e.g., physician.

Administration of the therapeutic agents can be carried out at dosages and for periods of time effective for treatment of a subject. In some embodiments, the subject is a human.

Chemical Definitions Terms used herein will have their customary meaning in the art unless specified otherwise.

The organic moieties mentioned when defining variable positions within the general formulae described herein (e.g., the term “halogen”) are collective terms for the individual substituents encompassed by the organic moiety. Ph in Formula I refers to a phenyl group.

The prefix Cn-Cm preceding a group or moiety indicates, in each case, the possible number of carbon atoms in the group or moiety that follows.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, heteroatoms present in a compound or moiety, such as nitrogen, can have hydrogen substituents and/or any permissible substituents of organic compounds described herein which satisfy the valency of the heteroatom. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound (e.g., a compound that does not spontaneously undergo transformation such as by rearrangement, cyclization, elimination, etc.

The term “optionally substituted,” as used herein, means that substitution with an additional group is optional and therefore it is possible for the designated atom to be unsubstituted. Thus, by use of the term “optionally substituted” the disclosure includes examples where the group is substituted and examples where it is not.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, and 1-ethyl-2-methyl-propyl. Alkyl substituents may be unsubstituted or substituted with one or more chemical moieties. The alkyl group can be substituted with one or more groups including, but not limited to, hydroxy, halogen, acyl, alkyl, alkoxy, alkenyl, alkynyl, aryl, heteroaryl, acyl, aldehyde, amino, carboxylic acid, ester, ether, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied. The alkyl group can also include one or more heteroatoms (e.g., from one to three heteroatoms) incorporated within the hydrocarbon moiety. Examples of heteroatoms include, but are not limited to, nitrogen, oxygen, sulfur, and phosphorus.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. The term “alkylthiol” specifically refers to an alkyl group that is substituted with one or more thiol groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkenyl groups are intended.

Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, halogenated alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, thiosulfonate (e.g., —SSO₂Ra), or thiol, as described below.

As used herein, the term “aryl,” as well as derivative terms such as aryloxy, refers to groups that include a monovalent aromatic carbocyclic group of from 3 to 20 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphthyl, phenylcyclopropyl, and indanyl. In some embodiments, the aryl group can be a phenyl, indanyl or naphthyl group. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl substituents may be unsubstituted or substituted with one or more chemical moieties.

Examples of suitable substituents include, for example, alkyl, halogenated alkyl, alkoxy, alkenyl, alkynyl, aryl, heteroaryl, acyl, aldehyde, amino, carboxylic acid, cycloalkyl, ester, ether, halide, hydroxy, ketone, nitro, silyl, sulfo-oxo, sulfonyl, sulfone, sulfoxide, or thiol as described herein.

The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. cycloalkyl groups can include a single non-aromatic carbon-based ring or multiple condensed non-aromatic carbon-based rings. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acyl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups.

Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “acyl” as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a short hand notation for C═O.

As used herein, the term “alkoxy” refers to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O−.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

As used herein, Me refers to a methyl group; OMe refers to a methoxy group; and i-Pr refers to an isopropyl group.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context can include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context can be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Compounds

Described herein are compounds of formula I

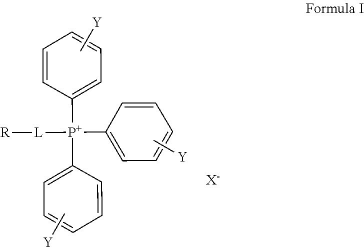

or pharmaceutically acceptable salts, prodrugs, or derivatives thereof,
wherein:
L is selected from an unsubstituted or substituted C₁-C₂₀ alkyl, unsubstituted or substituted C₂-C₂₀ alkenyl, unsubstituted or substituted C₁-C₂₀ alkynyl, unsubstituted or substituted aryl, unsubstituted or substituted cycloalkyl, or unsubstituted or substituted heterocycle;
Y are each independently selected from H, —OR^{a}, haloalkyl, or halogen;
R^{a} is an unsubstituted or substituted alkyl, or haloalkyl; and
X is an anion;
R is a substituted or unsubstituted alkyl, alkoxy, cycloalkyl, aryl, heteroaryl, heterocycloalkyl, ether, ester, amine, amide, or sulfide.

In some embodiments R can be

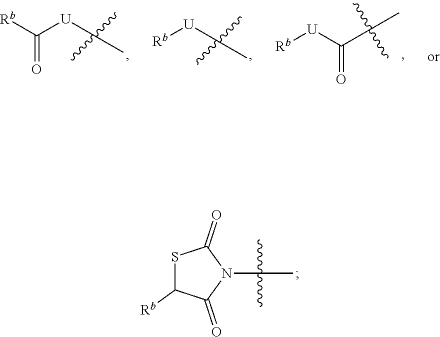

wherein
R^{b} is substituted or unsubstituted alkyl, cycloalkyl, cycloalkenyl, heterocycloalkyl, heterocycloalkenyl, aryl, alkylaryl, or heteroaryl;
U is O, S, —NH, or —CR^{c}R^{d}; and
R^{c} and R^{d} are each independently —H or alkyl.

In some embodiments, R can be selected from:

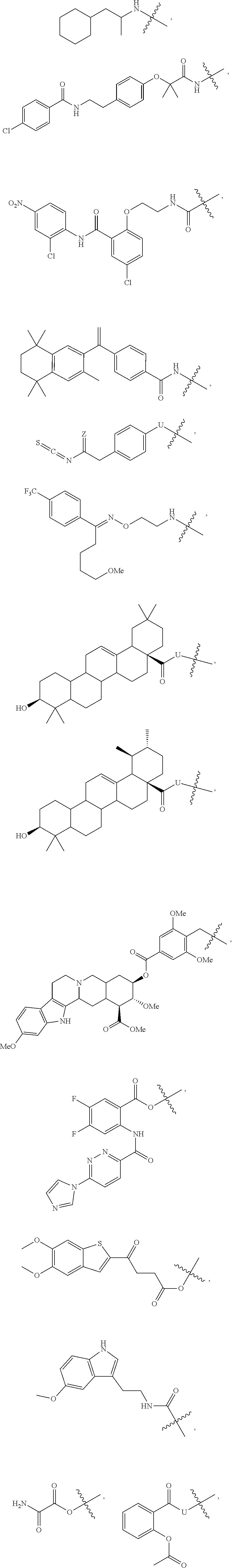

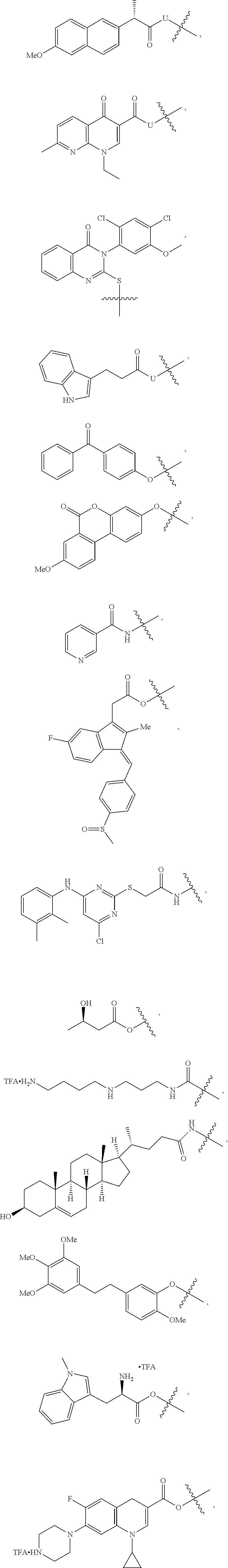

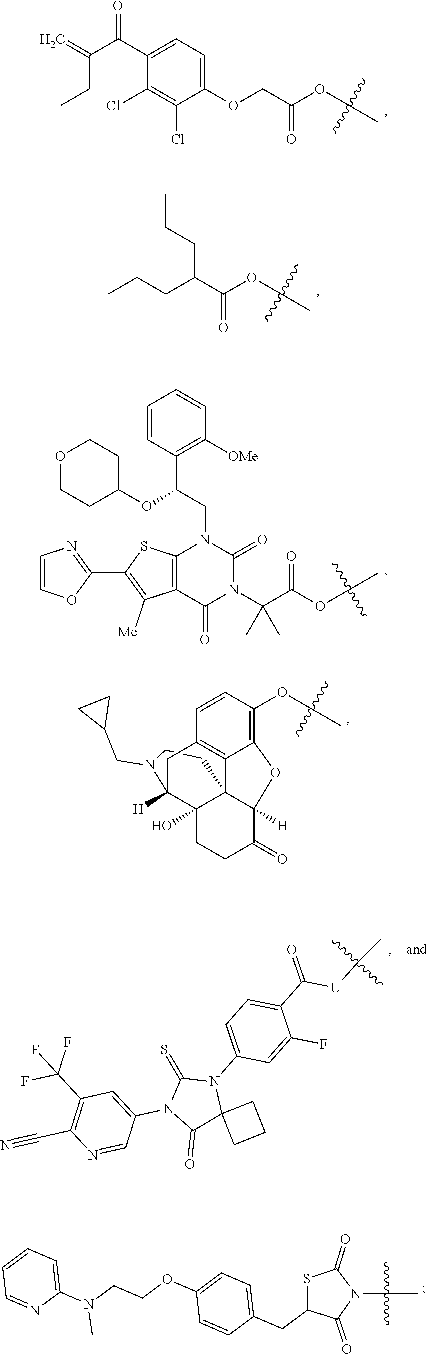

U is O, S, or NH; and
Z is —H₂ or —CH₂CH₂.

In some embodiments, described are compounds of formula I

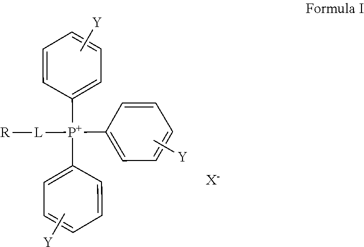

or pharmaceutically acceptable salts, prodrugs, or derivatives thereof,
wherein:
L is selected from an unsubstituted or substituted C₁-C₂₀ alkyl, unsubstituted or substituted C₂-C₂₀ alkenyl, unsubstituted or substituted C₁-C₂₀ alkynyl, unsubstituted or substituted aryl, unsubstituted or substituted cycloalkyl, or unsubstituted or substituted heterocycle;
Y are each independently selected from H, —OR^{a}, haloalkyl, or halogen;
R^{a} is an unsubstituted or substituted alkyl, or haloalkyl; and
X is an anion;
R is selected from:

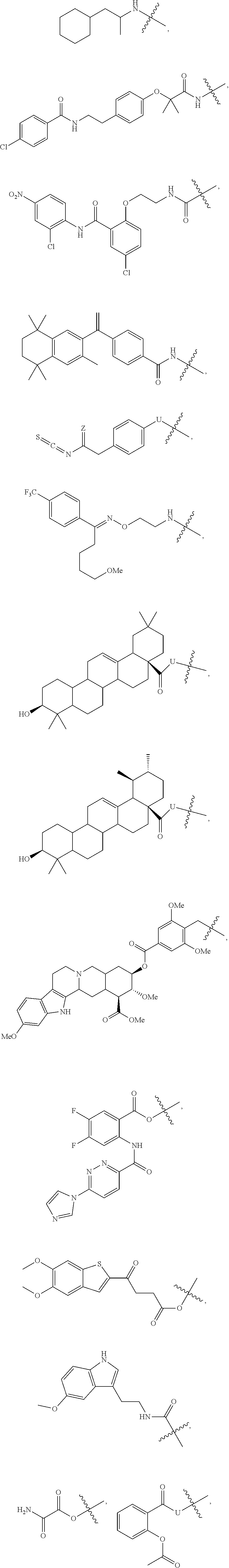

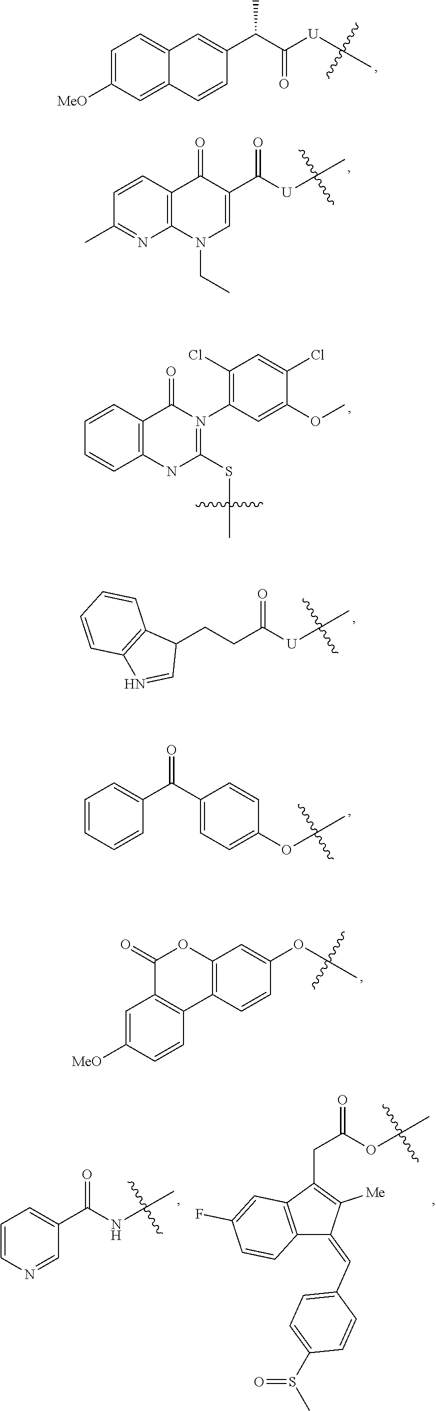

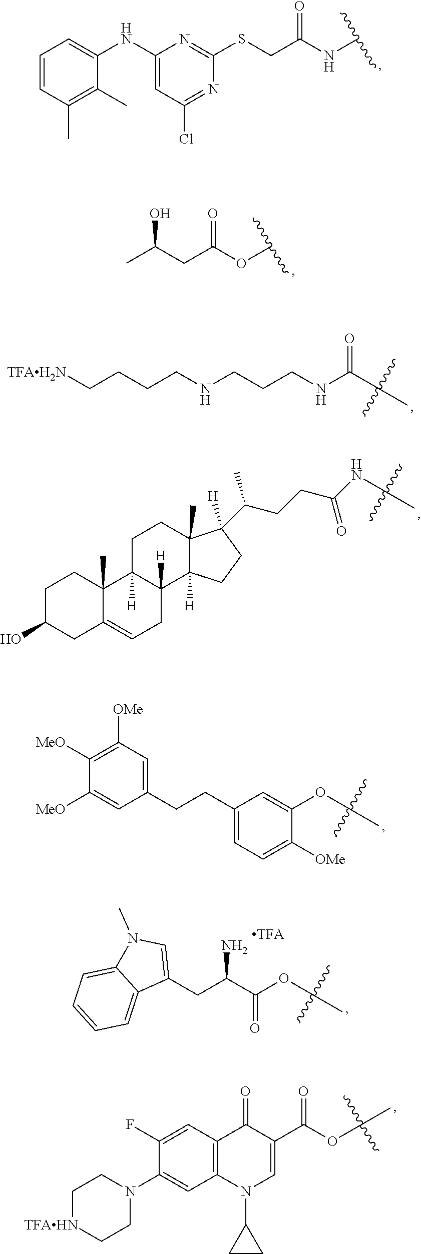

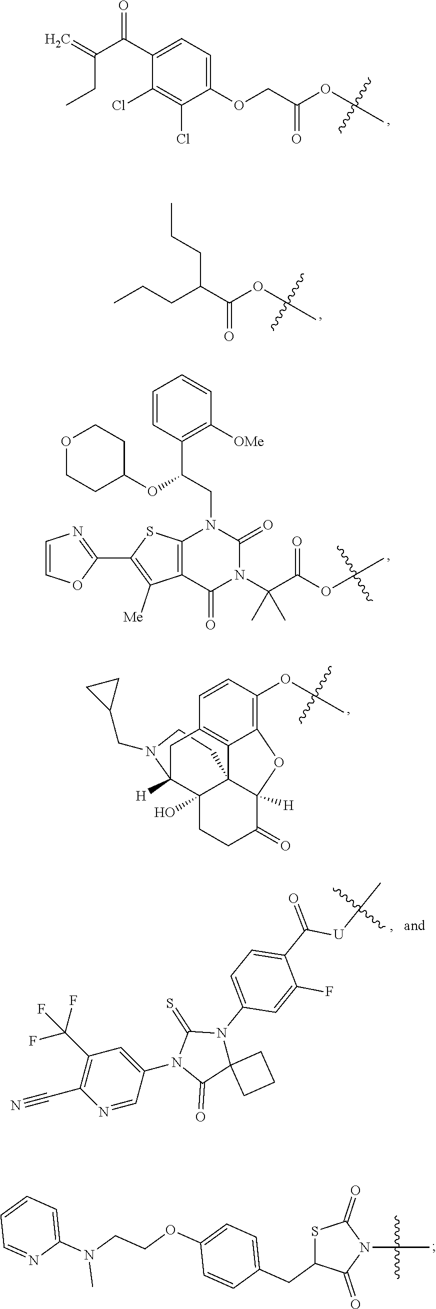

U is O, S, or NH; and
Z is —H₂ or —CH₂CH₂.

In some embodiments, the R can be

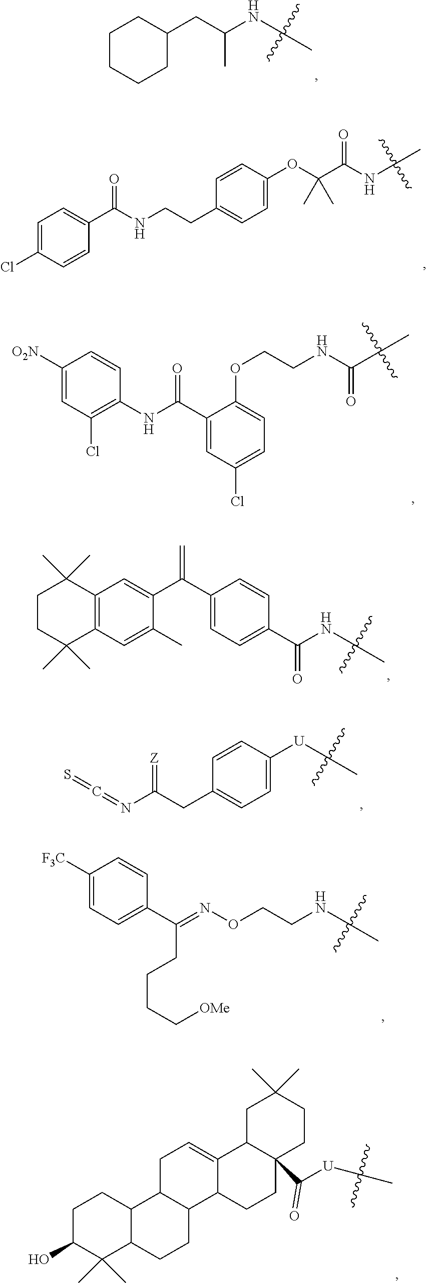

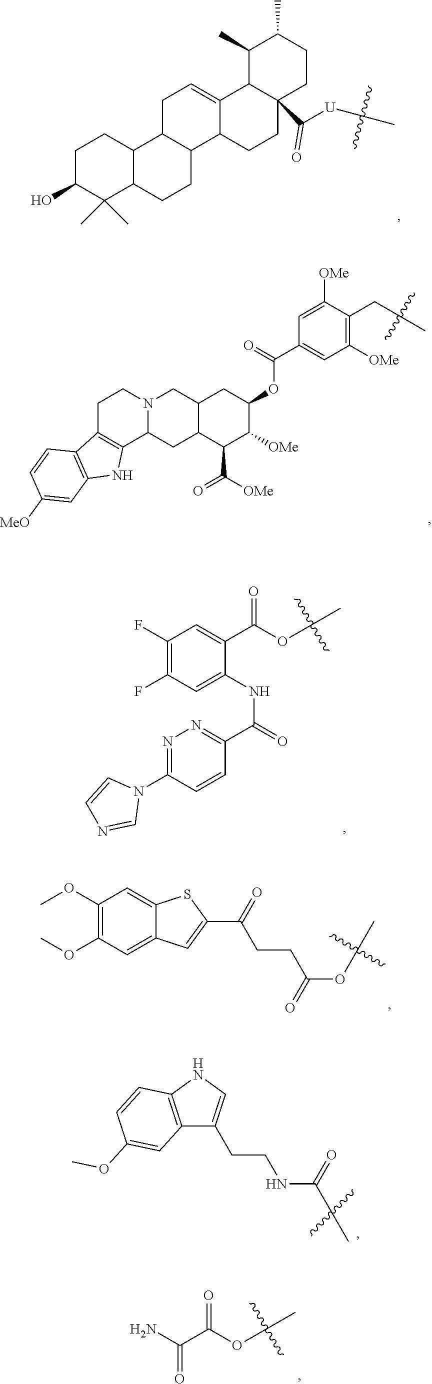

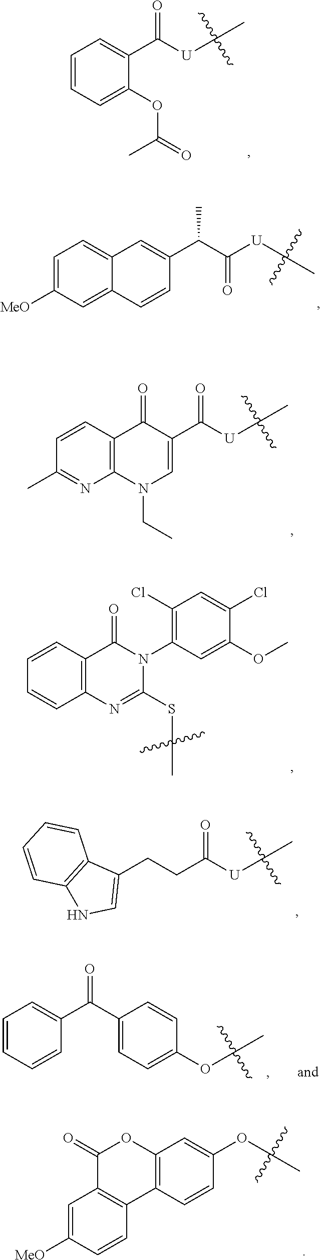

In some embodiments, the R can be

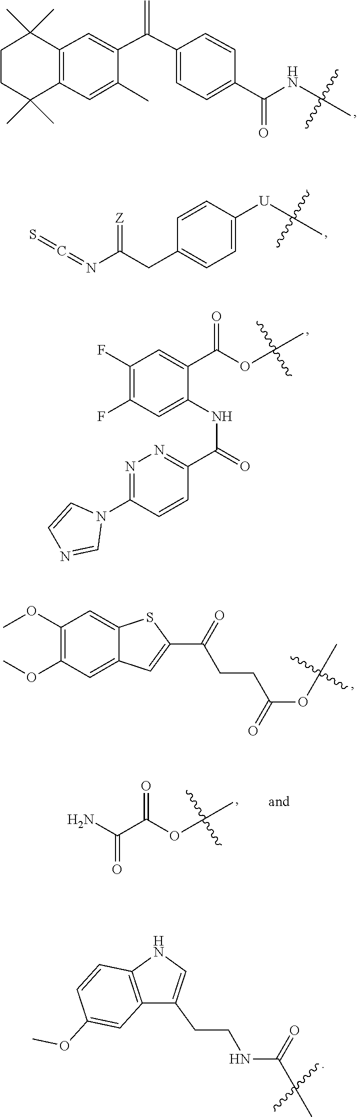

In some embodiments, X can be a halogen, trifluoroacetic acid, triflate, hexafluorophosphate or acetic acid. In some embodiments, X can be halogen.

In some embodiments, L can be phenyl. In some embodiments, L can be phenyl substituted C₁-C₂₀ alkyl. In some embodiments, L can be cycloalkyl substituted C1-C20 alkyl. In some embodiments, L can be poly(ethylene glycol) (PEG). In some embodiments, L can be a substituted heterocycle. In some embodiments, L can be substituted or unsubstituted C1-C20 alkyl. In some embodiments, L can be unsubstituted C1-C20 alkyl. In some embodiments, L can be C1, C2, C3, C4, C5, C6, C7, C8, C9, C10, C11, C12, C13, C14, C15, C16, C17, C18, C19, or C20 alkyl. In some embodiments, L can be C10 alkyl. In some embodiments, L can be C9 alkyl.

In some embodiments, Y can be a halogen. In some embodiments, Y can be Cl. In some embodiments, Y can be —OR^{a} and R^{a} can be an alkyl. In some embodiments, Y can be methoxy. In some embodiments, Y can be —OR^{a} and R^{a} can be an haloalkyl. In some embodiments, Y is trifluoromethoxy. In some embodiments, Y can be a haloalkyl. In some embodiments, Y can be trifluoromethyl. In some embodiments, Y can be H.

In some embodiments, Y can be located at an ortho, meta, or para position. In some embodiments, Y can be located at an ortho position. In some embodiments, Y can be located at a meta position. In some embodiments, Y can be located at a para position. In some embodiments, Y can be located at an ortho and meta positions. In some embodiments, Y can be located at an ortho and para positions. In some embodiments, Y can be located at a meta and para positions. In some embodiments, Y can be located at an ortho, meta, and para positions.

In some embodiments, U can be NH. In some embodiments, U can be O. In some embodiments, U can be S.

In some embodiments, R can be

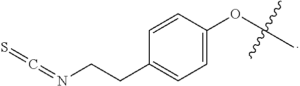 [SMILES: *Oc1ccc(CCN=C=S)cc1]

In some embodiments, the compound can be selected from:

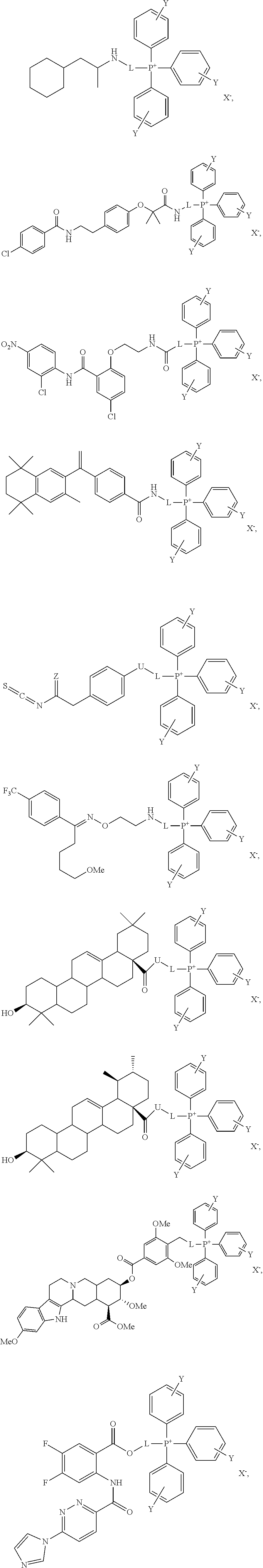

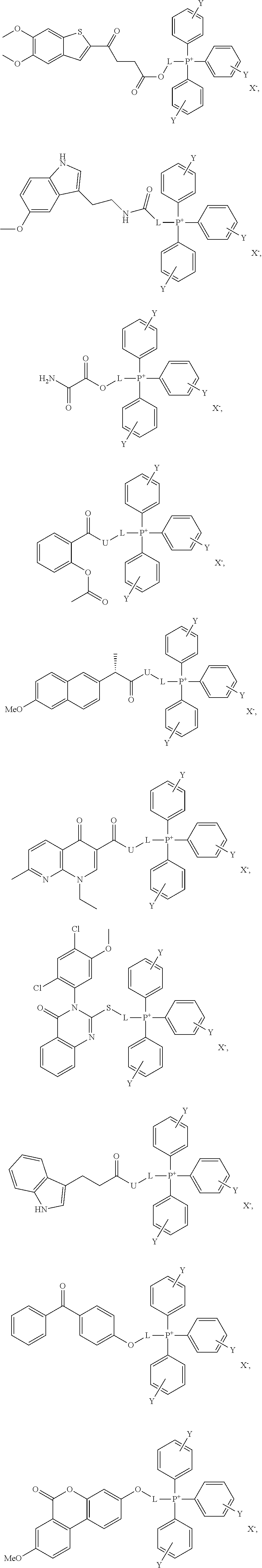

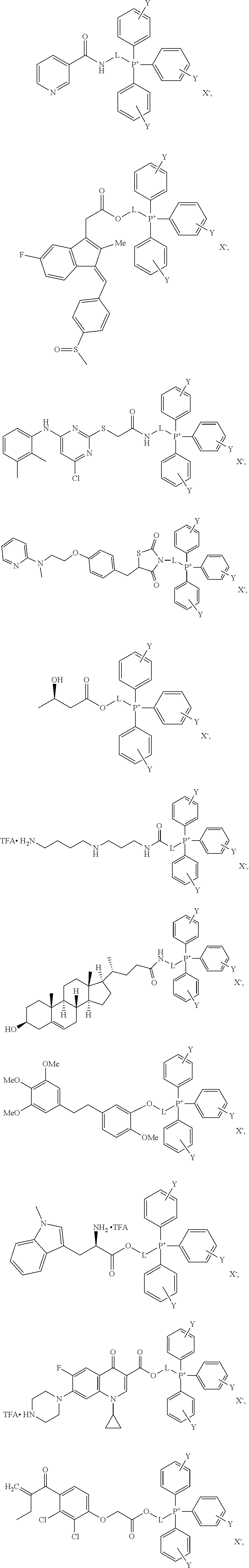

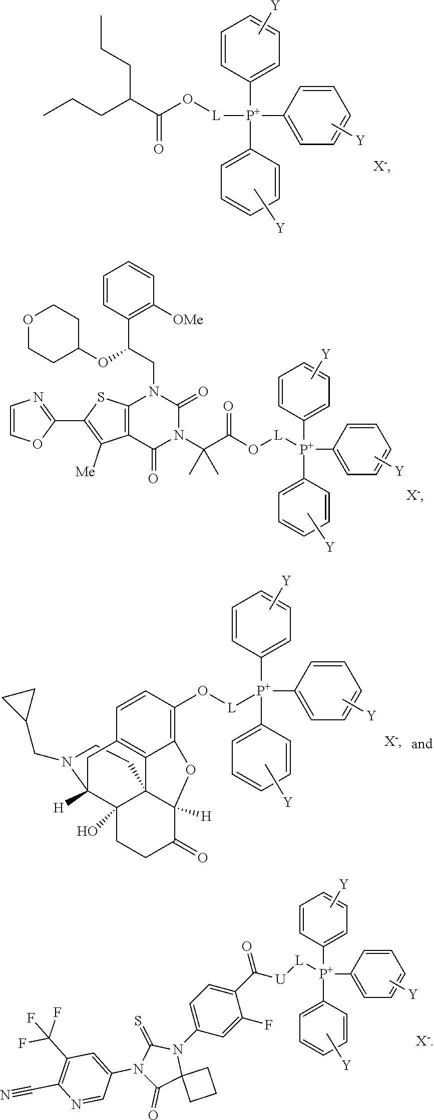

In some embodiments, the compound can be selected from:

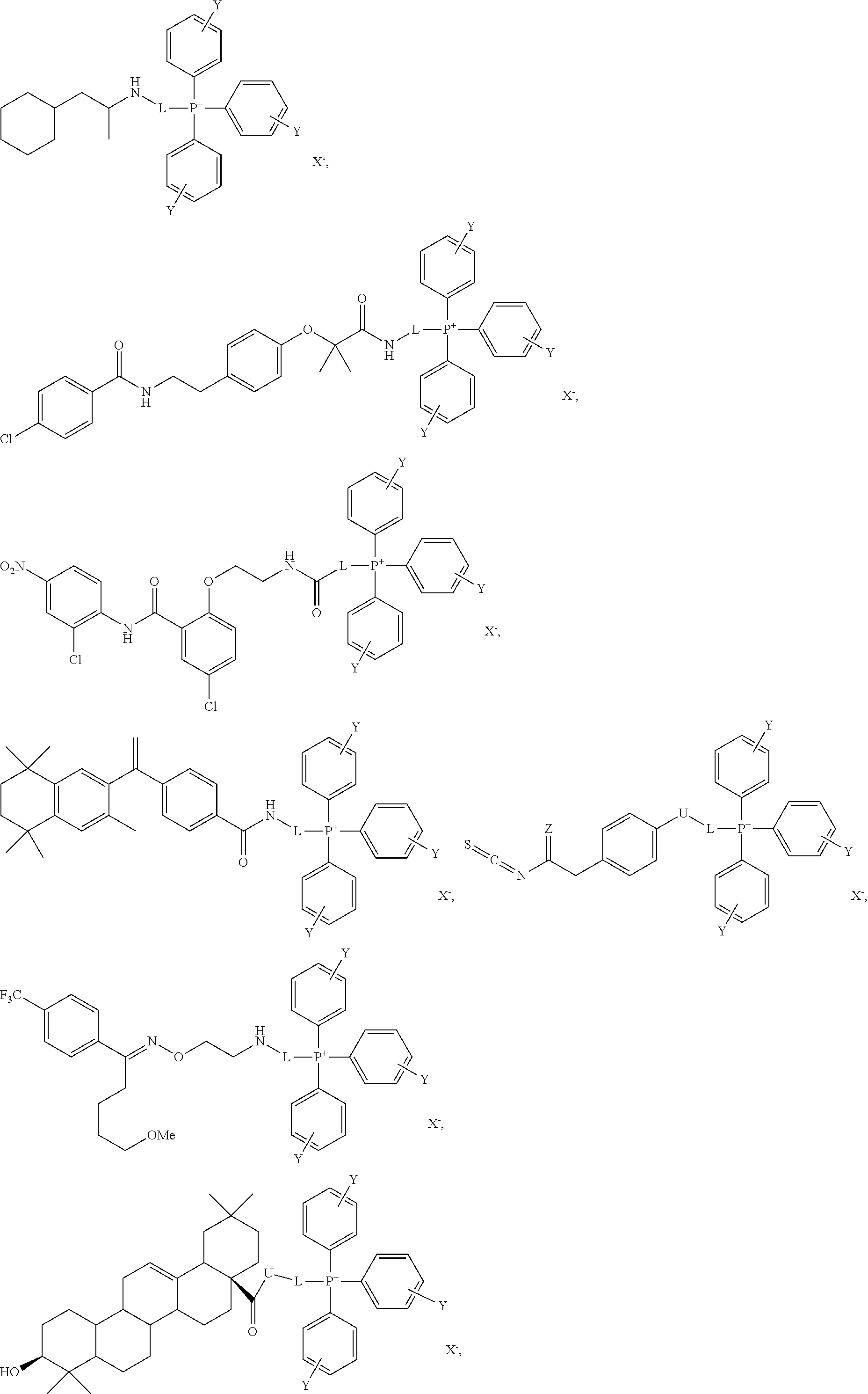

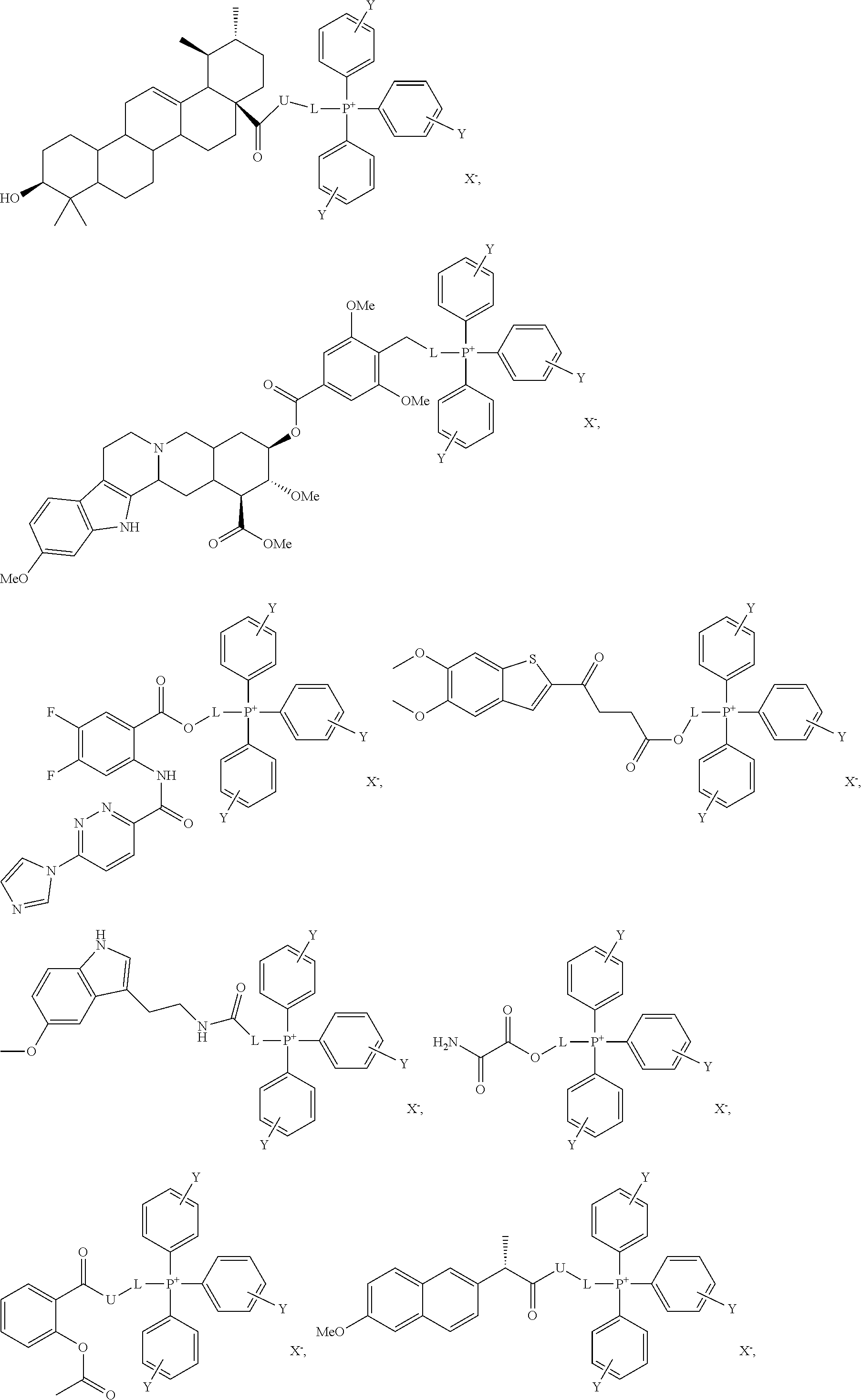

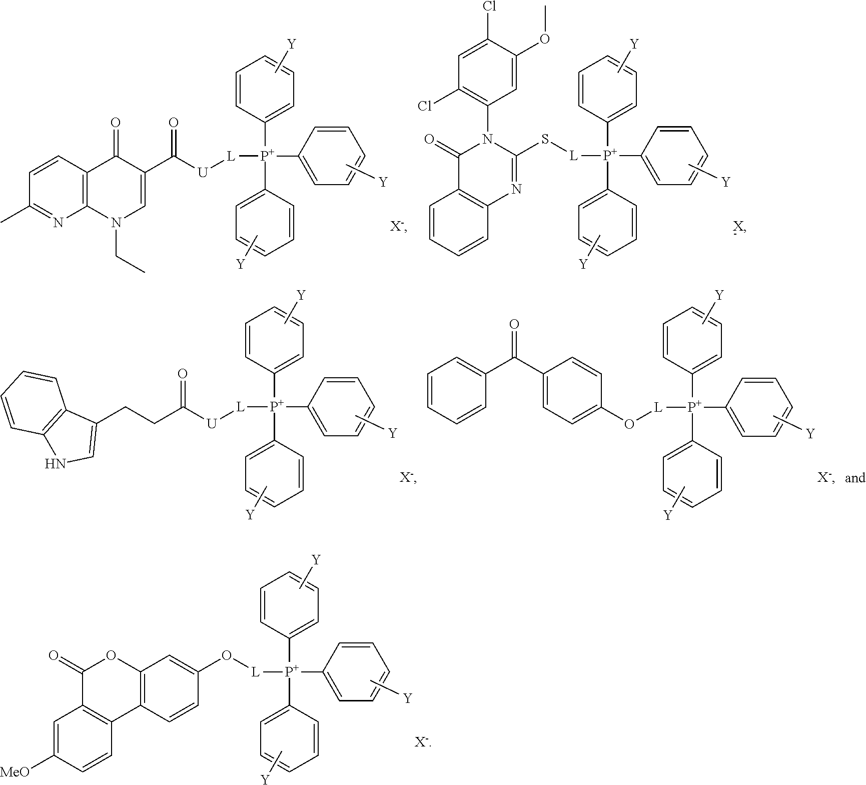

In some embodiments, the compound can be selected from:

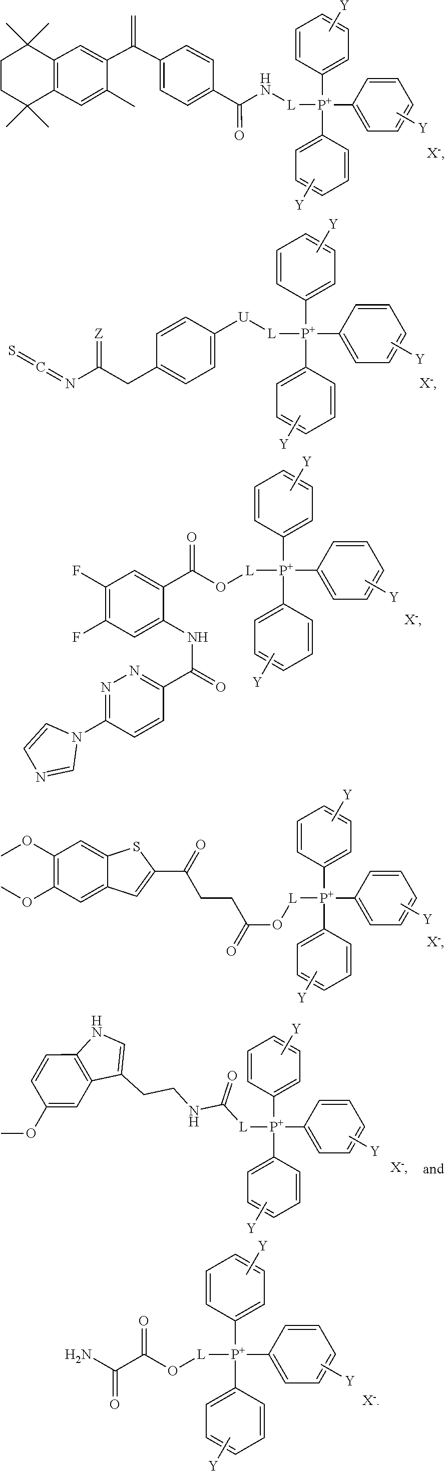

In some embodiments, the compound can be selected from:

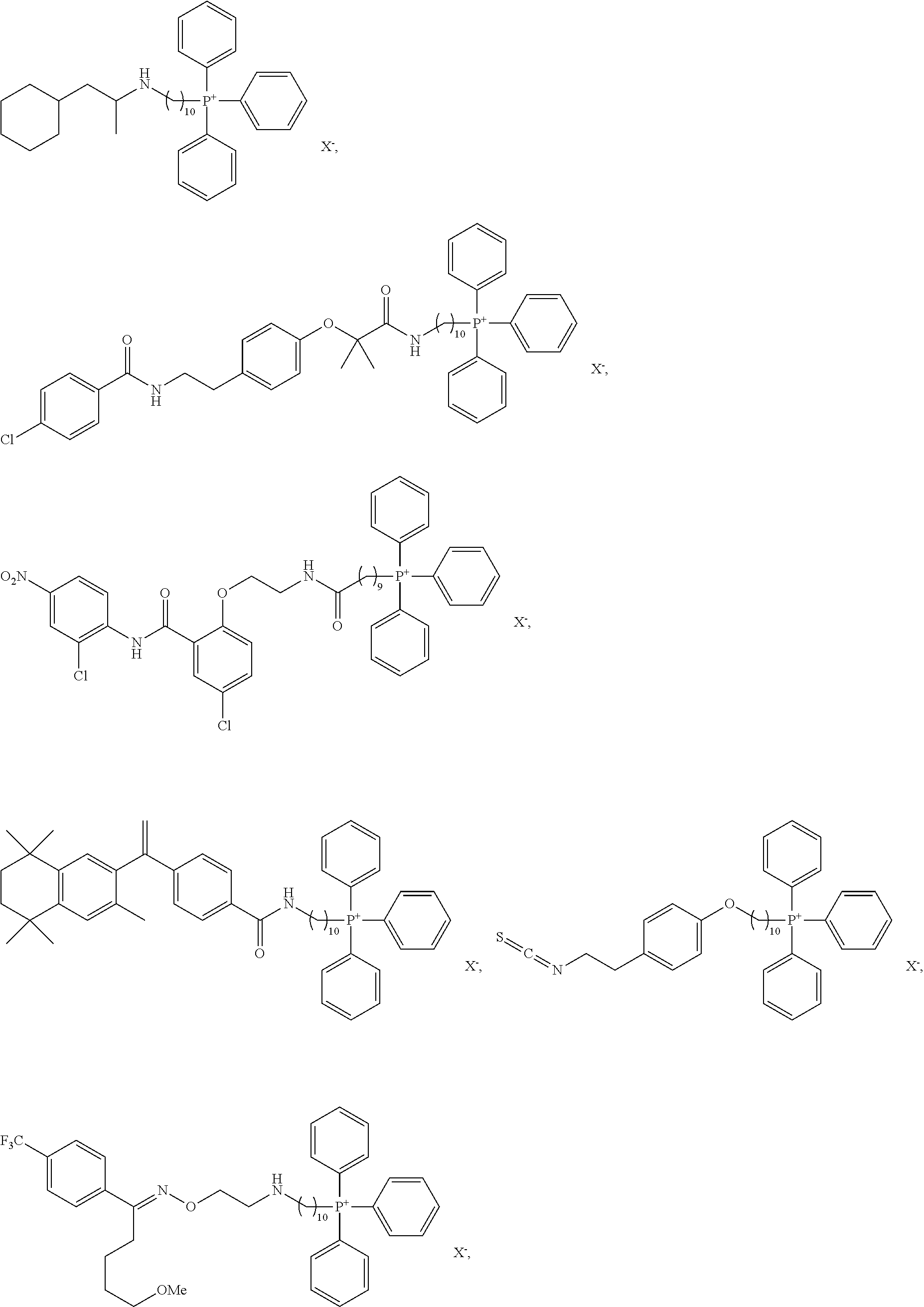

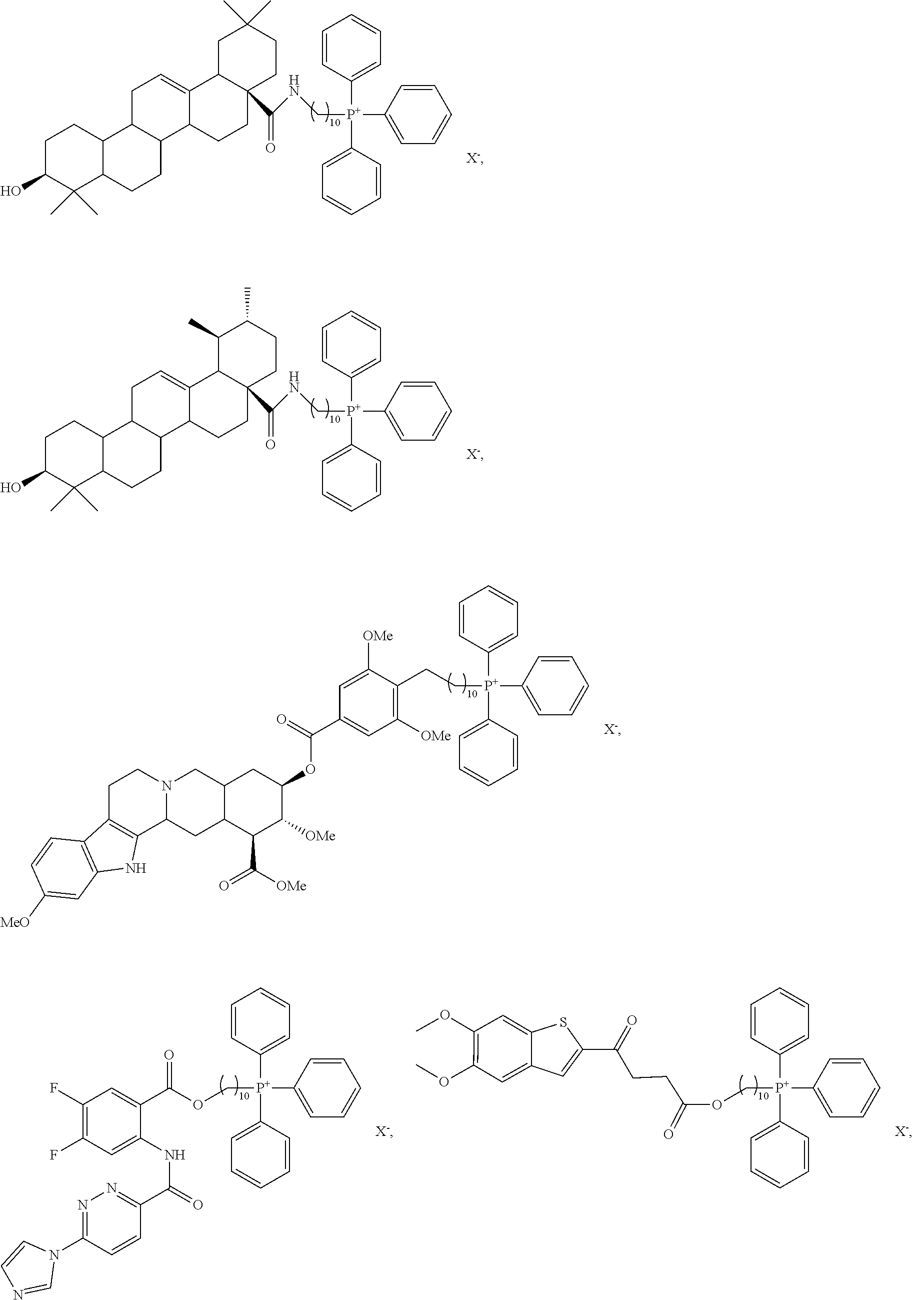

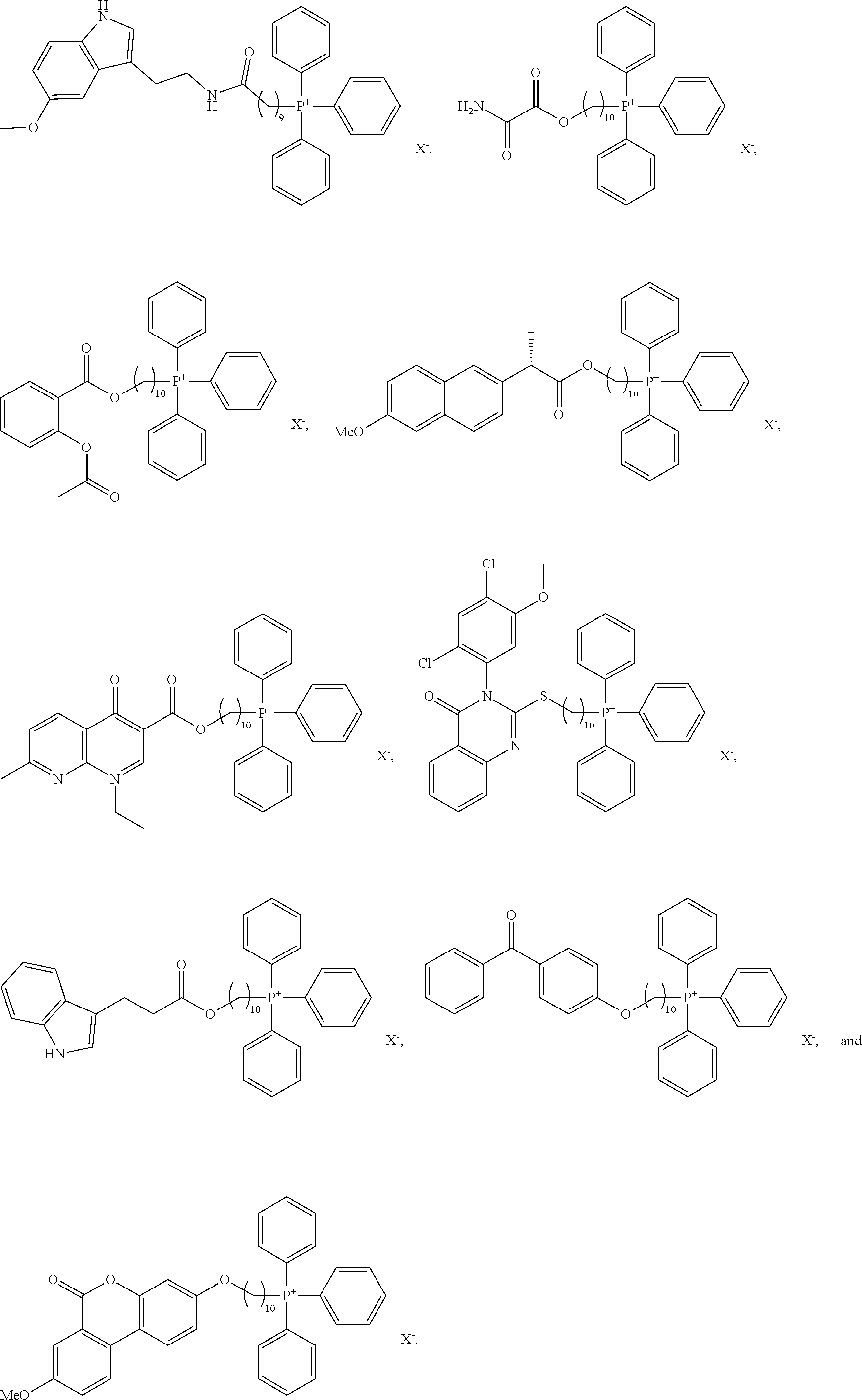

In some embodiments, the compound can be:

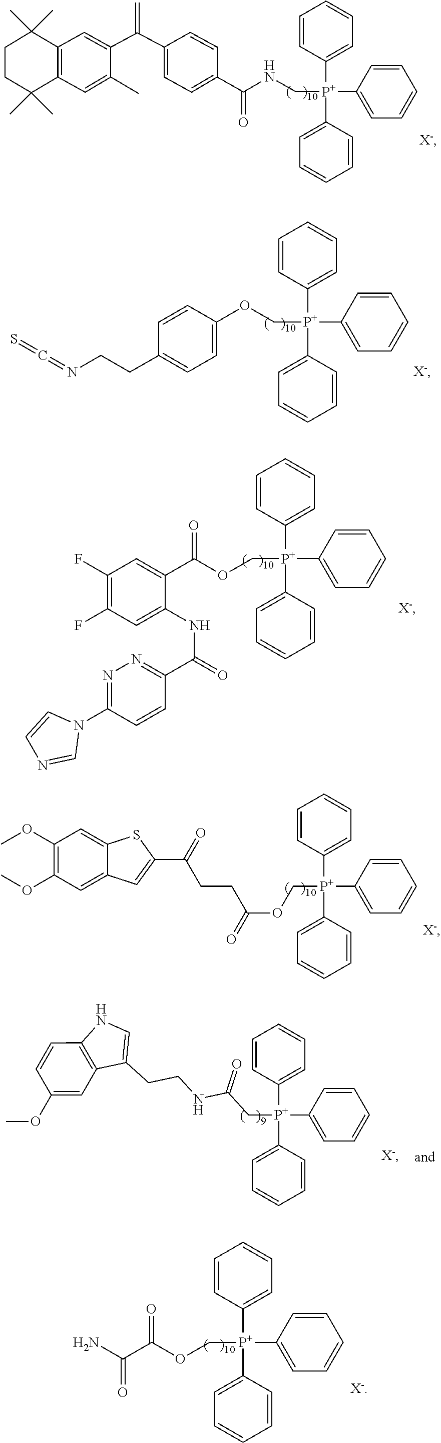

In some embodiments, the compound can be:

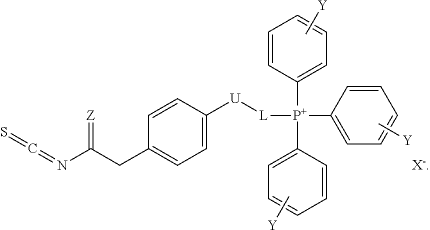

### Pharmaceutical Compositions

Described herein are pharmaceutical compositions including an effective amount of a compound described herein and an acceptable carrier.

The term “pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired.

General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHIPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The compounds can be incorporated microparticles, nanoparticles, or combinations thereof that provide controlled release of the compounds and/or additional active agents. For example, the compounds can be incorporated into polymeric microparticles, which provide controlled release of the drug(s). Release of the drug(s) is controlled by diffusion of the drug(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, may also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the compound can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of drug containing microparticles. Additionally, proteins, polysaccharides and combinations thereof, which are water-soluble, can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof.

Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug(s) in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above.

Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

The compounds described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication require polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, wafers, or extruded into a device, such as rods.

The release of the compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

In some embodiments, the pharmaceutical compositions can be administered locally. In some embodiments, the compounds are incorporated in a delivery system such as gels, nanoparticles, microparticles, or implants such as (e.g., rods, discs, wafers, orthopedic implants) for sustained release. In some embodiments, the compounds can be administered using a local delivery implantable system comprising the compounds incorporated within a gel, nanoparticles, microparticles, or an implant. In some embodiments, the pharmaceutical compositions comprise a delivery system such as gels, nanoparticles, microparticles, or implants such as (e.g., rods, discs, wafers, orthopedic implants) for sustained release of the active agent or a pharmaceutically acceptable salt or derivative thereof.

Methods of Use Described herein are methods for treating and/or preventing cancer, the methods including administering an effective amount of a compound described herein, or an effective amount of a composition described herein.

In some embodiments, the compounds, compositions and methods described herein are useful in treating or preventing a cancer. In some embodiments, the compositions herein are used to treat both local and metastatic tumors. In some cases, the cancer is a circulating cancer cell (circulating tumor cell). In some embodiments, the compounds, compositions and methods described herein are useful for treating or preventing metastasis or recurrence of a cancer. In some embodiments, the compounds, compositions and methods described herein are useful for the prevention of recurrence of excised solid tumors. In some embodiments, the compounds, compositions and methods described herein are useful for the prevention of metastasis of excised solid tumors.

In one aspect, the methods described herein are used to treat cancer, for example, melanoma, lung cancer (including lung adenocarcinoma, basal cell carcinoma, squamous cell carcinoma, large cell carcinoma, bronchioloalveolar carcinoma, bronchogenic carcinoma, non-small-cell carcinoma, small cell carcinoma, mesothelioma); breast cancer (including ductal carcinoma, lobular carcinoma, inflammatory breast cancer, clear cell carcinoma, mucinous carcinoma, serosal cavities breast carcinoma); colorectal cancer (colon cancer, rectal cancer, colorectal adenocarcinoma); anal cancer; pancreatic cancer (including pancreatic adenocarcinoma, islet cell carcinoma, neuroendocrine tumors); prostate cancer; prostate adenocarcinoma; ovarian carcinoma (ovarian epithelial carcinoma or surface epithelial-stromal tumor including serous tumor, endometrioid tumor and mucinous cystadenocarcinoma, sex-cord-stromal tumor); liver and bile duct carcinoma (including hepatocellular carcinoma, cholangiocarcinoma, hemangioma); esophageal carcinoma (including esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (including endometrial adenocarcinoma, ocular, uterine papillary serous carcinoma, uterine clear-cell carcinoma, uterine sarcomas, leiomyosarcomas, mixed mullerian tumors); glioma, glioblastoma, medulloblastoma, and other tumors of the brain; kidney cancers (including renal cell carcinoma, clear cell carcinoma, Wilm's tumor); cancer of the head and neck (including squamous cell carcinomas); cancer of the stomach (gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin, including melanoma, cervical, retinoblastoma, head and neck cancer, pancreatic, brain, thyroid, testicular, renal, bladder, soft tissue, adenal gland, urethra, cancers of the penis, myxosarcoma, chondrosarcoma, osteosarcoma, chordoma, malignant fibrous histiocytoma, lymphangiosarcoma, mesothelioma, squamous cell carcinoma; epidermoid carcinoma, malignant skin adnexal tumors, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma anaplastic; glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, pheochromocytoma, Islet cell carcinoma, malignant carcinoid, malignant paraganglioma, melanoma, Merkel cell neoplasm, cystosarcoma phylloide, salivary cancers, thymic carcinomas, and cancers of the vagina among others.

In some embodiments, the cancer is lung cancer. In some embodiments, the cancer is brain metastases from lung cancer.

### Methods of Administration

The compositions as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. The active agent may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc. Administration of the active components of their compositions can be a single administration, or at continuous and distinct intervals as can be readily determined by a person skilled in the art.

In certain embodiments, it may be desirable to provide continuous delivery of one or more compounds to a patient in need thereof. For intravenous or intraarterial routes, this can be accomplished using drip systems, such as by intravenous administration. For topical applications, repeated application can be done or a patch can be used to provide continuous administration of the compounds over an extended period of time.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

In some embodiments, the compound as used in the methods described herein may be administered in combination or alternation with one or more additional active agents. Representative examples of additional active agetns include antimicrobrial agetns (including antibiotics, antiviral agetns, and antifunfal agents), anti-inflamatory agetns (including steroids and non-steroidal anti-inflammatory agents), anti-coagulant agetns, and anti-cancer angents.

Exemplary cancer drugs or anti-cancer agents can include, but are not limited to, antimetabolite anti-cancer agents and antimitotic anti-cancer agents, and combinations thereof. Various antimetabolite and antimitotic anti-cancer agents, including single such agents or combinations of such agents, may be employed in the methods and compositions described herein.

Antimetabolic anti-cancer agents typically structurally resemble natural metabolites, which are involved in normal metabolic processes of cancer cells such as the synthesis of nucleic acids and proteins. The antimetabolites, however, differ enough from the natural metabolites such that they interfere with the metabolic processes of cancer cells. In the cell, antimetabolites are mistaken for the metabolites they resemble, and are processed by the cell in a manner analogous to the normal compounds. The presence of the “decoy” metabolites prevents the cells from carrying out vital functions and the cells are unable to grow and survive. For example, antimetabolites may exert cytotoxic activity by substituting these fraudulent nucleotides into cellular DNA, thereby disrupting cellular division, or by inhibition of critical cellular enzymes, which prevents replication of DNA.

In one aspect, therefore, the antimetabolite anti-cancer agent is a nucleotide or a nucleotide analog. In certain aspects, for example, the antimetabolite agent may comprise purine (e.g., guanine or adenosine) or analogs thereof, or pyrimidine (cytidine or thymidine) or analogs thereof, with or without an attached sugar moiety.

Suitable antimetabolite anti-cancer agents for use in the present disclosure may be generally classified according to the metabolic process they affect, and can include, but are not limited to, analogues and derivatives of folic acid, pyrimidines, purines, and cytidine. Thus, in one aspect, the antimetabolite agent(s) is selected from the group consisting of cytidine analogs, folic acid analogs, purine analogs, pyrimidine analogs, and combinations thereof.

In one particular aspect, for example, the antimetabolite agent is a cytidine analog. According to this aspect, for example, the cytidine analog may be selected from the group consisting of cytarabine (cytosine arabinodside), azacitidine (5-azacytidine), and salts, analogs, and derivatives thereof.

In another particular aspect, for example, the antimetabolite agent is a folic acid analog. Folic acid analogs or antifolates generally function by inhibiting dihydrofolate reductase (DHFR), an enzyme involved in the formation of nucleotides; when this enzyme is blocked, nucleotides are not formed, disrupting DNA replication and cell division. According to certain aspects, for example, the folic acid analog may be selected from the group consisting of denopterin, methotrexate (amethopterin), pemetrexed, pteropterin, raltitrexed, trimetrexate, and salts, analogs, and derivatives thereof.

In another particular aspect, for example, the antimetabolite agent is a purine analog. Purine-based antimetabolite agents function by inhibiting DNA synthesis, for example, by interfering with the production of purine containing nucleotides, adenine and guanine which halts DNA synthesis and thereby cell division. Purine analogs can also be incorporated into the DNA molecule itself during DNA synthesis, which can interfere with cell division. According to certain aspects, for example, the purine analog may be selected from the group consisting of acyclovir, allopurinol, 2-aminoadenosine, arabinosyl adenine (ara-A), azacitidine, azathiprine, 8-aza-adenosine, 8-fluoro-adenosine, 8-methoxy-adenosine, 8-oxo-adenosine, cladribine, deoxycoformycin, fludarabine, gancylovir, 8-aza-guanosine, 8-fluoro-guanosine, 8-methoxy-guanosine, 8-oxo-guanosine, guanosine diphosphate, guanosine diphosphate-beta-L-2-aminofucose, guanosine diphosphate-D-arabinose, guanosine diphosphate-2-fluorofucose, guanosine diphosphate fucose, mercaptopurine (6-MP), pentostatin, thiamiprine, thioguanine (6-TG), and salts, analogs, and derivatives thereof.

In yet another particular aspect, for example, the antimetabolite agent is a pyrimidine analog. Similar to the purine analogs discussed above, pyrimidine-based antimetabolite agents block the synthesis of pyrimidine-containing nucleotides (cytosine and thymine in DNA; cytosine and uracil in RNA). By acting as “decoys,” the pyrimidine-based compounds can prevent the production of nucleotides, and/or can be incorporated into a growing DNA chain and lead to its termination. According to certain aspects, for example, the pyrimidine analog may be selected from the group consisting of ancitabine, azacitidine, 6-azauridine, bromouracil (e.g., 5-bromouracil), capecitabine, carmofur, chlorouracil (e.g. 5-chlorouracil), cytarabine (cytosine arabinoside), cytosine, dideoxyuridine, 3′-azido-3′-deoxythymidine, 3′-dideoxycytidin-2′-ene, 3′-deoxy-3′-deoxythymidin-2′-ene, dihydrouracil, doxifluridine, enocitabine, floxuridine, 5-fluorocytosine, 2-fluorodeoxycytidine, 3-fluoro-3′-deoxythymidine, fluorouracil (e.g., 5-fluorouracil (also known as 5-FU), gemcitabine, 5-methylcytosine, 5-propynylcytosine, 5-propynylthymine, 5-propynyluracil, thymine, uracil, uridine, and salts, analogs, and derivatives thereof. In one aspect, the pyrimidine analog is other than 5-fluorouracil. In another aspect, the pyrimidine analog is gemcitabine or a salt thereof.

In certain aspects, the antimetabolite agent is selected from the group consisting of 5-fluorouracil, capecitabine, 6-mercaptopurine, methotrexate, gemcitabine, cytarabine, fludarabine, pemetrexed, and salts, analogs, derivatives, and combinations thereof. In other aspects, the antimetabolite agent is selected from the group consisting of capecitabine, 6-mercaptopurine, methotrexate, gemcitabine, cytarabine, fludarabine, pemetrexed, and salts, analogs, derivatives, and combinations thereof. In one particular aspect, the antimetabolite agent is other than 5-fluorouracil. In a particularly preferred aspect, the antimetabolite agent is gemcitabine or a salt or thereof (e.g., gemcitabine HCl (Gemzar®)).

Other antimetabolite anti-cancer agents may be selected from, but are not limited to, the group consisting of acanthifolic acid, aminothiadiazole, brequinar sodium, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, Wellcome EHNA, Merck & Co. EX-015, fazarabine, fludarabine phosphate, N-(2′-furanidyl)-5-fluorouracil, Daiichi Seiyaku FO-152, 5-FU-fibrinogen, isopropyl pyrrolizine, Lilly LY-188011; Lilly LY-264618, methobenzaprim, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, tiazofurin, Erbamont TIF, tyrosine kinase inhibitors, Taiho UFT and uricytin, among others.

In one aspect, the antimitotic anti-cancer agent is a microtubule inhibitor or a microtubule stabilizer. In general, microtubule stabilizers, such as taxanes and epothilones, bind to the interior surface of the beta-microtubule chain and enhance microtubule assembly by promoting the nucleation and elongation phases of the polymerization reaction and by reducing the critical tubulin subunit concentration required for microtubules to assemble. Unlike mictrotubule inhibitors, such as the vinca alkaloids, which prevent microtubule assembly, the microtubule stabilizers, such as taxanes, decrease the lag time and dramatically shift the dynamic equilibrium between tubulin dimers and microtubule polymers towards polymerization. In one aspect, therefore, the microtubule stabilizer is a taxane or an epothilone. In another aspect, the microtubule inhibitor is a vinca alkaloid.

In some embodiments, the anti-cancer agent may comprise a taxane or derivative or analog thereof. The taxane may be a naturally derived compound or a related form, or may be a chemically synthesized compound or a derivative thereof, with antineoplastic properties. The taxanes are a family of terpenes, including, but not limited to paclitaxel (Taxol®) and docetaxel (Taxotere®), which are derived primarily from the Pacific yew tree, Taxus brevifolia, and which have activity against certain tumors, particularly breast and ovarian tumors. In one aspect, the taxane is docetaxel or paclitaxel. Paclitaxel is a preferred taxane and is considered an antimitotic agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions.

Also included are a variety of known taxane derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; deoxygenated paclitaxel compounds such as those described in U.S. Pat. No. 5,440,056; and taxol derivatives described in U.S. Pat. No. 5,415,869. As noted above, it further includes prodrugs of paclitaxel including, but not limited to, those described in WO 98/58927; WO 98/13059; and U.S. Pat. No. 5,824,701. The taxane may also be a taxane conjugate such as, for example, paclitaxel-PEG, paclitaxel-dextran, paclitaxel-xylose, docetaxel-PEG, docetaxel-dextran, docetaxel-xylose, and the like. Other derivatives are mentioned in “Synthesis and Anticancer Activity of Taxol Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elsevier, Amsterdam 1986), among other references. Each of these references is hereby incorporated by reference herein in its entirety.

Various taxanes may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267) (each of which is hereby incorporated by reference herein in its entirety), or obtained from a variety of commercial sources, including for example, Sigma-Aldrich Co., St. Louis, Mo.

Alternatively, the antimitotic anti-cancer agent can be a microtubule inhibitor; in one preferred aspect, the microtubule inhibitor is a vinca alkaloid. In general, the vinca alkaloids are mitotic spindle poisons. The vinca alkaloid agents act during mitosis when chromosomes are split and begin to migrate along the tubules of the mitosis spindle towards one of its poles, prior to cell separation. Under the action of these spindle poisons, the spindle becomes disorganized by the dispersion of chromosomes during mitosis, affecting cellular reproduction. According to certain aspects, for example, the vinca alkaloid is selected from the group consisting of vinblastine, vincristine, vindesine, vinorelbine, and salts, analogs, and derivatives thereof.

The antimitotic anti-cancer agent can also be an epothilone. In general, members of the epothilone class of compounds stabilize microtubule function according to mechanisms similar to those of the taxanes. Epothilones can also cause cell cycle arrest at the G2-M transition phase, leading to cytotoxicity and eventually apoptosis. Suitable epithiolones include epothilone A, epothilone B, epothilone C, epothilone D, epothilone E, and epothilone F, and salts, analogs, and derivatives thereof. One particular epothilone analog is an epothilone B analog, ixabepilone (Ixempra™).

In certain aspects, the antimitotic anti-cancer agent is selected from the group consisting of taxanes, epothilones, vinca alkaloids, and salts and combinations thereof. Thus, for example, in one aspect the antimitotic agent is a taxane. More preferably in this aspect the antimitotic agent is paclitaxel or docetaxel, still more preferably paclitaxel. In another aspect, the antimitotic agent is an epothilone (e.g., an epothilone B analog). In another aspect, the antimitotic agent is a vinca alkaloid.

Examples of cancer drugs that may be used in the present disclosure include, but are not limited to: thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as sunitimib and imatinib.

Examples of additional cancer drugs include alkylating agents, antimetabolites, natural products, hormones and antagonists, and miscellaneous agents. Alternate names are indicated in parentheses. Examples of alkylating agents include nitrogen mustards such as mechlorethamine, cyclophosphainide, ifosfamide, melphalan sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, SFU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel, protein bound paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, histrelin, bleomycin, mitomycin (mitomycin C), idarubicin, epirubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, rnedroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Alternate names and trade-names of these and additional examples of cancer drugs, and their methods of use including dosing and administration regimens, will be known to a person versed in the art.

In some aspects, the anti-cancer agent may comprise a chemotherapeutic agent. Suitable chemotherapeutic agents include, but are not limited to, alkylating agents, antibiotic agents, antimetabolic agents, hormonal agents, plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof.

According to these aspects, the chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof. Each possibility represents a separate aspect of the invention.

Anti-neoplastic agent can be selected from the group consisting of Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Akynzeo (Netupitant and Palonosetron Hydrochloride), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Becenum (Carmustine), Beleodaq (Belinostat), Belinostat, Bendamustine Hydrochloride, BEP, Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPOX, Carboplatin, CARBOPLATIN-TAXOL, Carfilzomib, Carmubris (Carmustine), Carmustine, Carmustine Implant, Casodex (Bicalutamide), CeeNU (Lomustine), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cyramza (Ramucirumab), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), Imatinib Mesylate, Imbruvica (Ibrutinib), Imiquimod, Inlyta (Axitinib), Interferon Alfa-2b, Recombinant, Intron A (Recombinant Interferon Alfa-2b), Iodine 1131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Netupitant and Palonosetron Hydrochloride, Neupogen (Filgrastim), Nexavar (Sorafenib Tosylate), Nilotinib, Nivolumab, Nolvadex (Tamoxifen Citrate), Nplate (Romiplostim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Ontak (Denileukin Diftitox), Opdivo (Nivolumab), OPPA, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, R-EPOCH, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Thiotepa, Toposar (Etoposide), Topotecan Hydrochloride, Toremifene, Torisel (Temsirolimus), Tositumomab and Iodine I 131 Tositumomab, Totect (Dexrazoxane Hydrochloride), TPF, Trametinib, Trastuzumab, Treanda (Bendamustine Hydrochloride), Trisenox (Arsenic Trioxide), Tykerb (Lapatinib Ditosylate), Unituxin (Dinutuximab), Vandetanib, VAMP, Vectibix (Panitumumab), VeTP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate).

Growth factors useful as therapeutic agents include, but are not limited to, transforming growth factor-α (“TGF-α”), transforming growth factors (“TGF-β”), platelet-derived growth factors (“PDGF”), fibroblast growth factors (“FGF”), including FGF acidic isoforms 1 and 2, FGF basic form 2 and FGF 4, 8, 9 and 10, nerve growth factors (“NGF”) including NGF 2.5 s, NGF 7.0 s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, beta3, skeletal growth factor, bone matrix derived growth factors, and bone derived growth factors and mixtures thereof.

Immunoglobulins useful in the present disclosure include, but are not limited to, IgG, IgA, IgM, IgD, IgE, and mixtures thereof. Some preferred growth factors include VEGF (vascular endothelial growth factor), NGFs (nerve growth factors), PDGF-AA, PDGF-BB, PDGF-AB, FGFb, FGFa, and BGF.

Other molecules useful as anti-cancer agents include but are not limited to growth hormones, leptin, leukemia inhibitory factor (LIF), tumor necrosis factor alpha and beta, endostatin, thrombospondin, osteogenic protein-1, bone morphogenetic proteins 2 and 7, osteonectin, somatomedin-like peptide, osteocalcin.

Tumor antigens can be based on specific mutations (neoepitopes) and those expressed by cancer-germline genes (antigens common to tumors found in multiple patients, referred to herein as “traditional cancer antigens” or “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

Diagnostic agents include gases; metals; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

Reference will now be made in detail to specific aspects of the disclosed materials, compounds, compositions, articles, and methods, examples of which are illustrated in the accompanying Examples and Figures.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

### Example 1 Mitochondria-Targeted Agents for Disease Prevention and Treatment

Synthesis of mitochondria-targeted agents (MTAs). There is an urgent need to develop new and effective cancer preventive/therapeutic agents, such as mitochondria-targeted drugs, that can prevent/treat cancer and prolong survival. Mitochondria, the central site for cellular energy production, have important functions in cell survival and death. There is a significant role for mitochondrial metabolism in promoting cancer development and progression, making mitochondria a promising target for cancer prevention/treatment. Conjugating delocalized lipophilic cations, such as triphenylphosphonium cation (TPP+), to compounds of interest is an effective approach for mitochondrial targeting. The hyperpolarized tumor cell membrane and mitochondrial membrane potential allow for selective accumulation of TPP+ conjugates in tumor cell mitochondria versus those in normal cells. This could enhance direct killing of precancerous, dysplastic, and tumor cells while minimizing potential toxicities to normal cells. This invention is on the development of multiple mitochondria targeted anticancer drugs for potential use in cancer prevention and therapy.

Mitochondria in cell growth and death. Mitochondria efficiently produce ATP via cellular respiration that is essential to fulfill cellular bioenergetic needs. The mitochondrial electron transport chain (ETC) generates a transmembrane proton gradient that is used to generate ATP (Zhao R Z, et al., International journal of molecular medicine. 2019; 44(1):3-15 (“Zhao, et al., 2019”)). Some electrons may be prematurely shunted to O2, mainly by ETC complexes I and III, which results in the generation of superoxide (O^{•-}). Mitochondrial superoxide dismutase can dismutate O⁻ to hydrogen peroxide (H2O2) (Zhao, et al., 2019). Therefore, mitochondria are well recognized for their potential to generate reactive oxygen species (ROS). In addition, many mitochondrial TCA cycle metabolites can be used as building blocks to produce nucleotides, amino acids, lipids, heme, and others. Mitochondria also have important roles in redox signaling. Mitochondria can also initiate the intrinsic pathway that promotes apoptosis and necrosis-like non-apoptotic cell death. Once receiving proapoptotic stimuli, such as from Bax and Bak proteins, Ca²⁺ overload, or other signals, mitochondrial outer membrane permeabilization (MOMP) is induced. Once MOMP is initiated, cytochrome c is irreversibly released from the mitochondrial intermembrane space to the cytosol (Tait S W G, et al., Cold Spring Harb Perspect Biol. 2013; 5(9):a008706 (“Tait, et al., 2013”)), where it facilitates apoptosome formation, which acts as a platform to activate caspase-9 and the resulting cascades that promote apoptosis (Tait, et al., 2013). Once the mitochondrial permeability transition pore complex (mPTPC) is opened, large amounts of solutes enter the mitochondria matrix, resulting in osmotic swelling which can lead to rupture of the inner and outer membranes, accompanied by loss of mitochondrial membrane potential (ΔΨm), decreased ATP generation and excess ROS production (Tait, et al., 2013).

Mitochondrial metabolism is required for cancer development. Otto Warburg and colleagues observed that cancer cells ferment large amounts of glucose to lactate even in the presence of oxygen (Warburg O, The Journal of Cancer Research. 1925; 9(1):148). In the 1970s, Efraim Racker named this phenomenon ‘aerobic glycolysis’ or the ‘Warburg Effect’ (Racker E., et al., American Scientist. 1972; 60(1):56-63). The ‘Warburg Effect’ led to historical viewpoints that cancer cell mitochondria are dysfunctional and that these defects in cancer cell mitochondria render them inconsequential for cancer development. While glycolysis plays an important role in tumorigenesis, mitochondria provide ATP and building blocks (for pyrimidine, amino acid and heme biosynthesis) for cancer cells (DeBerardinis R J, et al., Fundamentals of cancer metabolism. Sci Adv. 2016; 2(5):e1600200-e), increasing evidence supports a pivotal role for mitochondrial function in cancer viability. For example, tumor cells lacking mitochondrial DNA (mtDNA) display significantly slower growth and lower tumorigenesis potential (Morais R, et al., Cancer research. 1994; 54(14):3889-96; Cavalli L R, et al., the molecular biology journal of the American Association for Cancer Research. 1997; 8(11):1189-98; and Magda D, et al., BMC Genomics. 2008; 9:521). Mutations that inactivate tumor suppressor genes and activate oncogenes also alter mitochondrial metabolism. For example, amplification of the Myc oncogene leads to activation of genes that are essential for mitochondrial biogenesis and function (Morrish F, et al., Cold Spring Harb Perspect Med. 2014; 4(5):a014225). Loss of the retinoblastoma tumor suppressor retinoblastoma (RB1) induces mitochondrial protein translation and increases oxidative phosphorylation (OXPHOS) (Zacksenhaus E, et al., Trends in cancer. 2017; 3(11):768-79). A consequence of increased OXPHOS is the accumulation of low levels of mitochondrial ROS which have been shown to contribute to neoplastic transformation, cell proliferation and survival (Sabharwal S S, et al., Nature reviews Cancer. 2014; 14(11):709-21). Some cancer subtypes (e.g. sarcoma, liver cancer, lung cancer and skin cancer) rely predominantly on OXPHOS for ATP production (Moreno-Sanchez R, et al., The FEBS journal. 2007; 274(6):1393-418). Fatty acid beta-oxidation (FAO), a series of multi-step catabolic reactions that shorten fatty acids, generates NADH and FADH2 that shuttle into mitochondria to support the ETC. FAO enzymes are dysregulated in cancer; elevated key FAO enzymes and/or high FAO activities are seen in multiple cancer types, including triple negative breast cancer, ovarian cancer, glioma, and mutant KRAS-driven lung cancer (Ma Y, et al., Cancer letters. 2018; 435:92-100 (“Ma, et al., 2018”)). FAO has been implicated in contributing to cancer cell survival and proliferation, metastasis, drug resistance (Ma, et al., 2018) and cancer stemness (Kuo C-Y, et al., Cancer Commun (Lond). 2018; 38(1):47). Although a majority of cancer cells have functional mitochondria, some cancers have defects that result from mutations in mtDNA or nuclear DNA that encode mitochondrial proteins (Hsu C-C, et al., Exp Biol Med (Maywood). 2016; 241(12):1281-95 (“Hsu C-C, et al., 2016”)). Interestingly, malfunctioning mitochondria can ultimately contribute to the formation and progression of cancer via ROS-induced nuclear gene instability, a process called retrograde signaling (Hsu C-C, et al., 2016). Collectively, these findings indicate that mitochondrial metabolism plays a critical role in tumorigenesis and cancer development.

Mitochondrial targeting using triphenylphosphonium cation (TPP+). Compared to normal cells, tumor cells have notable transformations in bioenergetics, biosynthesis, and modulated signal transduction which support non-stop growth (Porporato P E, et al., Cell Research. 2018; 28(3):265-80). For some time, scientists have been working on mitochondrial-targeted agents with high selectivity for cancer cells versus normal cells. The mitochondrial membrane potential in cancer cells (−220 mV) is more hyperpolarized than that in normal cells (−140 mV) (Forrest M D., bioRxiv. 2015:025197). This discrepancy can be exploited with compounds such as delocalized lipophilic cations (DLCs) that selectively accumulate in cancer cell mitochondria (Modica-Napolitano J S, et al., Advanced drug delivery reviews. 2001; 49(1-2):63-70). The mitochondrial membrane potential (negative inside) can drive a 100- to 1000-fold uptake of cations. Another advantage of lipophilic cations is that their lipophilicity promotes their ability to cross the plasma membrane and the mitochondrial outer and inner membranes. Triphenylphosphonium cation (TPP⁺) is the best characterized and most widely used lipophilic cation among the nonpeptide-based strategies for delivering conjugated compounds into mitochondria. TPP⁺ contains a positively charged phosphorus surrounded by three lipophilic phenyl groups. Due to greater uptake of lipophilic cations into cancer cells, phosphonium salts by themselves have some anti-proliferative activities by disrupting mitochondrial membrane integrity and inhibiting respiration in several cancer cell lines in vitro and in human ovarian cancer models in vivo (Dhanya D, et al., Anti-cancer agents in medicinal chemistry. 2017; 17(13):1796-804, Manetta A, et al., Gynecologic oncology. 1996; 60(2):203-12). Thus, TPP⁺ has been used to deliver various potential anticancer compounds including, but not limited to, antagonists of heat shock proteins, polyphenolic compounds, metabolic modulating agents, triterpernoids, and others (Bryant K G, et al., Oncotarget. 2017; 8(68):112184-98; Pan J, et al., iScience. 2018; 3:192-207; Cheng G, et al., Nature communications. 2019; 10(1):2205; and Tsepaeva O V, et al., Anti-cancer agents in medicinal chemistry. 2019). Summarized here are recent efforts and the success in the use of TPP⁺ for synthesizing a series of mitochondria-targeted compounds.

### Structures and NMR Characterizations of MTAs

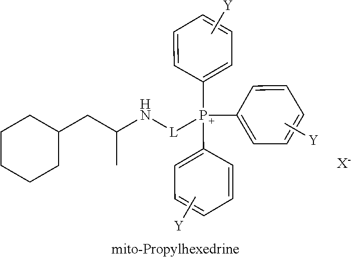

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR, ¹³C NMR and ³¹P NMR (L=(CH₂)₁₀, Y═H)

¹H NMR (600 MHz, CDCl₃) δ 8.94 (br, 1H), 7.83-7.78 (m, 9H), 7.74-7.70 (m, 6H), 3.74-3.68 (m, 2H), 3.36-3.29 (m, 1H), 2.94-2.86 (m, 2H), 2.08-2.02 (m, 2H), 1.85 (dt, J=11.6, 3.1 Hz, 1H), 1.76 (dt, J=11.2, 3.9 Hz, 1H), 1.72-1.56 (m, 9H), 1.48 (d, J=6.5 Hz, 3H), 1.40-1.27 (m, 9H), 1.27-1.17 (m, 3H), 1.17-1.07 (m, 2H), 1.01-0.94 (m, 1H), 0.94-0.85 (m, 1H).

¹³C NMR (150 MHz, CDCl₃) δ 135.1 (d, J=2.9 Hz), 133.7 (d, J=9.9 Hz), 130.5 (d, J=12.8 Hz), 118.4 (d, J=85.9 Hz), 53.0, 45.0, 39.3, 34.3 (d, J=18.9 Hz), 31.6, 29.7, 29.6, 27.9, 27.6, 27.5, 26.9, 26.4, 26.3, 26.0, 25.7, 25.1, 22.7 (d, J=49.5 Hz), 22.4 (d, J=4.6 Hz), 16.2.

³¹P NMR (243 MHz, CDCl₃) δ 24.3.

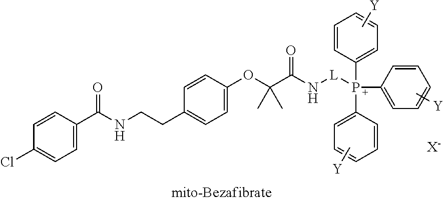

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR, ¹³C NMR and ³¹P NMR (L=(CH₂)₁₀, Y═H)

¹H NMR (600 MHz, CDCl₃) δ 7.79 (t, J=7.1 Hz, 3H), 7.72-7.66 (m, 7H), 7.66-7.61 (m, 5H), 7.33 (d, J=8.5 Hz, 1H), 7.14 (d, J=8.4 Hz, 1H), 6.83 (d, J=8.4 Hz, 1H), 6.70 (t, J=5.7 Hz, 1H), 6.58 (t, J=5.5 Hz, 1H), 3.63 (q, J=7.2 Hz, 2H), 3.25 (q, J=6.7 Hz, 2H), 3.19-3.08 (m, 3H), 2.87 (t, J=7.4 Hz, 2H), 1.83-1.79 (m, 1H), 1.62-1.55 (m, 2H), 1.55-1.49 (m, 2H), 1.50-1.41 (m, 8H), 1.31-1.12 (m, 10H).

¹³C NMR (151 MHz, CDCl₃) δ 174.7, 166.3, 152.9, 137.3, 135.3 (d, J=2.9 Hz), 133.3 (d, J=9.8 Hz), 130.6 (d, J=12.5 Hz), 130.5, 129.6, 128.7, 128.4, 121.0, 117.9 (d, J=85.6 Hz), 81.2, 41.5, 39.3, 38.6, 34.8, 30.2 (d, J=16.1 Hz), 29.2, 29.1, 29.0 (2C), 28.8, 26.7, 25.2 (2C), 22.5 (d, J=4.6 Hz), 22.1 (d, J=51.0 Hz).

³¹P NMR (243 MHz, CDCl₃) δ 24.3.

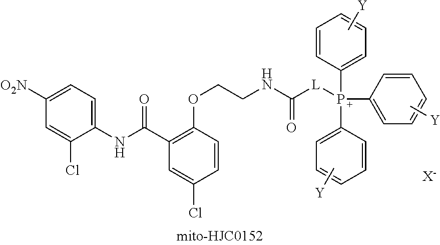

wherein L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR, ¹³C NMR and ³¹P NMR (L=(CH₂)₉, Y═H)

¹H NMR (600 MHz, CDCl₃) δ 10.66 (s, 1H), 8.77 (d, J=9.2 Hz, 1H), 8.22-8.15 (m, 2H), 8.10 (d, J=2.6 Hz, 1H), 8.07 (dd, J=9.6, 2.4 Hz, 1H), 7.79 (t, J=6.3 Hz, 3H), 7.76-7.71 (m, 6H), 7.71-7.65 (m, 6H), 7.38 (dd, J=8.9, 2.7 Hz, 1H), 7.32 (d, J=9.0 Hz, 1H), 4.47 (t, J=7.0 Hz, 2H), 3.71 (q, J=6.6 Hz, 2H), 3.52-3.41 (m, 2H), 2.24 (t, J=7.5 Hz, 2H), 2.12 (s, 1H), 1.65-1.45 (m, 6H), 1.32-1.08 (m, 9H).

¹³C NMR (151 MHz, CDCl₃) δ 174.6, 162.7, 155.2, 142.8, 141.4, 135.2 (d, J=2.9 Hz), 134.0, 133.5 (d, J=9.9 Hz), 131.9, 130.6 (d, J=12.6 Hz), 126.7, 124.6, 123.4, 123.2, 122.0, 121.2, 117.7 (d, J=85.7 Hz), 115.0, 67.8, 37.7, 35.9, 29.6, 29.5, 27.8, 27.7 (2C), 27.1, 24.8, 22.5 (d, J=50.1 Hz), 22.3 (d, J=4.4 Hz).

³¹P NMR (243 MHz, CDCl₃) δ 24.3.

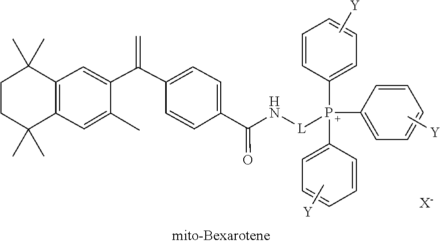

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR, ¹³C NMR (L=(CH₂)₁₀, Y═H)

¹H NMR (600 MHz, CDCl₃) δ 7.79 (dd, J=7.4, 6.3 Hz, 3H), 7.73-7.62 (m, 14H), 7.31 (d, J=8.3 Hz, 2H), 7.11 (s, 1H), 7.06 (s, 1H), 6.37 (t, J=5.6 Hz, 1H), 5.75 (s, 1H), 5.26 (s, 1H), 3.39 (q, J=6.8 Hz, 2H), 3.18-3.01 (m, 2H), 1.92 (s, 3H), 1.38-1.00 (m, 22H).

¹³C NMR (151 MHz, CDCl₃) δ 167.2, 149.2, 144.2, 143.9, 142.2, 138.2, 135.2 (d, J=2.5 Hz), 133.3 (d, J=9.8 Hz), 133.3, 130.6 (d, J=12.5 Hz), 130.5, 128.0 (2C), 126.9, 126.7, 118.0 (d, J=86.4 Hz), 116.1, 40.0, 35.2 (2C), 34.0, 33.9, 31.9 (4C), 29.3, 28.8 (2C), 28.6, 26.7, 22.4 (d, J=4.4 Hz), 22.1 (d, J=51.4 Hz), 19.9.

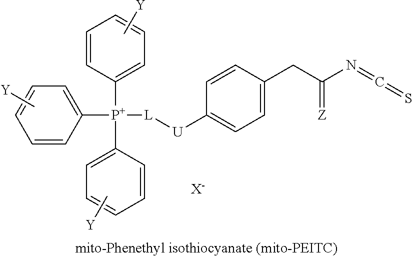

wherein
U is oxygen or sulfur;
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position;
X is any halogen, TFA, triflate, hexafluorophosphate or acetic acid; and
Z is —H₂ or —CH₂CH₂—.

¹H NMR, ¹³C NMR and ³¹P NMR (L=(CH₂)₁₀, U═O, Z═H₂, Y═H)

¹H NMR (600 MHz, CDCl₃) δ 7.81-7.74 (m, 9H), 7.72-7.66 (m, 6H), 7.10 (d, J=8.5 Hz, 2H), 6.84 (d, J=8.5 Hz, 2H), 3.91 (t, J=6.5 Hz, 2H), 3.67 (t, J=6.9 Hz, 2H), 3.62-3.44 (m, 2H), 2.91 (t, J=6.9 Hz, 2H), 1.79-1.67 (m, 2H), 1.64-1.51 (m, 4H), 1.44-1.35 (m, 2H), 1.32-1.13 (m, 8H).

¹³C NMR (151 MHz, CDCl₃) δ 158.3, 135.0 (d, J=2.8 Hz), 133.4 (d, J=9.8 Hz), 130.4 (d, J=12.6 Hz), 130.4, 129.7, 128.7, 118.4 (d, J=86.0 Hz), 114.8, 68.0, 46.6, 35.7, 30.3 (d, J=16.0 Hz), 29.3, 29.2, 29.1, 29.0, 28.9, 25.9, 22.6, 22.5 (d, J=4.5 Hz), 21.9 (d, J=50.2 Hz).

³¹P NMR (243 MHz, CDCl₃) δ 24.2.

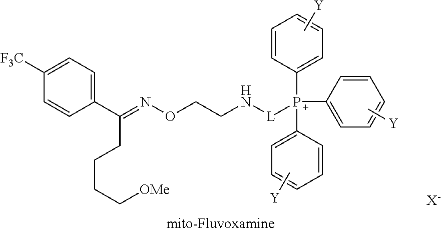

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR, ¹³C NMR and ³¹P NMR (L=(CH₂)₁₀, Y═H)

¹H NMR (600 MHz, CDCl₃) δ 7.81-7.71 (m, 9H), 7.70-7.60 (m, 8H), 7.57-7.48 (m, 2H), 4.28 (s, 1H), 3.66-3.53 (m, 2H), 3.23 (s, 3H), 2.91 (s, 1H), 2.73 (d, J=6.6 Hz, 2H), 2.61 (d, J=6.6 Hz, 1H), 2.47-2.15 (m, 3H), 1.70-1.37 (m, 10H), 1.26-1.04 (m, 10H).

¹³C NMR (151 MHz, CDCl₃) δ 157.2, 139.0, 134.9, 133.5 (d, J=10.0 Hz), 130.4 (d, J=12.5 Hz), 130.4, 126.4, 125.1 (q, J=3.8 Hz), 123.9 (q, J=273.3 Hz), 118.1 (d, J=86.1 Hz), 73.2, 72.0, 58.4, 49.6, 48.6, 30.2 (d, J=15.5 Hz), 29.6, 29.3, 29.2, 29.1, 28.9 (2C), 27.0, 25.9, 23.0, 22.8, 22.4.

³¹P NMR (243 MHz, CDCl₃) δ 24.4.

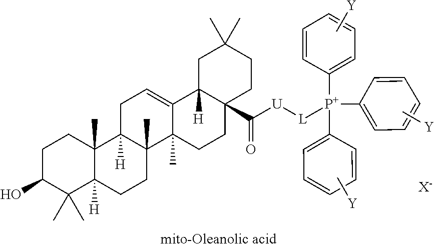

wherein
U is oxygen or —NH;
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR, ¹³C NMR and ³¹P NMR (L=(CH₂)₁₀, U═NH, Y═H)

¹H NMR (600 MHz, CDCl₃) δ 7.80 (t, J=7.3 Hz, 1H), 7.73-7.67 (m, 6H), 7.69-7.62 (m, 3H), 5.93 (t, J=5.2 Hz, 1H), 5.37 (s, 1H), 3.30 (dq, J=12.6, 6.6 Hz, 1H), 3.20 (dd, J=11.3, 4.1 Hz, 1H), 3.17-3.09 (m, 2H), 2.99-2.90 (m, 1H), 2.49 (d, J=9.6 Hz, 1H), 1.93-1.87 (m, 1H), 1.74 (t, J=13.2 Hz, 1H), 1.67 (d, J=14.0 Hz, 1H), 1.64-1.48 (m, 13H), 1.47-1.37 (m, 3H), 1.37-1.29 (m, 3H), 1.27-1.16 (m, 16H), 1.14 (s, 3H), 0.97 (s, 3H), 0.91-0.86 (m, 9H), 0.75 (d, J=10.2 Hz, 2H).

¹³C NMR (151 MHz, CDCl₃) δ 178.0, 144.9, 135.2 (d, J=2.9 Hz), 133.3 (d, J=9.8 Hz), 130.6 (d, J=12.6 Hz), 122.7, 117.9 (d, J=86.1 Hz), 78.9, 55.1, 47.5, 46.7, 46.2, 42.2, 42.0, 39.4, 39.3, 38.7, 38.4, 36.9, 34.1, 32.9, 32.4 (d, J=22.0 Hz), 30.7, 30.2 (d, J=16.0 Hz), 29.2, 29.0, 28.8, 28.1, 27.2 (d, J=22.8 Hz), 27.0, 25.7, 23.7, 23.5, 23.4, 22.4 (d, J=4.4 Hz), 22.1 (d, J=51.5 Hz), 18.3, 16.9, 15.5, 15.3.

³¹P NMR (243 MHz, CDCl₃) δ 24.1.

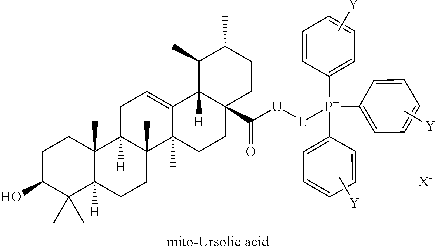

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR, ¹³C NMR and ³¹P NMR (L=(CH₂)₁₀, U═NH, Y═H)

¹H NMR (600 MHz, CDCl₃) δ 7.80 (t, J=7.3 Hz, 3H), 7.73-7.68 (m, 6H), 7.68-7.60 (m, 6H), 5.91 (t, J=5.2 Hz, 1H), 5.31 (s, 1H), 3.30-3.23 (m, 1H), 3.21 (dd, J=11.4, 4.5 Hz, 1H), 3.18-3.11 (m, 2H), 3.02-2.92 (m, 1H), 2.01-1.90 (m, 3H), 1.89-1.81 (m, 2H), 1.67-1.49 (m, 12H), 1.49-1.37 (m, 6H), 1.31-1.17 (m, 17H), 1.08 (s, 1H), 0.98 (s, 3H), 0.93 (s, 3H), 0.90 (s, 3H), 0.86 (d, J=6.4 Hz, 3H), 0.76 (s, 6H).

¹³C NMR (151 MHz, CDCl₃) δ 178.0, 139.9, 135.3 (d, J=2.9 Hz), 133.3 (d, J=9.9 Hz), 130.6 (d, J=12.5 Hz), 125.6, 118.0 (d, J=86.0 Hz), 79.0, 55.1, 53.8, 47.6, 47.5, 42.5, 39.8, 39.5, 39.4, 39.0, 38.7, 38.6, 37.2, 36.9, 32.8, 31.9, 31.0, 30.2 (d, J=15.6 Hz), 29.2, 29.1, 29.0, 28.8, 28.1, 27.8, 27.1, 27.0, 24.8, 23.4, 23.2, 22.5 (d, J=4.5 Hz), 22.1 (d, J=51.3 Hz), 21.2, 18.3, 17.2, 16.9, 15.6, 15.5.

³¹P NMR (243 MHz, CDCl₃) δ 23.4.

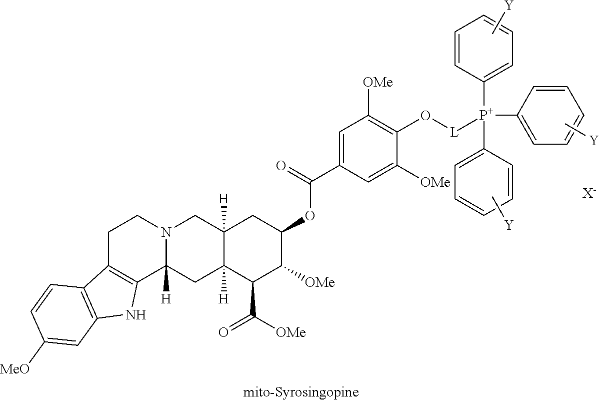

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR, ¹³C NMR and ³¹P NMR (L=(CH₂)₁₀, Y═H)

¹H NMR (600 MHz, DMSO) δ 11.04 (br, 1H), 7.89 (t, J=7.3 Hz, 3H), 7.86-7.79 (m, 6H), 7.79-7.72 (m, 6H), 7.33 (s, 3H), 6.87 (s, 1H), 6.68 (d, J=8.4 Hz, 1H), 5.76 (s, 1H), 4.95-4.87 (m, 1H), 3.95-3.87 (m, 3H), 3.87-3.79 (m, 9H), 3.76 (s, 3H), 3.66-3.57 (m, 2H), 3.43-3.19 (m, 6H), 3.03-2.72 (m, 3H), 2.48-2.29 (m, 2H), 2.29-2.10 (m, 2H), 2.00-1.84 (m, 2H), 1.65-1.58 (m, 2H), 1.58-1.42 (m, 4H), 1.42-1.33 (m, 2H), 1.31-1.15 (m, 9H).

¹³C NMR (151 MHz, DMSO) δ 171.7, 165.2, 156.4, 153.5, 141.7, 137.8, 135.3 (d, J=2.6 Hz), 134.0 (d, J=10.1 Hz), 130.7 (d, J=12.4 Hz), 130.7, 125.0, 119.1, 119.0 (d, J=85.5 Hz), 107.1, 105.7, 95.3, 77.7, 77.4, 73.0, 60.7, 56.6, 55.7, 55.4, 55.1, 52.6, 50.8, 48.2, 30.3, 30.2, 30.0, 29.4, 29.3, 29.1 (2C), 28.6, 25.7, 23.0, 22.2, 22.2, 20.9, 20.5, 15.9.

³¹P NMR (243 MHz, CDCl₃) δ 24.2.

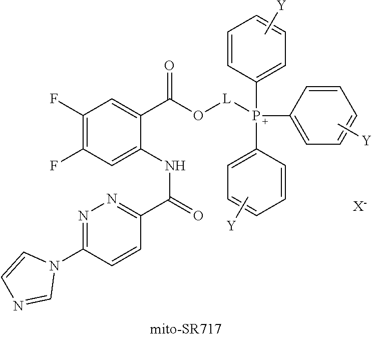

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR and ¹³C NMR (L=(CH₂)₁₀, Y═H)

¹H NMR (600 MHz, CDCl₃) δ 13.27 (s, 1H), 8.93 (dd, J=13.0, 7.4 Hz, 1H), 8.69 (s, 1H), 8.56 (d, J=9.1 Hz, 1H), 8.03 (d, J=9.0 Hz, 1H), 7.96 (dd, J=10.8, 8.7 Hz, 1H), 7.90-7.84 (m, 6H), 7.81 (td, J=7.4, 1.7 Hz, 3H), 7.72 (td, J=7.8, 3.3 Hz, 6H), 7.32 (s, 1H), 4.44 (t, J=6.7 Hz, 2H), 3.82 (td, J=10.7, 5.4 Hz, 2H), 1.82 (p, J=6.9 Hz, 2H), 1.61 (d, J=4.1 Hz, 4H), 1.39 (q, J=7.7 Hz, 2H), 1.34-1.16 (m, 8H).

¹³C NMR (151 MHz, CDCl₃) δ 166.05, 160.72, 154.42, 153.29, 151.85, 146.55, 144.91, 137.65, 137.58, 134.95, 134.93, 133.78, 133.71, 131.99, 130.49, 130.41, 129.23, 119.84, 119.71, 118.81, 118.24, 118.10, 116.38, 113.48, 110.25, 110.10, 66.48, 30.47, 29.33, 29.23, 29.15, 29.13, 28.47, 25.95, 22.97, 22.72, 22.64.

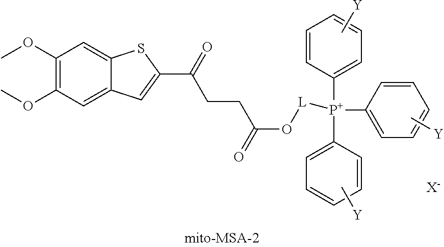

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR and ¹³C NMR (L=(CH₂)₁₀, Y═H)

¹H NMR (600 MHz, CDCl₃) δ 7.91 (s, 1H), 7.86-7.81 (m, 6H), 7.81-7.76 (m, 3H), 7.70 (td, J=7.7, 3.2 Hz, 6H), 7.27 (s, 1H), 7.25 (s, 1H), 4.06 (t, J=6.7 Hz, 2H), 3.97 (s, 3H), 3.94 (s, 3H), 3.82-3.74 (m, 2H), 3.31 (t, J=6.7 Hz, 2H), 2.76 (t, J=6.7 Hz, 2H), 1.67-1.46 (m, 6H), 1.31-1.11 (m, 10H).

¹³C NMR (151 MHz, CDCl₃) δ 192.02, 172.71, 151.07, 148.91, 141.13, 136.77, 134.96, 133.69, 133.62, 132.69, 130.52, 130.43, 129.14, 118.78, 118.21, 106.08, 64.85, 56.22, 56.12, 33.67, 32.55, 30.41, 30.31, 29.23, 29.07, 29.02, 28.50, 28.46, 25.76, 22.69, 22.66, 22.63, 22.37.

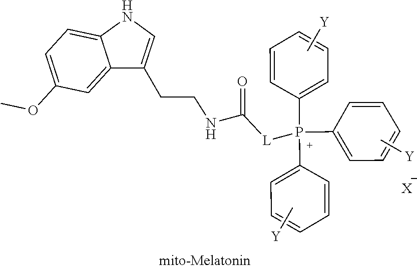

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR and ¹³C NMR (L=(CH₂)₉, Y═H)

¹H NMR (600 MHz, CDCl₃) δ 9.53 (s, 1H), 7.90-7.77 (m, 9H), 7.69 (td, J=7.8, 3.4 Hz, 6H), 7.40 (d, J=8.7 Hz, 1H), 7.07 (d, J=2.4 Hz, 1H), 7.03 (d, J=2.4 Hz, 1H), 6.79 (dd, J=8.7, 2.5 Hz, 1H), 6.15 (t, J=5.7 Hz, 1H), 3.85 (s, 3H), 3.80-3.70 (m, 2H), 3.58 (q, J=6.3 Hz, 2H), 2.95 (t, J=6.5 Hz, 2H), 2.16 (t, J=7.3 Hz, 2H), 1.66-1.60 (m, 4H), 1.54 (t, J=7.2 Hz, 2H), 1.34-1.26 (m, 2H), 1.24-1.17 (m, 6H).

¹³C NMR (151 MHz, CDCl₃) δ 173.36, 153.66, 135.00, 134.98, 133.68, 133.61, 131.96, 130.50, 130.45, 129.50, 129.41, 127.56, 123.48, 118.70, 118.13, 112.68, 111.76, 56.02, 39.19, 36.61, 30.16, 30.05, 28.59, 28.57, 28.49, 28.39, 25.49, 25.34, 22

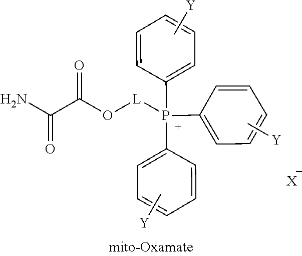

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles;
Each Y is independently selected from —H, —OMe, —OCF₃, CF₃, or Cl, and can be located at any ortho, meta and para position; and
X is any halogen, TFA, triflate, hexafluorophosphate or acetic acid.

¹H NMR and ¹³C NMR (L=(CH₂)₁₀, Y═H)

¹H NMR (600 MHz, Acetone) δ 8.07-8.03 (m, 6H), 7.94 (td, J=7.5, 1.7 Hz, 3H), 7.81 (dt, J=7.7, 3.8 Hz, 6H), 7.71 (s, 1H), 7.31 (s, 1H), 4.21 (t, J=6.6 Hz, 2H), 3.95-3.88 (m, 2H), 1.75-1.62 (m, 6H), 1.37-1.25 (m, 10H).

¹³C NMR (151 MHz, Acetone) δ 160.93, 158.48, 134.91, 134.89, 134.01, 133.94, 130.31, 130.22, 119.33, 118.76, 65.85, 32.79, 30.10, 29.99, 28.48, 28.13, 25.50, 22.24, 22.22, 21.74, 21.41.

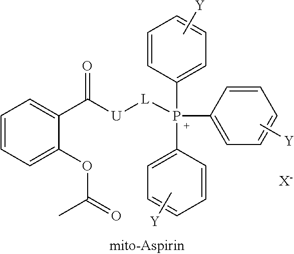

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
U is O, or NH.
X is any halogen, TFA, triflate, hexafluorophosphate or acetic acid.

¹H NMR, ¹³C NMR and ³¹P NMR (L=(CH₂)₁₀, U═O, Y═H)

¹H NMR (600 MHz, CDCl₃) δ 7.95 (dd, J=8.2, 1.0 Hz, 1H), 7.81-7.72 (m, 9H), 7.69-7.63 (m, 6H), 7.50 (td, J=8.0, 1.5 Hz, 1H), 7.26 (td, J=8.4, 1.0 Hz, 1H), 7.04 (dd, J=8.0, 0.8 Hz, 1H), 4.19 (t, J=7.1 Hz, 2H), 3.70-3.62 (m, 2H), 2.29 (s, 3H), 1.65 (quint, J=7.2 Hz, 2H), 1.61-1.52 (m, 4H), 1.35-1.27 (m, 2H), 1.26-1.11 (m, 8H).

¹³C NMR (150 MHz, CDCl₃) δ 169.5, 164.4, 150.5, 134.9 (d, J=3.2 Hz), 133.6, 133.5 (d, J=10.0 Hz), 131.6, 130.4 (d, J=12.6 Hz), 125.9, 123.6, 123.4, 118.1 (d, J=85.9 Hz), 65.1, 30.2 (d, J=15.5 Hz), 29.2, 29.0, 28.9, 28.4, 25.7, 22.8, 22.5, 22.4, 20.9.

³¹P NMR (243 MHz, CDCl₃) δ 24.5.

Results for the cell proliferation assay are shown in FIG. 25.

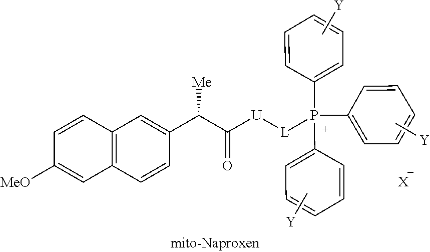

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
U is O, or NH.
X is any halogen, TFA, triflate, hexafluorophosphate or acetic acid.

¹H NMR, ¹³C NMR and ³¹P NMR (L=(CH₂)₁₀, U═O, Y═H)

¹H NMR (600 MHz, CDCl₃) δ 7.8-7.73 (m, 9H), 7.69-7.63 (m, 8H), 7.36 (d, J=7.8 Hz, 1H), 7.08-7.04 (m, 2H), 3.99 (t, J=6.6 Hz, 2H), 3.85 (s, 3H), 3.80 (q, J=7.4 Hz, 1H), 3.70-3.60 (m, 2H), 1.61-1.44 (m, 9H), 1.20-1.03 (m, 10H).

¹³C NMR (151 MHz, CDCl₃) δ 174.6, 157.4, 135.7, 134.9 (d, J=3.0 Hz), 133.5 (d, J=10.0 Hz), 133.5, 130.4 (d, J=12.7 Hz), 129.1, 128.8, 126.9, 126.2, 118.7, 118.1 (d, J=85.8 Hz), 105.5, 64.7, 55.2, 45.4, 30.2 (d, J=15.6 Hz), 29.1, 29.0, 28.9 (d, J=3.9 Hz), 28.3, 25.5, 22.8, 22.5, 22.4, 18.3.

³¹P NMR (243 MHz, CDCl₃) δ 24.4.

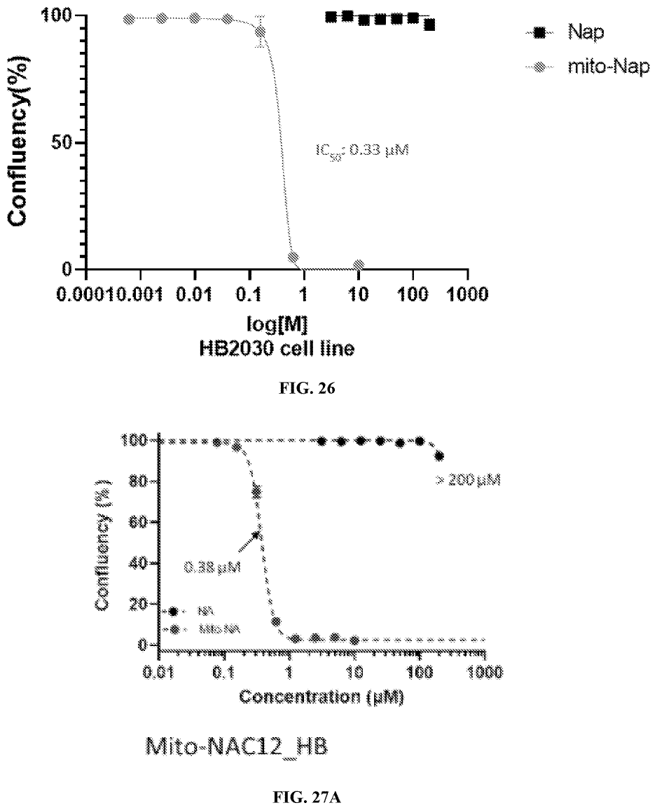

Results for the cell proliferation assay are shown in FIG. 26.

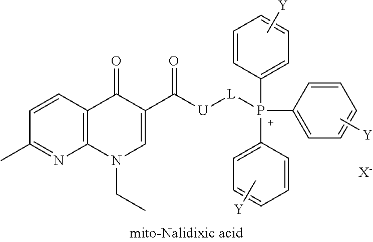

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
U is O, or NH.
X is any halogen, TFA, triflate, hexafluorophosphate or acetic acid.

¹H NMR, ¹³C NMR and ³¹P NMR (L=(CH₂)₁₀, U═O, Y═H)

¹H NMR (600 MHz, CDCl₃) δ 8.61 (d, J=7.9 Hz, 1H), 8.59 (s, 1H), 7.88-7.82 (m, 6H), 7.80-7.76 (m, 3H), 7.72-7.67 (m, 6H), 7.23 (d, J=8.0 Hz, 1H), 4.48 (q, J=7.2 Hz, 2H), 4.28 (t, J=7.2 Hz, 2H), 3.85-3.75 (m, 2H), 2.65 (s, 3H), 1.77-1.69 (m, 4H), 1.64-1.58 (m, 2H), 1.48 (t, J=7.2 Hz, 3H), 1.41-1.34 (m, 2H), 1.29-1.16 (m, 8H).

¹³C NMR (151 MHz, CDCl₃) δ 174.7, 165.5, 162.6, 148.6 (2C), 136.8, 134.9 (d, J=2.9 Hz), 133.7 (d, J=10.0 Hz), 130.4 (d, J=12.6 Hz), 121.5, 121.1, 118.5 (d, J=85.7 Hz), 112.0, 64.9, 46.6, 30.3 (d, J=15.6 Hz), 29.2, 29.1, 29.0, 28.7, 25.8, 25.1, 22.9, 22.6, 22.5, 15.2.

³¹P NMR (243 MHz, CDCl₃) δ 24.5.

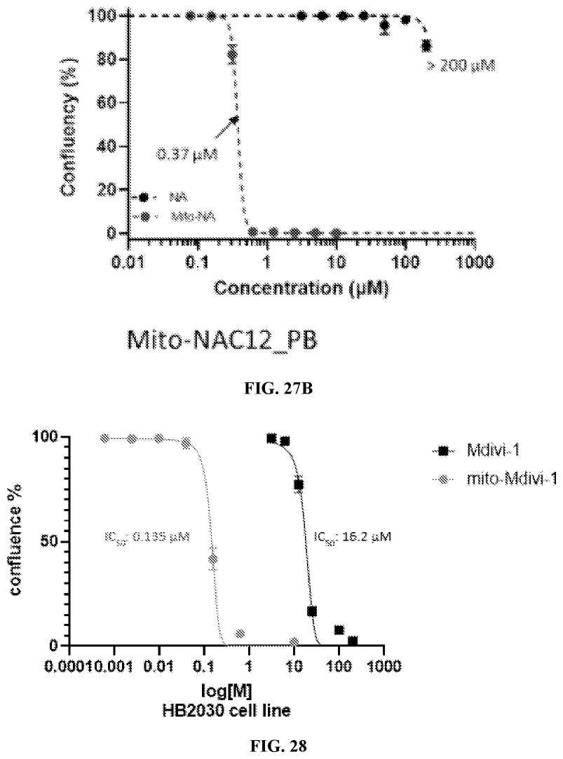

Results for the cell proliferation assay are shown in FIG. 27A-27B.

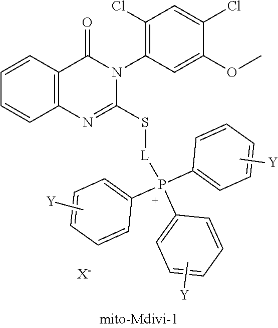

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR and ³¹P NMR (L=(CH₂)₁₀, Y═H)

¹H NMR (600 MHz, CDCl₃) δ 8.18 (dd, J=7.9, 1.0 Hz, 1H), 7.83-7.78 (m, 6H), 7.78-7.74 (m, 4H), 7.73-7.70 (m, 1H), 7.69-7.65 (m, 6H), 7.58 (d, J=8.1 Hz, 1H), 7.36 (t, J=7.6 Hz, 1H), 6.88 (s, 1H), 3.87 (s, 3H), 3.74-3.68 (m, 2H), 3.22-3.15 (m, 1H), 3.12-3.06 (m, 1H), 1.66-1.61 (m, 2H), 1.61-1.54 (m, 4H), 1.37-1.29 (m, 2H), 1.26-1.15 (m, 8H).

¹³C NMR (151 MHz, CDCl₃) δ 160.9, 156.4, 154.6, 147.8, 134.9 (d, J=2.9 Hz), 134.9, 133.6 (d, J=10.0 Hz), 132.7, 131.0, 130.4 (d, J=12.9 Hz), 127.1, 126.3, 125.8, 125.1, 124.9, 119.4, 118.3 (d, J=86.0 Hz), 113.9, 55.6, 45.1, 32.2, 30.3 (d, J=15.5 Hz), 29.2, 29.0, 28.9, 28.6, 28.4, 22.7 (d, J=40.1 Hz), 25.6 (d, J=4.9 Hz).

³¹P NMR (243 MHz, CDCl₃) δ 24.5.

Results for the cell proliferation assay are shown in FIG. 28.

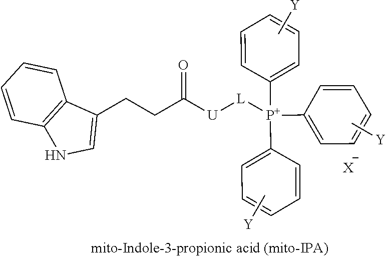

wherein
U is oxygen or sulfur.
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —Ome, —OCF₃, CF₃, or Cl, and can be located at any ortho, meta and para position.
U is O, or NH.
X is any halogen, TFA, triflate, hexafluorophosphate or acetic acid.

¹H NMR, ¹³C NMR and ³¹P NMR (L=(CH₂)₁₀, U═O, Y═H)

¹H NMR (600 MHz, CDCl₃) δ 9.23 (br, 1H), 7.80-7.69 (m, 9H), 7.68-7.61 (m, 6H), 7.54-7.50 (m, 1H), 7.39-7.36 (m, 1H), 7.04-6.99 (m, 2H), 4.02 (t, J=6.7 Hz, 2H), 3.61-3.52 (m, 2H), 3.04 (t, J=7.9 Hz, 2H), 2.66 (t, J=7.8 Hz, 2H), 1.60-1.48 (m, 6H), 1.25-1.11 (m, 10H).

¹³C NMR (151 MHz, CDCl₃) δ 173.5, 136.4, 135.0 (d, J=2.8 Hz), 133.5 (d, J=10.0 Hz), 130.4 (d, J=12.3 Hz), 127.1, 121.9, 121.3, 118.6, 118.2 (d, J=85.9 Hz), 118.2, 113.8, 111.7, 64.4, 35.1, 30.3 (d, J=15.9 Hz), 29.0, 28.9, 28.8, 28.3, 25.6, 22.8, 22.5 (d, J=4.5 Hz), 22.4, 20.8.

³¹P NMR (243 MHz, CDCl₃) δ 24.3.

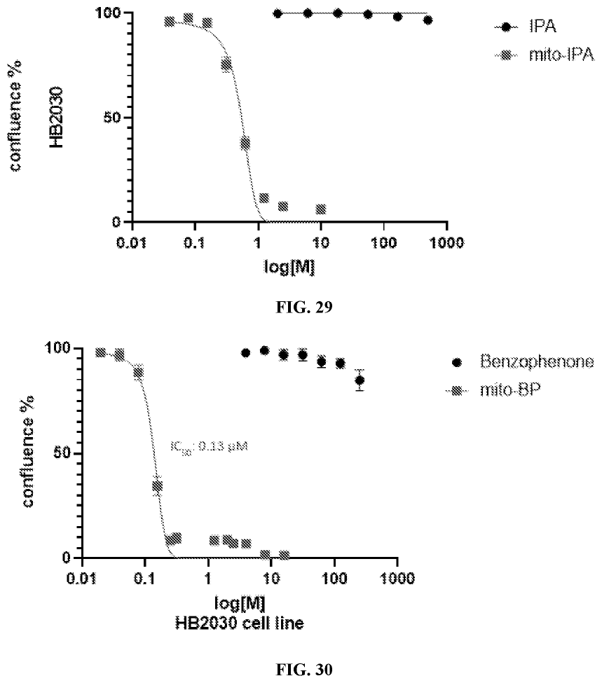

Results for the cell proliferation assay are shown in FIG. 29.

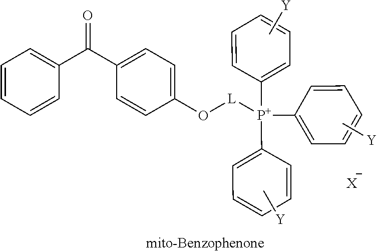

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR and ³¹P NMR (L=(CH₂)₁₀, Y═H)

¹H NMR (600 MHz, CDCl₃) δ 7.81-7.72 (m, 11H), 7.71-7.63 (m, 8H), 7.51 (tt, J=7.6, 1.5 Hz, 1H), 7.44-7.39 (m, 2H), 6.89 (d, J 8.4 Hz, 2H), 3.97 (t, J=7.0 Hz, 2H), 3.72-3.64 (m, 2H), 1.73 (quint, J=6.7 Hz, 2H), 1.62-1.53 (m, 4H), 1.41-1.33 (m, 2H), 1.29-1.14 (m, 8H).

¹³C NMR (151 MHz, CDCl₃) δ 195.4, 162.7, 138.1, 134.9 (d, J=2.8 Hz), 133.4 (d, J=9.9 Hz), 132.3, 131.7, 130.3 (d, J=12.6 Hz), 129.6, 129.5, 128.0, 118.1 (d, J=86.4 Hz), 113.9, 68.1, 30.2 (d, J=15.7 Hz), 29.1, 29.0, 28.9, 28.8, 25.7, 22.8, 22.5, 22.4.

³¹P NMR (243 MHz, CDCl₃) δ 24.5.

Results for the cell proliferation assay are shown in FIG. 30.

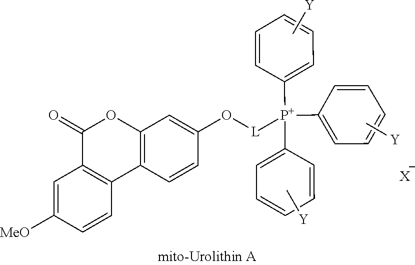

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR and ³¹P NMR (L=(CH₂)₁₀, Y═H)

¹H NMR (600 MHz, CDCl₃) δ 7.92 (d, J=9.1 Hz, 1H), 7.86-7.82 (m, 6H), 7.79-7.76 (m, 4H), 7.74 (d, J=2.8 Hz, 1H), 7.71-7.67 (m, 6H), 7.36 (dd, J=8.8, 2.9 Hz, 1H), 6.88 (dd, J=8.6, 2.4 Hz, 1H), 6.81 (d, J=2.4 Hz, 1H), 3.98 (t, J=6.4 Hz, 2H), 3.91 (s, 3H), 3.81-3.76 (m, 2H), 1.80-1.74 (m, 2H), 1.64-1.58 (m, 2H), 1.44-1.38 (m, 2H), 1.28-1.19 (m, 10H).

¹³C NMR (151 MHz, CDCl₃) δ 161.6, 160.2, 159.1, 151.6, 134.9 (d, J=3.0 Hz), 133.7 (d, J=10.0 Hz), 130.4 (d, J=12.9 Hz), 128.7, 124.4, 123.1, 122.8, 120.9, 118.4 (d, J=85.6 Hz), 112.7, 111.1, 111.0, 102.1, 68.4, 55.7, 45.2, 30.4 (d, J=15.5 Hz), 29.3, 29.2, 29.1, 28.9, 25.9, 22.7 (d, J=49.8 Hz), 22.6 (d, J=4.3 Hz) ³¹P NMR (243 MHz, CDCl₃) δ 24.4.

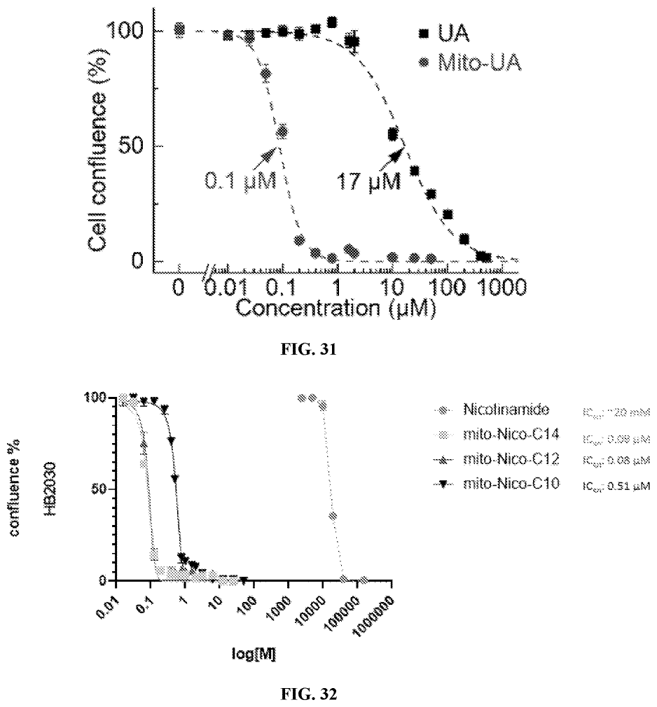

Results for the cell proliferation assay are shown in FIG. 31.

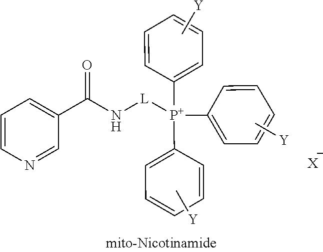

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, and ³¹P NMR (L=(CH₂)₁₀, Y═H)

¹H NMR (600 MHz, CDCl₃) δ 9.15 (dd, J=2.3, 0.8 Hz, 1H), 8.60 (dd, J=4.9, 1.6 Hz, 1H), 8.50 (dt, J=8.0, 2.0 Hz, 1H), 7.96 (br, 1H), 7.84-7.75 (m, 9H), 7.73-7.65 (m, 6H), 7.32 (dd, J=8.0, 4.8 Hz, 1H), 3.69 (m, 2H), 3.43 (q, J=6.0 Hz, 2H), 1.67 (m, 2H), 1.62-1.57 (m, 4H), 1.37-1.15 (m, 10H).

¹³C NMR (151 MHz, CDCl₃) δ 165.5 (2C), 165.5, 151.1, 148.8, 135.1 (d, J=3.2 Hz), 133.6 (d, J=10.0 Hz), 130.5 (d, J=12.4 Hz), 123.3, 118.3 (d, J=85.7 Hz), 39.9, 30.0 (d, J=16.0 Hz), 28.9, 28.5, 28.4, 28.3, 26.5, 22.9, 22.5 (2C).

³¹P NMR (243 MHz, CDCl₃) δ 24.3.

Results for the cell proliferation assay are shown in FIG. 32.

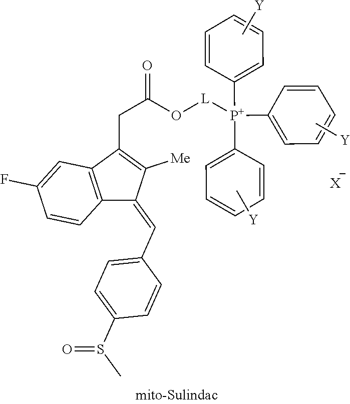

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, and ³¹P NMR (L=(CH₂)₁₀, Y═H)

¹H NMR (600 MHz, CDCl₃) δ 7.89-7.81 (m, 6H), 7.80-7.72 (m, 3H), 7.72-7.58 (m, 6H), 7.30-7.18 (m, 5H), 7.10-7.03 (m, 1H), 6.83-6.76 (m, 2H), 3.88-3.80 (m, 2H), 2.97-2.86 (m, 2H), 2.52 (s, 3H), 2.37-2.30 (m, 1H), 2.25-2.17 (m, 1H), 1.69 (s, 3H), 1.65-1.55 (m, 4H), 1.41-1.31 (m, 2H), 1.27-1.09 (m, 10H).

¹³C NMR (151 MHz, CDCl₃) δ 173.44, 164.21, 140.09, 138.41, 134.92, 134.90, 133.79, 133.77, 133.72, 133.44, 132.83, 130.48, 130.46, 130.39, 130.25, 128.74, 126.46, 126.33, 125.79, 118.84, 118.27, 116.34, 116.18, 95.38, 70.84, 43.91, 41.36, 29.25, 29.15, 29.06, 26.95, 22.95, 22.78, 22.75, 22.62, 18.29, 15.55.

165.5 (2C), 165.5, 151.1, 148.8, 135.1 (d, J=3.2 Hz), 133.6 (d, J=10.0 Hz), 130.5 (d, J=12.4 Hz), 123.3, 118.3 (d, J=85.7 Hz), 39.9, 30.0 (d, J=16.0 Hz), 28.9, 28.5, 28.4, 28.3, 26.5, 22.9, 22.5 (2C).

³¹P NMR (243 MHz, CDCl₃) δ 24.5.

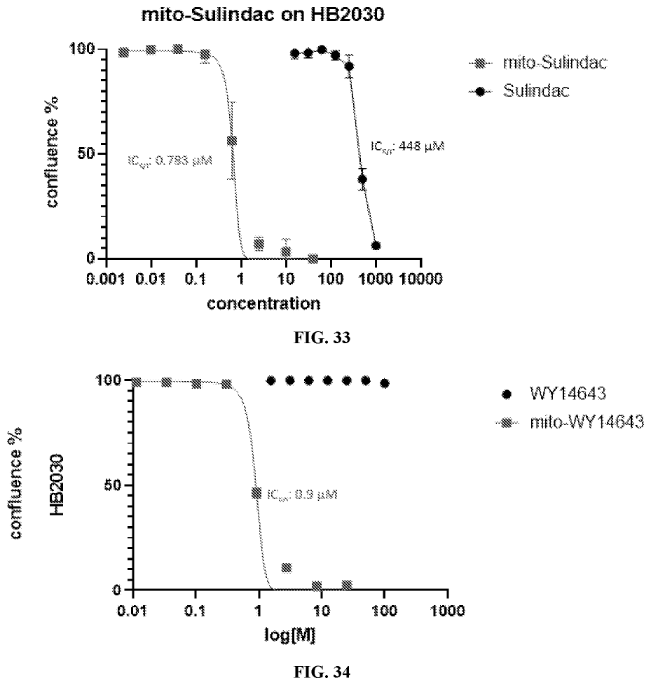

Results for the cell proliferation assay are shown in FIG. 33.

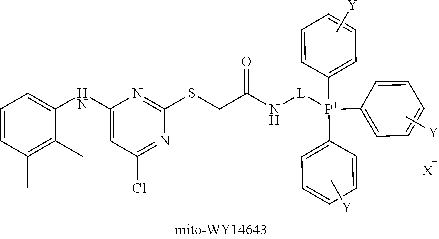

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, and ³¹P NMR (L=(CH₂)₁₀, Y═H)

¹H NMR (600 MHz, CDCl₃) δ 9.15 (dd, J=2.3, 0.8 Hz, 1H), 8.60 (dd, J=4.9, 1.6 Hz, 1H), 8.50 (dt, J=8.0, 2.0 Hz, 1H), 7.96 (br, 1H), 7.84-7.75 (m, 9H), 7.73-7.65 (m, 6H), 7.32 (dd, J=8.0, 4.8 Hz, 1H), 3.69 (m, 2H), 3.43 (q, J=6.0 Hz, 2H), 1.67 (m, 2H), 1.62-1.57 (m, 4H), 1.37-1.15 (m, 10H).

¹³C NMR (151 MHz, CDCl₃) δ 165.5 (2C), 165.5, 151.1, 148.8, 135.1 (d, J=3.2 Hz), 133.6 (d, J=10.0 Hz), 130.5 (d, J=12.4 Hz), 123.3, 118.3 (d, J=85.7 Hz), 39.9, 30.0 (d, J=16.0 Hz), 28.9, 28.5, 28.4, 28.3, 26.5, 22.9, 22.5 (2C).

³¹P NMR (243 MHz, CDCl₃) δ 24.3.

Results for the cell proliferation assay are shown in FIG. 34.

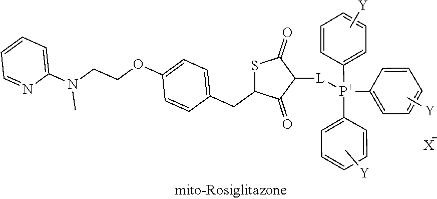

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, and ³¹P NMR (L=(CH₂)₁₀, Y═H)

¹H NMR (600 MHz, CDCl₃) δ 8.07 (d, J=6.7 Hz, 1H), 7.82-7.71 (m, 10H), 7.70-7.62 (m, 6H), 7.39 (ddd, J=8.9, 7.0, 2.1 Hz, 1H), 7.05 (d, J=8.2 Hz, 2H), 6.77 (d, J=8.8 Hz, 2H), 6.51-6.44 (m, 2H), 4.38-4.31 (m, 1H), 4.13-4.04 (m, 2H), 3.91 (t, J=5.7 Hz, 2H), 3.691-3.57 (m, 2H), 3.49-3.41 (m, 2H), 3.36 (dd, J=14.1, 3.9 Hz, 1H), 3.08 (s, 3H), 2.99 (dd, J=14.2, 9.1 Hz, 1H), 2.30-2.10 (m, 1H), 1.44-1.34 (m, 1H), 1.25-1.04 (m, 12H).

¹³C NMR (151 MHz, CDCl₃) δ 173.8, 171.0, 158.1 (2C), 147.6, 137.2, 134.9 (d, J=3.2 Hz), 133.5 (d, J=10.1 Hz), 130.4 (d, J=12.5 Hz), 130.2, 127.5, 118.1 (d, J=85.7 Hz), 114.4, 111.6, 105.6, 66.1, 51.5, 49.2, 41.6, 37.7, 29.0, 28.9, 28.8, 28.7, 27.2, 26.3, 22.7, 22.4 (3C).

³¹P NMR (243 MHz, CDCl₃) δ 24.2.

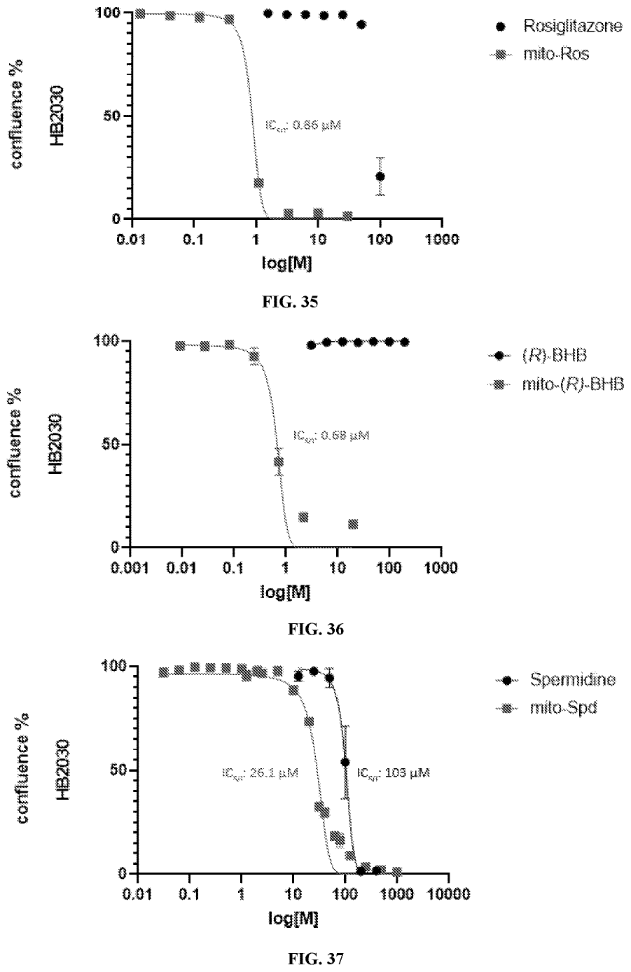

Results for the cell proliferation assay are shown in FIG. 35.

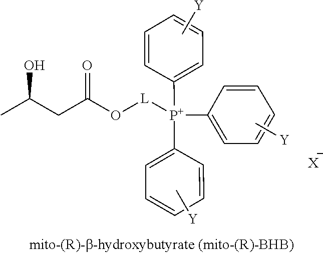

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, and ³¹P NMR (L=(CH₂)₁₀, Y═H)

¹H NMR (600 MHz, CDCl₃) δ 7.90-7.81 (m, 6H), 7.81-7.74 (m, 3H), 7.73-7.57 (m, 6H), 4.20-4.14 (m, 1H), 4.06 (td, J=6.7, 1.1 Hz, 2H), 3.84-3.75 (m, 2H), 3.11 (br, 1H), 2.51-2.36 (m, 2H), 1.65-1.39 (m, 6H), 1.37-0.94 (m, 13H).

¹³C NMR (151 MHz, CDCl₃) δ 173.0, 134.9 (d, J=3.2 Hz), 133.7 (d, J=10.1 Hz), 130.4 (d, J=12.5 Hz), 118.4 (d, J=85.6 Hz), 64.7, 64.2, 42.8, 29.2, 29.1, 29.0 (2C), 28.4, 25.7, 22.4.

³¹P NMR (243 MHz, CDCl₃) δ 24.4.

Results for the cell proliferation assay are shown in FIG. 36.

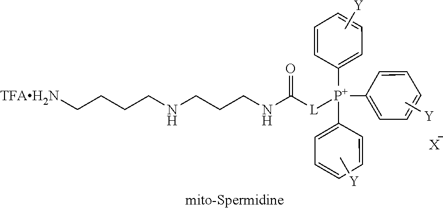

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, ³¹P NMR, and ¹⁹F NMR (L=(CH₂)₉, Y═H)

¹H NMR (600 MHz, DMSO-d₆) δ 7.92-7.86 (m, 3H), 7.83-7.72 (m, 12H), 3.60-3.49 (m, 2H), 3.04 (q, J=6.6 Hz, 2H), 2.58-2.51 (m, 2H), 2.48-2.39 (m, 4H), 2.01 (t, J=7.4 Hz, 2H), 1.56-1.40 (m, 8H), 1.40-1.32 (m, 4H), 1.31-1.13 (m, 8H).

¹³C NMR (151 MHz, DMSO-d₆) δ 172.4, 135.3 (d, J=3.0 Hz), 134.1 (d, J=10.1 Hz), 130.7 (d, J=12.4 Hz), 119.1 (d, J=85.6 Hz), 49.7, 47.3, 37.1, 35.9, 30.3, 30.2, 29.9, 29.1, 29.1 (2C), 28.5, 27.4, 25.8, 22.2, 22.1, 20.8, 20.4.

³¹P NMR (243 MHz, DMSO-d₆) δ 24.1.

¹⁹F NMR (565 MHz, DMSO-d₆) δ −73.4

Results for the cell proliferation assay are shown in FIG. 37.

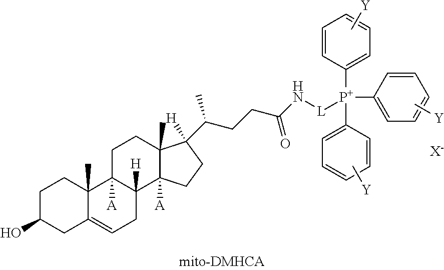

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, and ³¹P NMR (L=(CH₂)₁₀, Y═H)

¹H NMR (600 MHz, CDCl₃) δ 7.84-7.75 (m, 3H), 7.74-7.58 (m, 12H), 5.66 (s, 1H), 5.36-5.30 (m, 1H), 3.55-3.46 (m, 1H), 3.24-3.08 (m, 4H), 2.32-2.18 (m, 3H), 2.17 (s, 3H), 2.08-1.90 (m, 3H), 1.89-1.72 (m, 4H), 1.58-1.38 (m, 11H), 1.35-1.14 (m, 13H), 1.11-1.02 (m, 3H), 1.01-0.94 (m, 4H), 0.94-0.86 (m, 4H), 0.66 (s, 3H).

¹³C NMR (151 MHz, CDCl₃) δ 173.7, 140.7, 135.2 (d, J=3.1 Hz), 133.3 (d, J=9.9 Hz), 130.6 (d, J=12.5 Hz), 121.7, 118.0 (d, J=85.9 Hz), 71.8, 56.7, 55.8, 50.1, 42.3 (2C), 39.7, 39.4, 37.2, 36.5, 35.5, 33.6, 31.9 (2C), 31.6, 30.9, 29.3, 28.8 (2C), 28.6, 28.1, 26.6, 24.3, 21.1, 19.4, 18.4, 11.9.

³¹P NMR (243 MHz, CDCl₃) δ 23.7 (s, 1P), −144.3 (septet, J=704.7 Hz, 1P).

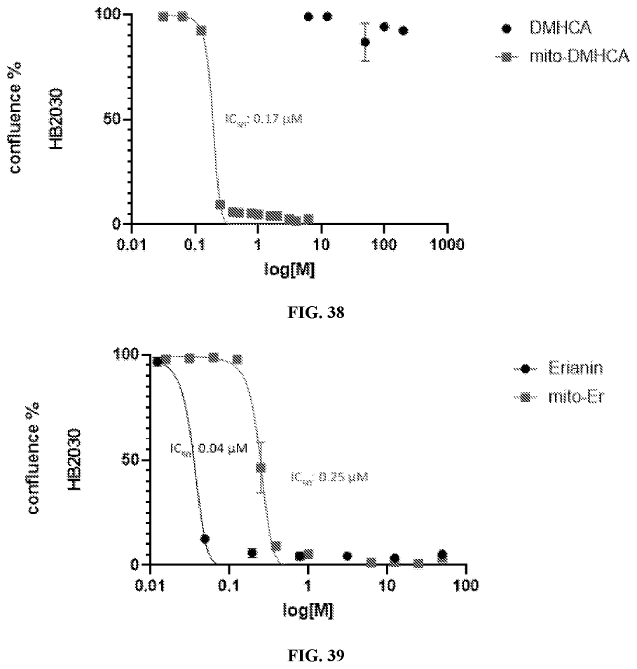

Results for the cell proliferation assay are shown in FIG. 38.

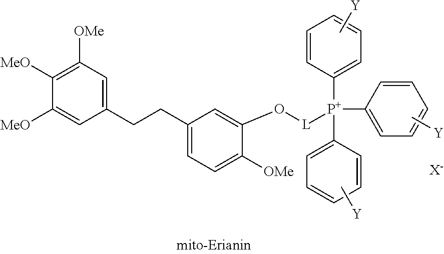

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, and ³¹P NMR (L=(CH₂)₁₀, Y═H)

¹H NMR (600 MHz, CDCl₃) δ 7.88-7.82 (m, 6H), 7.80-7.76 (m, 3H), 7.72-7.67 (m, 6H), 6.78 (d, J=8.0 Hz, 1H), 6.73-6.62 (m, 2H), 6.36 (s, 2H), 3.94 (t, J=6.9 Hz, 2H), 3.82 (s, 3H), 3.81 (s, 9H), 2.83-2.80 (m, 2H), 1.81-1.74 (m, 2H), 1.72-1.70 (m, 2H), 1.65-1.58 (m, 4H), 1.43-1.34 (m, 2H), 1.31-1.17 (m, 10H).

¹³C NMR (151 MHz, CDCl₃) δ 153.0, 148.4, 147.7, 137.6, 136.1, 134.9 (d, J=3.1 Hz), 134.3, 133.7 (d, J=9.9 Hz), 130.4 (d, J=12.5 Hz), 120.4, 118.5 (d, J=85.9 Hz), 113.7, 111.8, 105.4, 69.0, 60.8, 56.1, 56.0, 38.5, 37.5, 29.4, 29.3, 29.2, 29.1, 25.9, 22.9, 22.7, 22.6 (2C).

³¹P NMR (243 MHz, CDCl₃) δ 24.5.

Results for the cell proliferation assay are shown in FIG. 39.

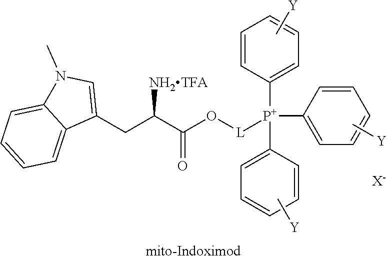

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, ³¹P NMR, and ¹⁹F NMR (L=(CH₂)₁₀, Y═H)

¹H NMR (600 MHz, DMSO-d₆) δ 8.49 (br, 2H), 7.92-7.87 (m, 3H), 7.84-7.73 (m, 12H), 7.48 (d, J=7.8 Hz, 1H), 7.39 (d, J=8.4 Hz, 1H), 7.19 (s, 1H), 7.14 (t, J=8.1 Hz, 1H), 7.03 (t, J=7.9 Hz, 1H), 4.22 (t, J=6.7 Hz, 1H), 4.04-3.94 (m, 2H), 3.73 (s, 3H), 3.59-3.50 (m, 2H), 3.27 (d, J=6.0 Hz, 1H), 3.21 (d, J=7.2 Hz, 1H), 1.56-1.47 (m, 2H), 1.47-1.34 (m, 4H), 1.29-1.20 (m, 2H), 1.19-1.09 (m, 6H), 1.09-1.00 (m, 2H).

¹³C NMR (151 MHz, DMSO-d₆) δ 169.9, 137.1, 135.4 (d, J=3.0 Hz), 134.1 (d, J=10.2 Hz), 130.7 (d, J=12.5 Hz), 129.5, 127.7, 121.8, 119.3, 118.9 (d, J=88.2 Hz), 118.8, 110.2, 106.3, 66.1, 53.2, 32.9, 29.2 (2C), 29.0, 28.6, 28.2, 26.7, 25.5, 22.3, 20.8, 20.5.

³¹P NMR (243 MHz, DMSO-d₆) δ 24.1.

¹⁹F NMR (565 MHz, DMSO-d₆) δ −73.6.

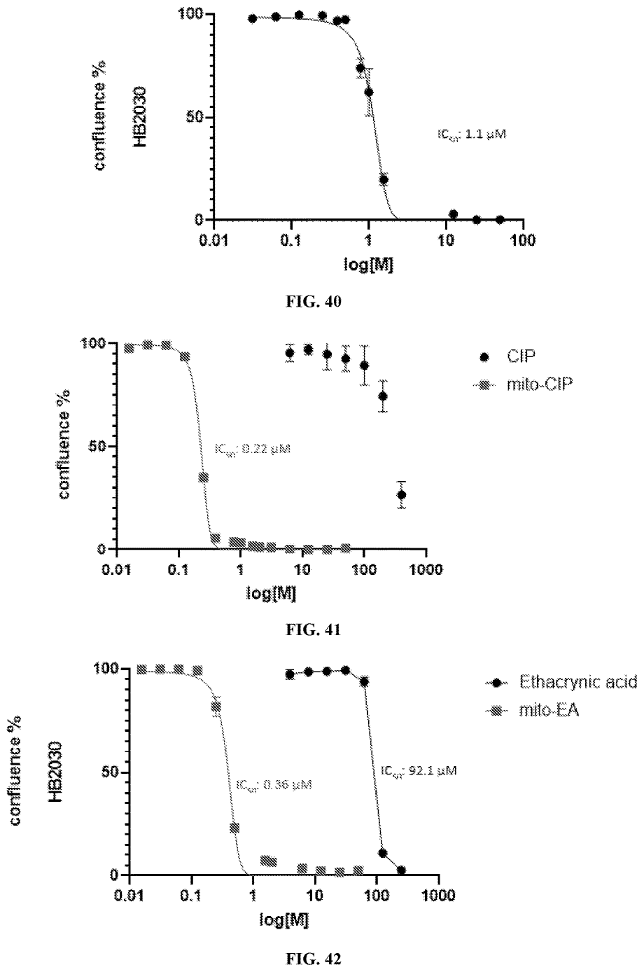

Results for the cell proliferation assay are shown in FIG. 40.

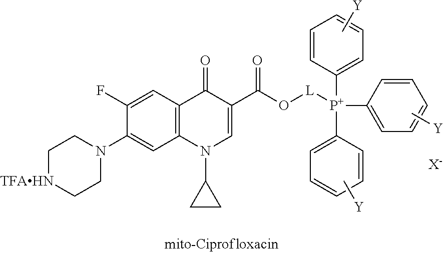

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, ³¹P NMR, and ¹⁹F NMR (L=(CH₂)₁₀, Y═H)

¹H NMR (600 MHz, CDCl₃) δ 8.50 (s, 1H), 8.01 (d, J=13.3 Hz, 1H), 7.82-7.74 (m, 9H), 7.72-7.66 (m, 6H), 7.28 (d, J=7.0 Hz, 1H), 4.28 (t, J=6.9 Hz, 2H), 3.24-3.21 (m, 3H), 3.09-3.06 (m, 3H), 1.75 (p, J=7.0 Hz, 2H), 1.62-1.50 (m, 4H), 1.40 (p, J=7.3 Hz, 2H), 1.34-1.17 (m, 15H), 1.14-1.07 (m, 2H).

¹³C NMR (151 MHz, CDCl₃) δ 173.2, 165.7, 154.3, 152.6, 148.0, 145.0 (d, J=10.4 Hz), 138.1, 135.0 (d, J=3.1 Hz), 133.5 (d, J=10.1 Hz), 130.5 (d, J=12.6 Hz), 118.4 (d, J=85.9 Hz), 113.2 (d, J=23.2 Hz), 110.4, 104.8 (d, J=2.7 Hz), 64.9, 51.2 (d, J=4.5 Hz), 45.9, 34.5, 29.3 (3C), 29.1, 29.0 (2C), 28.7, 25.9, 22.4 (d, J=4.7 Hz), 8.1.

³¹P NMR (243 MHz, CDCl₃) δ 24.2.

¹⁹F NMR (565 MHz, CDCl₃) δ −74.7 (s, 3F), −123.8 (d, J=7.0 Hz, 1F).

Results for the cell proliferation assay are shown in FIG. 41.

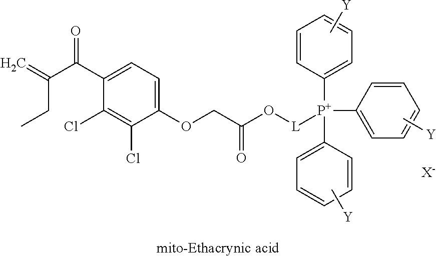

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, and ³¹P NMR (L=(CH₂)₁₀, Y═H)

¹H NMR (600 MHz, CDCl₃) δ 7.89-7.82 (m, 6H), 7.81-7.75 (m, 3H), 7.71-7.67 (m, 6H), 7.13 (d, J=8.5 Hz, 1H), 6.80 (d, J=8.5 Hz, 1H), 5.93 (t, J=1.5 Hz, 1H), 5.58 (d, J=1.0 Hz, 1H), 4.75 (s, 2H), 4.18 (t, J=6.7 Hz, 2H), 3.86-3.78 (m, 2H), 2.44 (q, J=7.4 Hz, 2H), 1.66-1.56 (m, 6H), 1.29-1.17 (m, 10H), 1.13 (t, J=7.5 Hz, 3H).

¹³C NMR (151 MHz, CDCl₃) δ 195.8, 167.8, 155.5, 150.1, 134.9 (d, J=3.1 Hz), 133.7 (d, J=10.1 Hz), 131.4, 130.4 (d, J=12.5 Hz), 128.7, 126.8, 123.3, 118.5 (d, J=86.3 Hz), 110.8, 66.3, 65.8, 45.2, 32.5, 29.2 (2C), 29.0, 28.9, 28.4, 26.7, 25.6, 23.4, 12.4.

³¹P NMR (243 MHz, CDCl₃) δ 24.5.

Results for the cell proliferation assay are shown in FIG. 42.

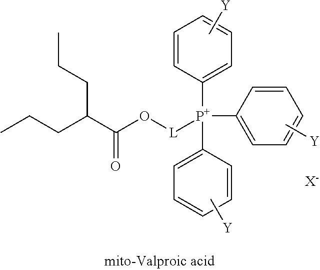

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, and ³¹P NMR (L=(CH₂)₁₀, Y═H)

¹H NMR (600 MHz, CDCl₃) δ 7.89-7.83 (m, 6H), 7.81-7.75 (m, 3H), 7.72-7.66 (m, 6H), 4.03 (t, J=6.7 Hz, 2H), 3.90-3.80 (m, 2H), 2.34 (tt, J=9.1, 5.3 Hz, 1H), 1.61-1.52 (m, 3H), 1.42-1.35 (m, 2H), 1.32-1.14 (m, 11H), 0.88 (t, J=7.3 Hz, 6H).

¹³C NMR (151 MHz, CDCl₃) δ 176.7, 134.9 (d, J=3.0 Hz), 133.7 (d, J=9.9 Hz), 130.4 (d, J=12.5 Hz), 118.5 (d, J=85.6 Hz), 64.1, 45.3, 34.7, 30.4 (d, J=15.6 Hz), 29.4, 29.2, 29.1 (d, J=3.5 Hz), 28.6, 25.8, 22.9, 22.7 (d, J=4.5 Hz), 22.6, 20.6, 14.0.

³¹P NMR (243 MHz, CDCl₃) δ 24.6.

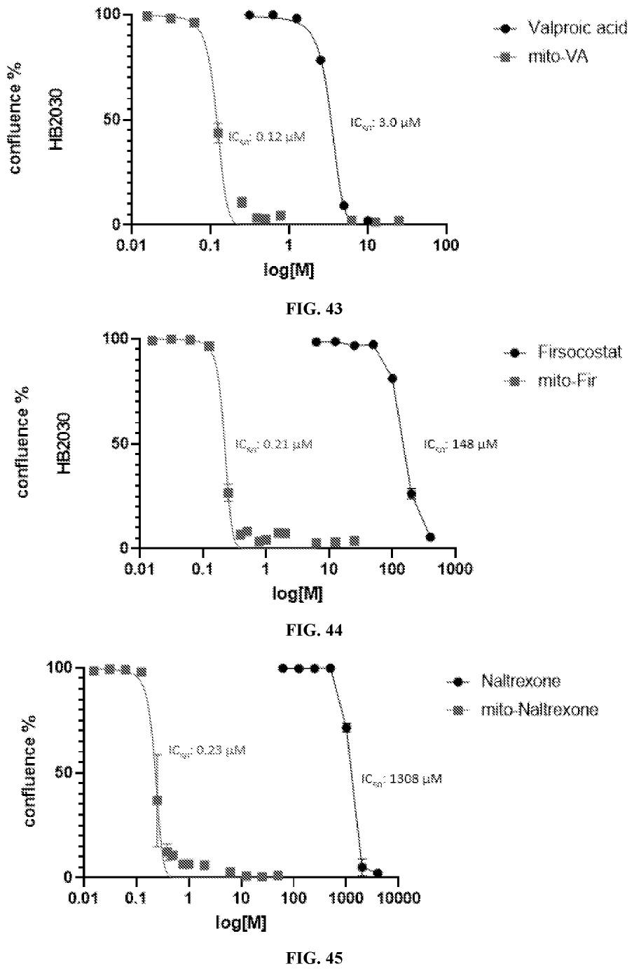

Results for the cell proliferation assay are shown in FIG. 43.

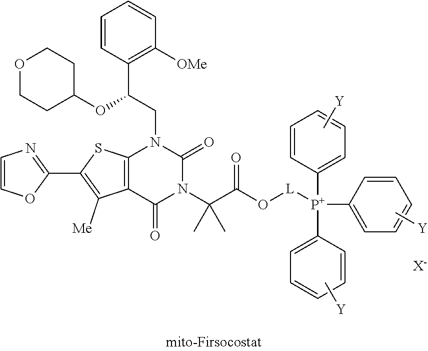

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, and ³¹P NMR (L=(CH₂)₁₀, Y═H)

¹H NMR (600 MHz, CDCl₃) δ 7.88-7.82 (m, 6H), 7.81-7.76 (m, 3H), 7.74 (d, J=0.8 Hz, 1H), 7.72-7.67 (m, 6H), 7.56 (dd, J=7.6, 1.7 Hz, 1H), 7.28 (ddd, J=8.1, 7.3, 1.7 Hz, 1H), 7.16 (d, J=0.8 Hz, 1H), 7.01 (td, J=7.5, 1.1 Hz, 1H), 6.85 (dd, J=8.3, 1.0 Hz, 1H), 5.39-5.34 (m, 1H), 4.15-4.01 (m, 3H), 3.85 (s, 3H), 3.85-3.77 (m, 2H), 3.75-3.63 (m, 2H), 3.52-3.44 (m, 1H), 3.44-3.39 (m, 1H), 2.81 (s, 3H), 1.80 (s, 3H), 1.77 (s, 3H), 1.74-1.66 (m, 6H), 1.69 (s, 4H), 1.60-1.52 (m, 4H), 1.26-1.08 (m, 10H).

¹³C NMR (151 MHz, CDCl₃) δ 173.4, 159.7, 157.6, 156.8, 155.3, 150.0, 138.3, 138.0, 134.9, 134.9, 133.7, 133.7, 130.5, 130.4, 129.3, 128.0, 127.3, 127.1, 120.9, 118.8, 118.2, 115.1, 114.7, 110.2, 77.2, 77.0, 76.8, 69.7, 65.2, 65.0, 63.6, 55.4, 33.2, 32.5, 31.3, 30.3 (d, J=15.6 Hz), 29.4, 29.1, 29.1, 28.4, 26.7, 25.9, 22.9, 22.6, 22.6, 22.5, 15.2, 14.8.

³¹P NMR (243 MHz, CDCl₃) δ 24.5.

Results for the cell proliferation assay are shown in FIG. 44.

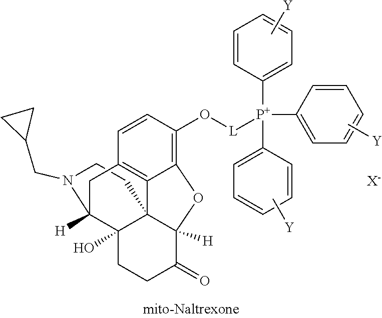

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
X is any halogen, TFA, triflate, hexafluorophosphate or acetic acid.

¹H NMR, ¹³C NMR, and ³¹P NMR (L=(CH₂)₁₀, Y═H)

¹H NMR (600 MHz, CDCl₃) δ 7.89-7.82 (m, 6H), 7.81-7.76 (m, 3H), 7.74-7.67 (m, 6H), 6.69 (d, J=8.2 Hz, 1H), 6.60 (d, J=8.3 Hz, 1H), 4.64 (s, 1H), 4.13-4.01 (m, 2H), 3.85-3.75 (m, 2H), 3.10-2.97 (m, 2H), 2.47-2.36 (m, 2H), 2.27 (dt, J=14.4, 3.1 Hz, 1H), 1.78-1.51 (m, 12H), 1.42-1.33 (m, 2H), 1.31-1.13 (m, 9H), 0.91-0.84 (m, 1H), 0.60-0.52 (m, 2H), 0.23-0.10 (m, 2H).

³¹P NMR (243 MHz, CDCl₃) δ 24.5.

Results for the cell proliferation assay are shown in FIG. 45.

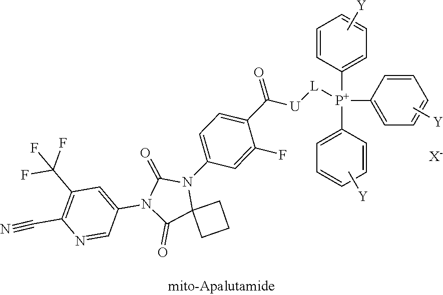

wherein
L is selected from an unsubstituted C₁-C₂₀ alkyl, substituted C₁-C₂₀ alkyl, C₂-C₂₀ alkenyl, C₁-C₂₀ alkynyl, phenyl, phenyl substituted C₁-C₂₀ alkyl, cycloalkyl substituted C₁-C₂₀ alkyl, PEG and substituted heterocycles.
Each Y is independently selected from —H, —OMe, —OCF₃, CF₃, or Cl, and can be located at any ortho, meta and para position.
U is O, or NH.
X is any halogen, TFA, triflate, hexafluorophosphate or acetic acid.

¹H NMR, ¹³C NMR, and ³¹P NMR (U═NH, L=(CH₂)₁₀, Y═H)

¹H NMR (600 MHz, CDCl₃) δ 9.14 (d, J=2.2 Hz, 1H), 8.44 (d, J=2.2 Hz, 1H), 8.15 (t, J=7.9 Hz, 1H), 7.88-7.81 (m, 6H), 7.81-7.74 (m, 3H), 7.71-7.64 (m, 6H), 7.36-7.30 (m, 2H), 4.36 (t, J=6.3 Hz, 2H), 3.88-3.80 (m, 2H), 2.81-2.73 (m, 2H), 2.62-2.54 (m, 2H), 2.29-2.20 (m, 1H), 1.78-1.66 (m, 3H), 1.64-1.57 (m, 3H), 1.47-1.38 (m, 2H), 1.35-1.13 (m, 9H).

³¹P NMR (243 MHz, CDCl₃) δ 24.5.

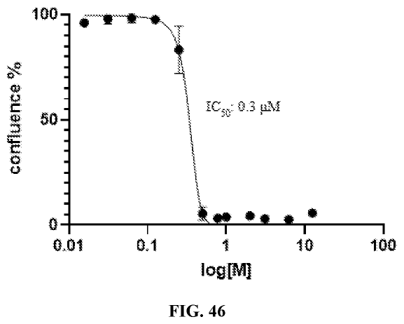

Results for the cell proliferation assay are shown in FIG. 46.

### Antiproliferative Effects of MTAs

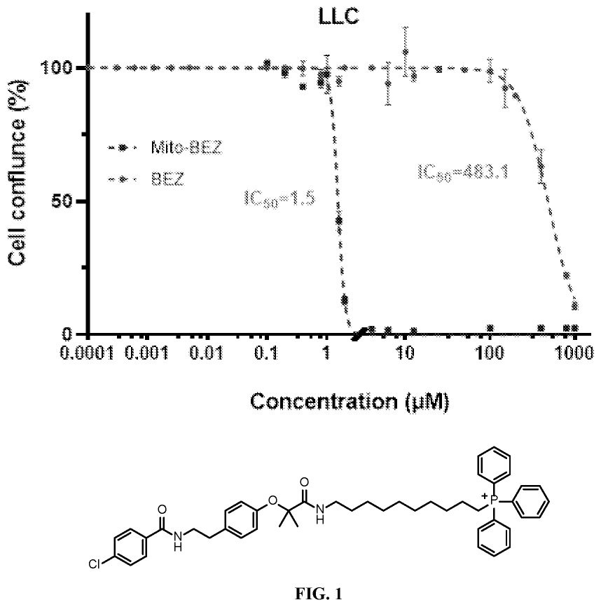

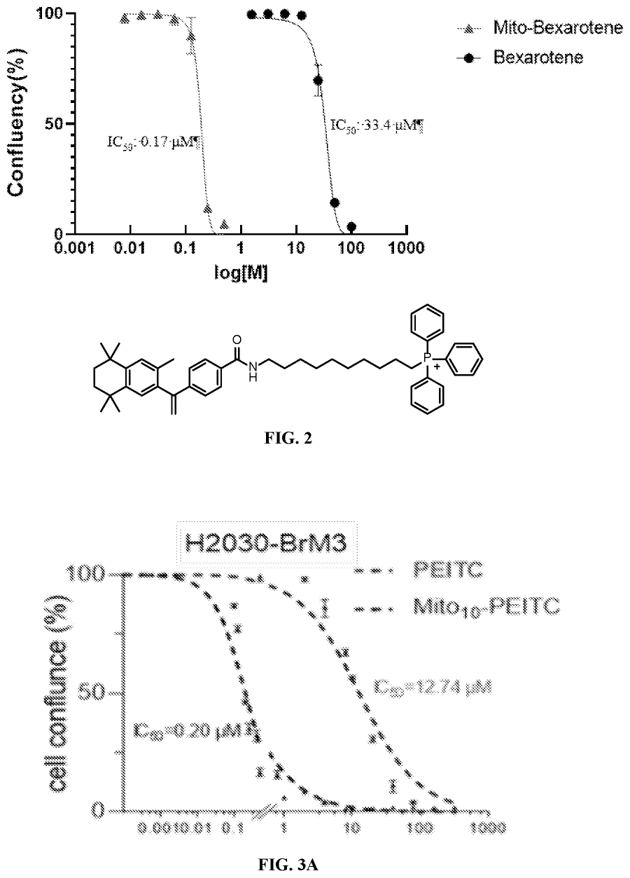

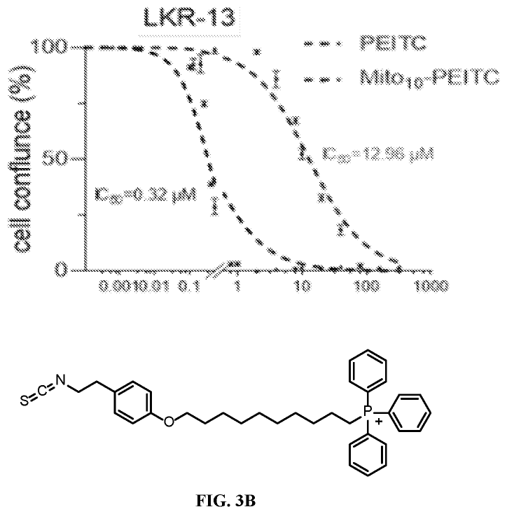

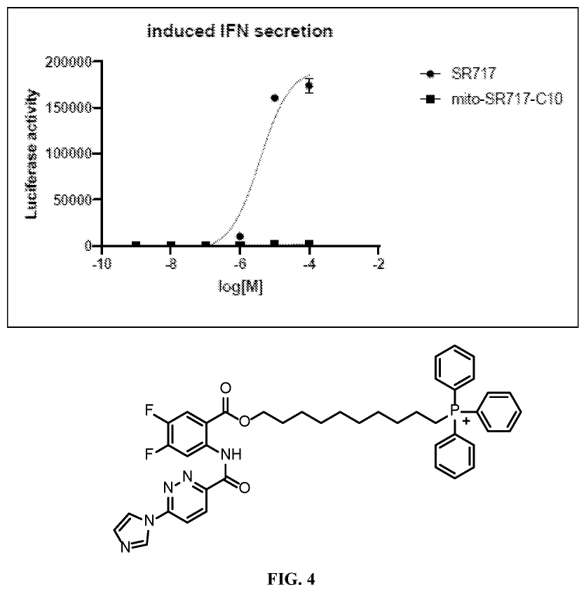

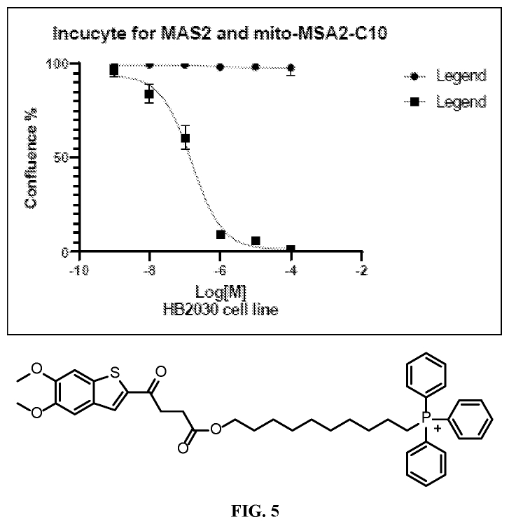

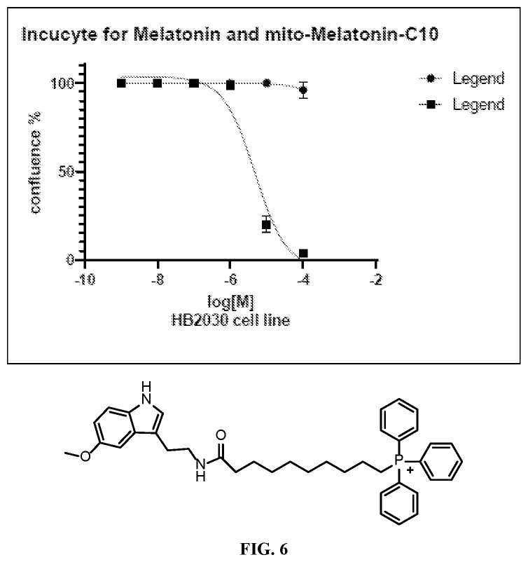

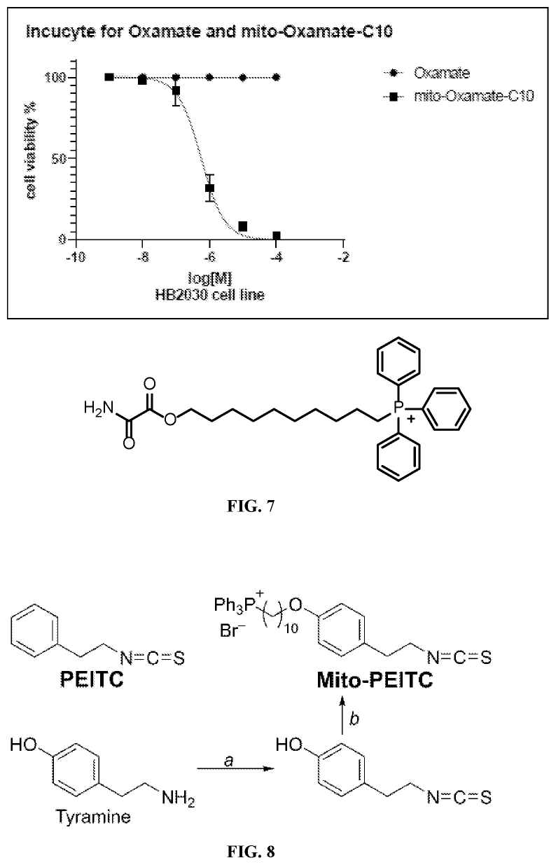

Experiments that have been performed to demonstrate the relative antiproliferative effects of selected (not all) MTAs in lung cancer cell lines. Results show that the potency of MTAs are much greater than their parental compounds. Results are shown in FIGS. 1-7.

### Antitumor Effects of MTAs in Animal Models

Synthesis, purification and structural characterization of PEITC mitochondria-targeted analogs. It was expected that linking PEITC to TPP⁺ via a long alkyl chain enhances its cellular and mitochondrial uptake. It has been demonstrated that selective accumulation of TPP⁺-linked bioactive compounds in cancer cell mitochondria, according to the Nernst equation. The alkyl chain in Mito-PEITC separates the bulky TPP⁺ group from the isothiocyanate group of PEITC, minimizing the effect of TPP⁺ on the pharmacophore. Mito-PEITC was synthesized by converting tyramine into 4-OH-PEITC, followed by its derivatization with bromodecyl-TPP⁺ (FIG. 8). The product was isolated on gel chromatography, purified by HPLC, and characterized by NMR and high-resolution mass spectrometry. Mito-PEITC purity from different synthesized batches and its stability in prepared stock solutions will be evaluated by HPLC analyses.

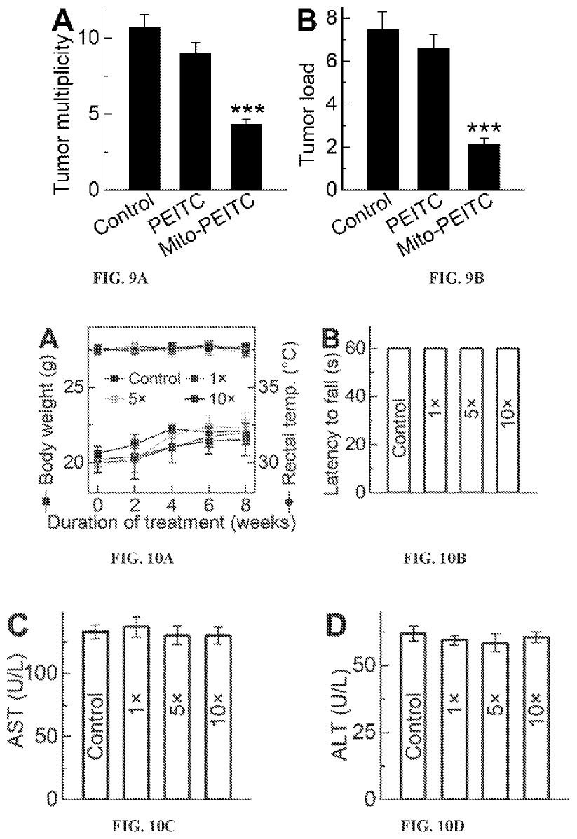

Efficacy of Mito-PEITC on lung tumorigenesis in A/J mice. PEITC has shown specific inhibitory effects on rodent tumor development induced by the tobacco-specific carcinogen NNK when administered during the initiation stage, but less efficacy when given in the post-carcinogen treatment period. Although PEITC is capable of inhibiting NNK-induced lung carcinogenesis, it is not effective against lung tumorigenesis induced by B(a)P. Mito-PEITC inhibited B(a)P-induced lung tumor development in A/J mice in a post-initiation model (4 μmol/kg, 5 days/week, 20 weeks) given one week after B(a)P (FIG. 9A-9B), while PEITC was ineffective at 25 times higher dose (100 μmol/kg, 5 days/week, 20 weeks). Thus, Mito-PEITC, unlike its parental compound PEITC, will prevent all stages of progression of B(a)P-induced lung tumors in A/J mice with a favorable safety profile.

Mito-PEITC did not cause any toxicities at doses up to 10 times higher than the effective dose (ED). An 8-week toxicology study to evaluate potential toxicities of Mito-PEITC in A/J mice, especially on neural and muscle cells using a Modified Irwin Screen, developed as a comprehensive observational battery to screen for central nervous system (CNS) effects of agents. Modifications of this test are extensively done in the pharmaceutical industry and in academic research to identify changes in neurological function as a result of agent intoxication, neurotoxicity, or genetic manipulation. The screen used here employs 35 distinct measurements to assess sensorimotor, neurological, and autonomic nervous system function. The ED of Mito-PEITC is 4 μmol/kg. No significant differences between control and Mito-PEITC treated mice at doses up to 10×ED in A/J mice (FIG. 10A-10D) on any of the 35 metrics tested (detecting sensorimotor, neurologic, motor, and autonomic nervous system dysfunction) over 8-weeks of treatment was observed. No toxic effects were seen in either neural (frontal cortex and cerebellum) or muscles (skeletal muscles including the soleus, plantaris, gastrocnemius, tibialis anterior and quadriceps) by histopathology. Thus, Mito-PEITC, at doses up to 10×ED, did not elicit any toxicities.

Prevention of lung cancer brain metastasis by Mito-PEITC. Brain metastasis is one of the most intractable clinical problems of lung cancer and a major cause of lung cancer mortality. About 10% of patients have brain metastasis at the time of diagnosis, and 40%-50% develop brain metastasis during a course of lung cancer. Halting the dissemination and metastasis of tumor cells is critical to reducing lung cancer mortality. The data show that Mito-PEITC has a significantly enhanced efficacy against brain metastasis of lung cancer cells (FIGS. 11A-11C).

Mito-PEITC was found to inhibit LUAD brain metastasis in Nod/Scid mice with cardiac injection of luciferase (luc)-expressing human LUAD H2030-BrM3 cells. To ensure the accuracy of injection and precision in the number of cells injected, high-resolution echocardiography was used to guide the needle into the left ventricle (FIG. 11A), where the cells were released. Mice were imaged for luciferase expression as a marker of tumor growth, using an IVIS 100 imaging system, over five weeks after cell injection. Regions of interest were defined manually, and LivingImage and Igor software was used to determine total photon flux. One day after engrafting LUAD cells, PEITC (100 mol/kg) or Mito-PEITC (4 mol/kg) were administered by daily oral gavage. While PEITC was ineffective at this dose, Mito-PEITC accumulated in the brain tissue (FIG. 11C) and inhibited LUAD brain metastasis, indicating that Mito-PEITC can suppress the growth of brain metastasis (FIGS. 11A-11C).

Molecular mechanisms of action for PEITC. PEITC has been shown to inhibit carcinogenesis, inflammation, invasion and metastasis by targeting multiple cell growth and survival-related signaling pathways in cancer cells. PEITC is known to prevent the initiation phase of carcinogenesis by direct function on both phase I and phase II drug metabolizing enzymes leading to the reduced chemical carcinogens. PEITC inhibits the progression of tumorigenesis primarily through the modulation of mitochondrial and redox pathways. There is accumulating evidence that PEITC inhibits cancer cell growth and invasion through a mitochondrial mechanism. PEITC disrupts mitochondrial complex I and III, depletes mitochondrial GSH and leads to increased hydrogen peroxide (H₂O₂) levels and oxidation of mitochondrial peroxiredoxin-3 (Prx3), resulting in redox modulation of several cellular processes. This ultimately downregulates redox-sensitive proteins, such as STAT3 (signal transducer and activator of transcription 3), a major downstream mediator of these pathways. STAT3 is an oncogene regulated by receptor tyrosine kinases, G-protein coupled receptors and interleukin families via phosphorylation. Phosphorylated STAT3 undergoes dimerization and trans-localization to either the nucleus or mitochondria to promote enhanced cell proliferation, survival, and invasion for many cancers. The data show that the anti-proliferative and anti-invasive effects of PEITC involve inhibition of mitochondrial respiration and the downstream STAT3 activities. Specifically, PEITC inhibits complex I and complex III activities in cancer cells, induces redox stress, promotes oxidized Prx3, and inhibits mitochondrial STAT3 phosphorylation (p-STAT3⁷²⁷) in LUAD cells.

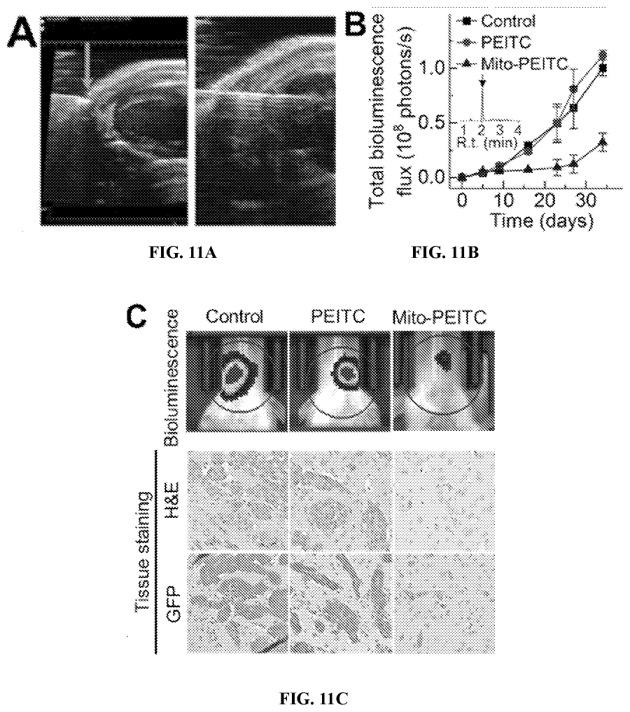

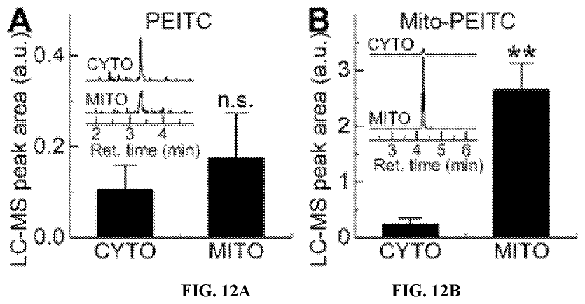

Thus, Mito-PEITC will amplify the significance of targeting mitochondrial redox status and bioenergetics as key its mechanism of action. Indeed, the data show that Mito-PEITC preferentially accumulates within cancer cell mitochondria versus normal cells (FIG. 12A-12B), leading to significantly enhanced cancer preventive effects with little or no toxicity to normal cells. Finally, Mito-PEITC is capable of enhancing the anti-tumor activity of PD-1 blockade in mouse models.

### Example 2: Targeting Phenethyl Isothiocyanate to Mitochondria Reprograms Cancer and Immune Cells in the Tumor Microenvironment

Phenethyl isothiocyanate (PEITC), a naturally occurring compound present in cruciferous vegetables, has been shown to inhibit oxidative phosphorylation and induce cancer cell apoptosis. PEITC achieves these activities through a mitochondria-dependent mechanism and ROS formation, suggesting potential roles for mitochondrial bioenergetic function and redox homeostasis in oncogenesis. Given this rationale, the specific role of mitochondrial function and redox status in lung cancer development was study by conjugating PEITC to a targeting agent that drives it into mitochondria and increases its efficacy. A mitochondria-targeted form was developed of PEITC (Mito-PEITC) to facilitate its mitochondrial accumulation and tested its efficacy in a lung cancer model. Mito-PEITC has dramatically greater potency and efficacy against highly metastatic lung cancer cell lines than PEITC in vitro, as well as in orthotopic lung tumor syngrafts and brain metastases in vivo. Mito-PEITC inhibits cell proliferation, mitochondrial complexes I & III, and glycolysis; stimulates reactive oxygen species generation; oxidizes mitochondrial peroxiredoxin-3; and suppresses mitoSTAT3 phosphorylation, leading to cancer cell death via enhanced apoptosis. Furthermore, Mito-PEITC triggered potent T cell immune responses and prevented brain metastasis and lung metastasis. The impact of Mito-PEITC on the immune cells in the tumor microenvironment (TME) was investigated. Mito-PEITC treatment led to significant decreases in both granulocytic myeloid-derived suppressor cells (G-MDSCs) and regulatory T cells (Tregs) in the TME. Concomitantly, an increase in tumor infiltrating CD4⁺ and CD8⁺ T cells was observed. Mito-PEITC treatment also improved the antitumor activity of immunotherapy via the PD-1 blockade. These findings implicate G-MDSCs and Tregs as targets of Mito-PEITC that augment its antitumor efficacy. The results show that Mito-PEITC, with its favorable toxicity profile, exhibited a striking inhibitory effect on lung cancer progression and metastasis by targeting a fundamental difference in metabolic plasticity between cancer cells and effector T cells in the TME.

PEITC, a key bioactive compound found in cruciferous vegetables, has shown potent antitumor effects in both in vitro and in vivo preclinical models and in human trials (Bayoumy, K., et al., Cancer Prevention Research, 2022. 15(3): p. 139-141; Yuan, J.-M., et al., Cancer Prevention Research, 2016. 9(5): p. 396-405 (“Yuan, et al.”); Soundararajan, P. and J. S. Kim, Molecules, 2018. 23(11): p. 2983; Palliyaguru, D. L., et al., Mol Nutr Food Res, 2018. 62(18): p. e1700965). The pharmacokinetics of PEITC have been extensively studied in rodents and humans (Conaway, C. C., et al., Drug Metab Dispos, 1999. 27(1): p. 13-20 (“Conaway, et al.”); Ji, Y., et al., Pharm Res, 2005. 22(10): p. 1658-66; Morris, M. E. and R. A. Dave, Aaps j, 2014. 16(4): p. 705-13 (“Morris, et al.”); Konsue, N., et al., Mol Nutr Food Res, 2010. 54(3): p. 426-32). The major pharmacokinetic properties of PEITC include linear and first-order absorption, high plasma protein binding, capacity-limited tissue distribution, reversible metabolism, and hepatic elimination (Morris, et al.). PEITC accumulates in several organs, including the lungs, and it crosses the blood-brain barrier to accumulate in the brains of rodents. Using GST-catalyzed conjugations of PEITC with glutathione (GSH), the liver has been demonstrated as the major route of metabolism for PEITC (Zhang, Y., et al., Biochem Biophys Res Commun, 1995. 206(2): p. 748-55; Kolm, R. H., et al., Biochem J, 1995. 311 (Pt 2)(Pt 2): p. 453-9; Meyer, D. J., et a., Biochem J, 1995. 306 (Pt 2)(Pt 2): p. 565-9). GSH adducts are further metabolized to mercapturic acid derivatives via the actions of cysteinylglycinase and acetyltransferase (Morris, et al.; Ji, Y. and M. E. Morris, Anal Biochem, 2003. 323(1): p. 39-47). PEITC has shown extremely low toxicity for female rats (Liu, H., et al., Wei Sheng Yan Jiu, 2011. 40(3): p. 283-6). Finally, a recent clinical trial reveals that PEITC was safe and inhibited carcinogen metabolism (Yuan, et al.).

PEITC has been shown to inhibit carcinogenesis, inflammation, invasion, and metastasis by targeting multiple cell growth and survival-related signaling pathways in cancer cells (Conaway, et al.; Gupta, P., et al., PLOS ONE, 2013. 8(6): p. e67278). PEITC is known to prevent the initiation phase of carcinogenesis by acting directly on both phase I and phase II drug-metabolizing enzymes, leading to reduced amounts of chemical carcinogens (Hecht, S. S., J Nutr, 1999. 129(3): p. 768s-774s; Hecht, S. S., Drug Metab Rev, 2000. 32(3-4): p. 395-411; Hecht, S. S., et al., Nat Rev Cancer, 2009. 9(7): p. 476-88). PEITC inhibits the progression of tumorigenesis primarily by modulating mitochondrial and reduction-oxidation (redox) pathways (Brown, K. K., et al., FEBS Letters, 2010. 584(6): p. 1257-1262 (“Brown, et al.”); Chen, G., et al., Antioxid Redox Signal, 2011. 15(12): p. 2911-21 (“Chen, et al.”); Jutooru, I., et al., Molecular and Cellular Biology, 2014. 34(13): p. 2382-2395 (“Jutooru, et al.”); Trachootham, D., et al., Cancer Cell, 2006. 10(3): p. 241-52 (“Trachootham, et al.”)). Several studies have demonstrated mitochondrial mechanisms of inhibition of cancer cell growth and invasion by PEITC (Brown, et al., Chen, et al., Jutooru, et al., Trachootham, et al., and Gupta, P., et al., Biochimica et Biophysica Acta (BBA)
Reviews on Cancer, 2014. 1846(2): p. 405-424). First, PEITC disrupts mitochondrial complex I and III, and depletes mitochondrial GSH, leading to increased levels of hydrogen peroxide (H₂O₂) and oxidized mitochondrial peroxiredoxin-3 (Prx3). These changes result in redox modulation of several cellular processes. This ultimately downregulates redox-sensitive proteins, such as STAT3 (signal transducer and activator of transcription 3), a known oncogene. STAT3 is regulated by receptor tyrosine kinases, G-protein coupled receptors, and interleukin families via phosphorylation. When phosphorylated, STAT3 dimerizes and translocates to either the nucleus or the mitochondria to promote enhanced cell proliferation, survival, and invasion for many cancers (Gong, A., et al., Mol Nutr Food Res, 2009. 53(7): p. 878-86; Wang, H., et al., Mol Carcinog, 2018. 57(4): p. 522-535; Shao, W. Y., et al., Cancer Biol Ther, 2017. 18(1): p. 26-35).

Agents that target cancer cell bioenergetics hold exciting promise for inhibiting tumor growth because they are less toxic to normal cells (Cheng, G., et al., Cancer research, 2012. 72(10): p. 2634-44 (“Cheng, et al., 2012”); Cheng, G., et al., British journal of cancer, 2014(May): p. 1-9 (“Cheng et al., 2014”)). Cancer cells have increased negative plasma membrane and mitochondrial transmembrane potentials. For example, the mitochondrial membrane potential in cancer cells (−220 mV) is more negative than that in normal cells (−140 mV). This discrepancy can drive a 10- to 100-fold higher uptake of cations to cancer cells (Biswas, S. K., 2015. 43(3): p. 435-49 (“Biswas, et al.”); Kalyanaraman, B., Redox Biol, 2020. 36: p. 101665 (“Kalyanaraman, et al.”); Huang, M., et al., Cancer Prev Res (Phila), 2021. 14(3): p. 285-306 (“Huang, et al.”)). As a result, delocalized lipophilic cations (DLC), such as triphenylphosphonium cation (TPP⁺), can selectively accumulate in cancer cells (Zielonka, J., et al., Chem Rev, 2017. 117(15): p. 10043-10120 (“Zielonka, et al.”); Modica-Napolitano, J. S. and J. R. Aprille, Adv Drug Deliv Rev, 2001. 49(1-2): p. 63-70; Kalyanaraman, B., et al., Redox Biol, 2018. 14: p. 316-327). Thus, conjugating TPP⁺ or other DLCs to compounds of interest has been an effective approach for targeting bioactive compounds to the mitochondria (Biswas, et al., Kalyanaraman, et al., Huang, et al, Zielonka, et al).

This strategy has already demonstrated promise in several studies. Although tumor cells rely to a large extent on aerobic glycolysis to generate ATP (the Warburg effect), mitochondria are indeed functional in most tumor cells (Cheng, et al., 2012; Cheng et al., 2014; Cheng, G., et al., Cancer Lett, 2015. 365(1): p. 96-106 (“Cheng, et al., 2015”); Weinberg, F., et al., Proc Natl Acad Sci USA, 2010. 107(19): p. 8788-93; Moreno-Sinchez, R., et al., Arch Biochem Biophys, 2023. 739: p. 109559). Several mitochondria-targeted, TPP⁺-conjugated, bioactive molecules (e.g., Mito-Q) decrease ATP levels more potently in cancer cells than in normal cells and can inhibit cancer cell proliferation at nontoxic submicromolar levels (Cheng, et al., 2012; Cheng, et al., 2014; Zielonka, et al.; Cheng, G., et al., BMC cancer, 2013. 13: p. 285-285 (“Cheng., et al., 2013”); Rao, V. A., et al., Journal of Biological Chemistry, 2010. 285(45): p. 34447-34459). Detailed profiling of cellular bioenergetics has recently provided insights on the intermediacy of mitochondrial metabolism in tumor cells (Cheng, et al., 2014; Barbi de Moura, M., et al., PLoS One, 2012. 7(8): p. e40690). Thus, targeting mitochondrial bioenergetics is emerging as an effective and viable preventive approach to inhibit the proliferation of tumor cells (Cheng, et al., 2012; Cheng, et al., 2014; Cheng, et al., 2015; and Cheng, et al., 2013). It has also been found that mitochondria-targeted agents have activity on subtypes of immune cells in the tumor microenvironment (TME) (Biswas, et al.; Kalyanaraman, et al.; Huang, et al.; Loftus, R. M., et al., J Biol Chem, 2016. 291(1): p. 1-10; Andrejeva, G., et al., Cell Metab, 2017. 26(1): p. 49-70; Li, X., et al., Nat Rev Clin Oncol, 2019. 16(7): p. 425-441; Gaber, T., et al., Nat Rev Rheumatol, 2017. 13(5): p. 267-279).

In this study, a mitochondria-targeted PEITC (Mito-PEITC) was developed by attaching the bulky TPP⁺ group to PEITC via a long alkyl chain, which separates TPP⁺ from PEITC and increases its lipophilicity and mitochondrial uptake in cells. The greater uptake of the TPP⁺-linked PEITC to tumor mitochondria leads to a greater effect on inhibiting mitochondrial complexes I & III and the downstream cascade. It was found that Mito-PEITC: (i) inhibits both complex I and complex III, (ii) increases mitochondrial oxidants, (iii) oxidizes peroxiredoxin-3, (iv) activates AMPK, and (v) inhibits STAT3^{ser727} phosphorylation and cell proliferation much more robustly than PEITC using both in vitro and in vivo models. Importantly, toxicology studies of Mito-PEITC revealed no toxicities over eight-week treatment of A/J mice, even at a dose 10-fold higher than the effective dose. The effects of Mito-PEITC on immune cells within the TME were also explored. It was found that Mito-PEITC prevented brain metastasis and lung metastasis. Mito-PEITC also improved the antitumor activity of immunotherapy that targets the PD-1 blockade.

Through flow cytometry, it was found that Mito-PEITC treatment decreased granulocytic myeloid-derived suppressor cells (g-MDSCs) and T_{regs} and increased CD4⁺ and CD8⁺ T cells. The results show that Mito-PEITC, with a favorable toxicity profile, exhibited a striking inhibitory effect on lung cancer progression and metastasis by targeting the difference in metabolic plasticity between cancer cells and effector T cells in the tumor microenvironment. Mito-PEITC is a potent, chemopreventive agent of lung tumor progression and metastasis that acts primarily through mitochondrial mechanisms.

### Experimental

### Cell Lines and Animals

LKR13 cells, which is a mouse lung adenocarcinoma line that expresses mutant KrasG12D on the SV129 background, were a generous gift from Dr. Jonathan M. Kurie (MD Anderson). LKR13-luc cells were generated by transfecting LKR13 cells with CMV-firefly luciferase lentivirus (Cellomics Technology). H2030-BrM3 and PC9-BrM3 cells were generously provided by Dr. Joan Massague (Cancer Biology and Genetics Program, Memorial Sloan Kettering Cancer Center, New York, NY). The H2030-BrM3 cells consistently form brain metastases in 100% of animals and were engineered to express a green fluorescent protein (GFP)-luciferase fusion protein, which enables monitoring of in vivo tumor growth and metastasis. LKR13 cells and H2030-BrM3 cells were cultured in complete medium consisting of RPMI-1640 (Thermo-Fisher) supplemented with 10% fetal bovine serum (Sigma) and 1% penicillin/streptomycin (Gibco). LKR13-luc cells were cultured in complete medium supplemented with geneticin (500 μg/mL) for the selection of neomycin-resistant cell lines, indicating maintenance of the transfected luciferase gene. NHBE cells were purchased from Lonza and cultured in BEGM bronchial epithelial cell growth medium (Lonza). B16 and B16 ρ0 cells were provided by Dr. Martina Bajzikova (Czech Academy of Sciences, Czech Republic) and cultured in DMEM (11965-092, Gibco) supplemented with 10% FBS, sodium pyruvate (1 mM) and uridine (50 μg/ml). All cells were kept frozen in liquid nitrogen and were used within 20 passages after thawing. All cell lines used in this study were authenticated and verified to be free of Mycoplasma contamination (Universal Mycoplasma Detection Kit, ATCC). Vinyl carbamate (VC) was purchased from Santa Cruz Biotechnology.

Female NOD/SCID mice, and female A/J mice were purchased from The Jackson Laboratory. All studies on animals were approved by the Houston Methodist Research Institute Institutional Animal Care and Use Committee (approval number: IS00006363).

HPLC analyses of hydroethidine and its oxidation products HPLC-based measurements of HE and its oxidation products were performed using an Agilent 1100 HPLC system (Santa Clara, CA) equipped with absorption and fluorescence detectors and a refrigerated autosampler (4° C.). The samples (50 μL) were injected into a reverse-phase column (Phenomenex, Kinetex C18, 100 mm×4.6 mm, 2.6 μm) equilibrated with 20% acetonitrile (MeCN), 80% water containing 0.1% trifluoroacetic acid. The compounds were eluted by increasing the content of MeCN from 20 to 56% over 4.5 min at a flow rate of 1.5 mL/min. The detection parameters were as previously reported (Cheng, G., et al., J Biol Chem, 2018. 293(26): p. 10363-10380). The standards of the oxidation products were prepared as described previously and the concentrations were normalized to protein level of the lysates (Zielonka, J., et al., Free Radic Biol Med, 2009. 46(3): p. 329-38; Zielonka, J., et al., Nat Protoc, 2008. 3(1): p. 8-21; Zielonka, J., et al., Methods Mol Biol, 2019. 1982: p. 243-258).

### Lysate Collections and Western Blot Analyses

H2030-BrM3 cells were seeded in 6-well plates and adhered overnight prior to treatment with 1 μM Mito-PEITC dissolved in dimethyl sulfoxide (DMSO) or vehicle control in cell line-specific medium. Cell lysates were prepared from cells harvested at 0, 6, 24, and 48 h post-treatment using lysis buffer (1% Triton X-100, 50 mM HEPES, pH 7.4, 150 mM NaCl, 1.5 mM MgCl₂, 1 mM EGTA, 100 mM NaF, 10 mM sodium pyrophosphate, 1 mM sodium orthovanadate, 10% glycerol) with complete EDTA-free protease and PhosSTOP phosphatase inhibitors (Sigma-Aldrich). Protein was quantified using the DC protein assay (Bio-Rad, Hercules, CA). Approximately 20 μg of protein was loaded in precast 4-20% Mini-Protean TGX gels (Bio-Rad), run for 1 h, transferred to a PVDF membrane with the Trans-Blot® Turbo™ system (Bio-Rad) for 30 min, blocked for 1 h at room temperature, incubated overnight with primary antibodies, and incubated with the secondary antibody for 1 h. Images were captured via the ChemiDoc Molecular Imager and bands were quantified with ImageLab analysis software (both from Bio-Rad). Expression levels were determined by chemiluminescent immunodetection and normalized to appropriate loading controls.

### Cellular Proliferation Assays

Cells were plated in 96-well tissue culture plates at 1,000-3,000 cells per well, treated with Mito-PEITC or vehicle control (DMSO), and cell proliferation was measured using a label-free, noninvasive cellular confluence assay (IncuCyte Live Cell Imaging Systems, IncuCyte FLR, Essen Bioscience, Ann Arbor, MI) as recommended by the manufacturer.

### Mitochondrial Function in Intact and Permeabilized Cells

Mitochondrial function was monitored using a Seahorse XF96 Extracellular Flux Analyzer (Agilent, Santa Clara, CA) (Weinberg, F., et al., Proc Natl Acad Sci USA, 2010. 107(19): p. 8788-93). Activities of mitochondrial respiratory complexes in permeabilized cells were measured according to the manufacturer's instructions. Briefly, after treatment, intact cells were permeabilized using 1 nmol/L Plasma Membrane Permeabilizer (PMP, Agilent) immediately prior to measuring the oxygen consumption rate (OCR). The oxygen consumption derived from mitochondrial complexes was measured using different mitochondrial substrates (e.g., pyruvate/malate for complex I and succinate for complex II). Rotenone, malonate and antimycin A were used as specific inhibitors of mitochondrial complexes I, II, and III, respectively. Mitochondrial function in intact B16 and B16 ρ0 cells was measured using the same methodology, but without cell permeabilization or treatment. During the assay, OCR was measured before and after consecutive injections of oligomycin (mitochondrial complex V inhibitor), FCCP (mitochondrial uncoupler) and rotenone+antimycin A.

### Redox Blots for Peroxiredoxins

The redox status of cytosolic and mitochondrial peroxiredoxins (Prx1 and Prx3, respectively) was determined by redox western blotting as previously described (Zhang, Q., et al., Cell Commun Signal, 2020. 18(1): p. 58). Briefly, after treatment, cells were washed quickly with HBSS and overlayed with a thiol-blocking buffer containing 0.1 MN-ethylmaleimide (NEM), 50 mM NaCl, 40 mM HEPES, pH 7.4, 1 mM PMSF, 1 mM EDTA, 1 mM EGTA, and 10 μg/mL catalase. Cells were harvested and incubated at room temperature for 15 min, pelleted (5 min, 800×g), and then the pellet was lysed in a small volume of the thiol blocking buffer supplemented with 1% CHAPS (3-((3-cholamidopropyl)dimethyl-ammonio)-1-propanesulfonate). Lysates were stored at −80° C. until analysis. On the day of analysis, the lysates were thawed on ice and then centrifuged for 5 min (8000×g, 4° C.). The supernatant fractions were subjected to nonreducing SDS-PAGE. The blots were probed with anti-Prx1 (sc-7381; 1:500) and anti-Prx3 (sc-59661; 1:500) antibodies (Santa Cruz Biotechnology), followed by HRP-conjugated secondary antibodies. After chemiluminescence measurements were taken using a luminescence imager, the blots were stripped and probed for β-actin as an indicator of protein load.

### Quantitation of Mitochondrial and Cytosolic PEITC and Mito-PEITC by LC-MS/MS

Cells were grown in 15-cm dishes and incubated with the compounds for 24 h in complete medium. After 24-h treatment, cells were washed twice with ice-cold-DPBS, harvested, and cell pellet stored at −80° C. until fractionation and extraction. Liquid chromatography-mass spectrometry (LC-MS/MS) analyses were performed on cell extracts using Shimadzu Nexera2 UHPLC system equipped with UV-Vis absorption and triple quadrupole mass spectrometry (LC-MS8030) detectors. Detection of the thiourea derivative of PEITC was accomplished using Raptor Biphenyl column (Restek, 100 mm×2.1 mm, 2.7 μm) equilibrated with mobile phase containing 90% water, 10% acetonitrile (MeCN), 0.1% formic acid. The analytes were eluted by increasing the content of MeCN from 10 to 50% over 5 min and detected in the multiple reaction monitoring (MRM) mode using the transition of 181.0>105.05 (positive mode). Mito-PEITC thiourea derivative was analyzed using the same column as used for PEITC but equilibrated with a mobile phase containing water (80%), MeCN (20%) and formic acid (0.1%). Mito-PEITC-thiourea was eluted by increasing the content of MeCN in the mobile phase from 20 to 100% over 6 min and detected in the MRM mode using the transition of 597.20>538.20 (positive mode). For both PEITC and Mito-PEITC analyses, electrospray ionization mode was used, and the mobile phase flow rate was set at 0.5 mL/min. For the LC-MS/MS-based monitoring of the reaction of Mito-PEITC with GSH, the same method as for Mito-PEITC thiourea was used, but the reaction mixtures were directly injected for analysis, without derivatization with NH₃. The MRM detection parameters for underivatized Mito-PEITC and its GSH adduct were 580.20>262.00 and 444.30>328.00, respectively, both in the positive mode. GSH adduct was detected as a double charged species (z=2+).

### Transwell Invasion Assay

Boyden chamber transwells pre-coated with growth-factor-reduced Matrigel Matrix were purchased from Fisher Scientific (Pittsburgh, PA). Transwell invasion assays were performed according to the manufacturer's protocol. Briefly, 2-3×10⁵ cells were seeded into each transwell, filled with serum-free culture medium. The bottom wells were filled with cell culture medium or Waymouth's medium with 10% FBS and either PEITC or Mito-PEITC. Controls received an equivalent amount of DMSO. After 36 h, cells were fixed with 10% formalin and stained with 5% crystal violet in 70% ethanol. Invaded cells were counted at a magnification of 10× in three randomly selected areas of each transwell, and the results were normalized to the control.

### Toxicity Studies of Mito-PEITC

All procedures were following the Houston Methodist Research Institute Animal Care and Use Committee. An 8-week subchronic toxicity study of Mito-PEITC was conducted in A/J mice (6 weeks old, from The Jackson Laboratory). During treatment, a Modified Irwin Screen employing 35 distinct measurements was used to assess sensorimotor, neurological, and autonomic nervous system function. A/J mice were treated with vehicle control or different doses of Mito-PEITC, given via oral gavage 5 days per week for 8 weeks. Body weights were measured weekly. After 8 weeks of treatment, serum was collected to measure alanine transaminase (ALT) and aspartate aminotransferase (AST) using an Ortho Clinical Diagnostic Vitros Fusion 5.1 analyzer. Mice were euthanized by CO₂ asphyxiation.

### In Vivo Lung Cancer Orthotopic and Brain Metastasis Models

An orthotopic model of lung adenocarcinoma cells (H2030-BrM3 cells) in athymic nude mice was used. Nude mice (5 weeks old) were anesthetized with isoflurane and placed in the right lateral decubitus position. A total of 1×10⁶ H2030-BrM3 cells in 50 g of growth factor reduced Matrigel in 50 μL of RPMI1640 medium were injected into the left lung through the left rib cage. One week after injection, mice were treated 5 days per week for 3 consecutive weeks with PEITC, Mito-PEITC or vehicle control. Tumor growth and metastases were monitored over time by bioluminescence (after injection of D-luciferin, 150 μg/g) using Lumina IVIS 100 imager (Perkin Elmer, Waltham, MA). Mice were euthanized on day 28.

For the brain metastasis studies, female NOD/SCID mice (age 5 weeks) were used. H2030-BrM3 cells (2×10⁵) were suspended in PBS (0.1 mL) and injected into the left ventricle under ultrasound guidance (ECHO 707, GE, Milwaukee, WI). One day after engrafting lung cancer cells in the arterial circulation, mice were randomly placed into treatment groups: vehicle control, PEITC, or Mito-PEITC. Mice were treated by oral gavage 5 days per week and metastases were monitored periodically by bioluminescence. Mice were euthanized at day 28 after the arterial injection of tumor cells.

### In Vivo Lung Cancer VC-Induced Tumorigenesis Study

To characterize the efficacy of Mito-PEITC on suppressing lung carcinogenesis, the VC-induced lung tumor model in A/J mice was used. Six-week-old female A/J mice were injected with VC (single i.p. dose, 16 mg/kg). One week after the VC injection, mice were randomized into the following control and treatment groups: (1) control, (2) 100 μmol/kg body weight PEITC, and (3) 4 μmol/kg body weight Mito-PEITC. Treatments were administered by oral gavage five times per week. After 18 weeks of treatment, the mice were euthanized. The three mice lung tumors from vehicle control and Mito-PEITC treatment group were collected and generated single-cell suspensions per the manufacturer's instructions. The rest of lungs were evaluated under a dissecting microscope to obtain the surface tumor count and individual tumor diameter. Tumor volume was calculated based on the formula V=4πr(3)/3. Tumor load (the total tumor volume in each mouse) was calculated from the sum of all tumors.

### In Vivo Lung Cancer Brain Metastasis Study

Six-week-old female SV129 mice were inoculated subcutaneously with LKR 13. Once the subcutaneous tumors reached a standard size (80 mm³), they were treated with intratumoral injections of Mito-PEITC three times. Seven days after tumor regression, 5×10⁵ LKR13-luc tumor cells in 100 μL of PBS were injected into the left ventricle of the cured mice and their age-matched littermates under ultrasound guidance (Vevo 3100, FUJIFILM Visual Sonics). Brain metastases were monitored periodically by bioluminescence using a Xenogen IVIS-200 system (Alameda, CA). The survival rate was monitored daily. After animals were euthanized, metastases were confirmed with ex vivo luminescence and histopathology.

### Depletion of CD4 and CD8 T Cells

Anti-CD4 (GK1.5 clone-rat IgG2b, 250 μg, BioXcell, BP0003-1) or anti-CD8 monoclonal antibodies (2.43 clone-rat IgG2b, 250 μg, BioXcell, BP0061) were injected i.p. one day before and one day after tumor inoculation, followed by repeat injections once per week. Eight days after the first i.p. injection, the spleens of the mice were collected to verify the depletion of CD4⁺ and CD8⁺ T cells using flow cytometry. The results showed greater than 99% depletion of each cell subset. Mice were implanted with LKR13 mouse lung adenocarcinoma cells in the body, and Mito-PEITC was administered by oral gavage.

### Combination of Mito-PEITC Treatment and Anti-PD-1 Treatment

Six-week female BA/6 mice were purchased from The Jackson Laboratory and inoculated with tumor cells as in 4.13 above. Animals were randomized into different treatment groups: (a) vehicle control, (b) anti-PD1 (Bioxcell, BE0146) (200 μg/mouse, every other day) (c) Mito-PEITC (4 μmol/kg), (d) combination. Mice started treatments on day 7 after tumor inoculation and tumor sizes were measured every two days. Mice were followed until death or euthanized earlier if tumors reached 2000 mm³.

### Flow Cytometry

Tumors were minced into 2 mm³ pieces and digested with mouse tumor dissociation buffer (Miltenyi Biotec, CA, 130-096-730) at 37° C. for 30 min and passed through a 40-μm nylon mesh to generate single-cell suspensions per the manufacturer's instructions. Red blood cells were removed by red blood cell lysis buffer (1.55 mM NH₄Cl, 1 mM KHCO₃, 0.1 mM EDTA). Isolated cells were first stained for viability and cell surface markers. Violet fluorescent reactive dye (Invitrogen, MP34955) was used to identify viable cells. Antibodies for staining surface markers included: BV786 anti-CD45 (Clone: 30-F11), PE anti-CD3 (Clone: 17A2), FITC anti-CD4 (Clone: GK1.5), BUV396 anti-CD8a (Clone: 53-6.7), FITC anti-CD11b (Clone: M1/70), APC/Fire750 anti-F4/80 (Clone: BM8), BUV396 anti-Ly6G (Clone: 1A8), PE/Cy7 anti-Ly6C (Clone: HK1.4), and APC/Fire750 anti-CD25 (Clone: PC61). For transcription factor staining, cells were first stained with surface markers, then fixed with fixation buffer (Biolegend, 420801), permeabilized with FoxP3/Transcription Factor Staining Buffer Set (eBioscience, 00-5523-00), and stained with APC anti-FoxP3 (Clone: FJK-16s). For intracellular cytokine staining, cells were stimulated for 4 h at 37° C. in Roswell Park Memorial Institute medium containing 10% fetal bovine serum, 2×mM 1-glutamine, 50 μM 2-mercaptoethanol, 1% penicillin-streptomycin, 0.2% cell stimulation cocktail (eBioscience, 00-4970-93), 0.1% monensin (eBioscience, 00-4505-51), and Brefeldin A (eBioscience, 00-4506-51). Cells were surface-stained with antibodies, fixed, and permeabilized using FoxP3/transcription factor staining buffer set, stained with intracellular cytokine staining buffer containing PE anti-IFN-γ and PE-Cy7 anti-TNF-α antibodies, and finally, analyzed by flow cytometry. Cells incubated in medium lacking PMA/ionomycin served as nonstimulated controls. To analyze myeloid-derived cells, cells were additionally incubated with anti-Mo CD16/CD32 (Invitrogen, 14-0161-82). These flow cytometry antibodies were purchased from either Biolegend, eBioscience, or BD Biosciences. Cells were analyzed using an LSR Fortessa X-20 flow cytometer (Becton Dickinson). Data were analyzed using FlowJo software (Treestar, Inc.).

scRNA-seq Analysis of Mouse Lung Tumors

For scRNA-seq, vinyl carbamate-induced lung tumors from the second experiment were harvested and pooled from different treatment groups at the end of the study, then minced and digested at 37° C. for 20 min with mouse tumor dissociation buffer (Miltenyi Biotec, Auburn, CA, USA) to generate single-cell suspensions per the manufacturer's instructions. The lung tumors were separated from the adjacent normal tissue before being pooled, and about five tumors were pooled from each mouse for scRNA-seq. CD45 is a transmembrane protein tyrosine phosphatase located on most nucleated hematopoietic cells; CD45 was used to differentiate immune cells from other non-immune epithelial and stromal cells. Single-cell suspensions were stained with CD45 surface markers, and the singular, CD45− cells were flow-sorted and centrifuged at 300×g for 5 min. Cells were then counted manually with a Neubauer chamber. Approximately 1.6×10 cells were loaded onto the 10× Chromium Controller per the manufacturer's instructions. scRNA-seq libraries were generated by Chromium Single Cell 3′ v3 Reagent Kits (10× Genomics, Pleasanton, CA, USA) and sequenced using NextSeq 500/550 High Output sequencing reagent Kits v2 (150 cycles) (Illumina) according to the manufacturer's protocol. There were two replicates for each of the experimental groups (control, Mito-HNK, Mito-LND, combination).

scRNA-seq Data Analysis

Raw sequencing data were de-multiplexed and converted to gene-barcode matrices using the Cell Ranger (version 2.2.0) mkfastq and count functions, respectively (10× Genomics). The mouse reference genome mm10 was used for alignment. Data were further analyzed in R (version 3.4.0) using Seurat (version 3). The number of genes detected per cell, the number of unique molecular identifiers (UMIs), and the percentage of mitochondrial genes were plotted. Outliers (i.e., cells that expressed less than 200 or more than 2500 genes) were removed to filter out doublets (two single cells) and dead cells. Differences in the number of UMIs and percentage of mitochondrial reads were regressed out. Raw UMI counts were normalized, and log transformed. The single-cell data were aligned and projected in a two-dimensional space through t-distributed stochastic neighbor embedding or uniform manifold approximation and projection to allow identification of different cell populations using the Seurat program. For the metabolic pathway analyses, the signature gene sets were downloaded from the KEGG database (http://www.kegg.jp, accessed on 1 Mar. 2022). When scoring cells for the expression of downloaded gene signatures, the AddModuleScore function was implemented in Seurat. The lung tumor cells were separated from normal cells using scCancer software version 2.2.1. Different cell types of the cells subjected to scRNA-seq were identified by combining the canonical marker analyses with the analyses from the SingleR software.

### Statistical Analysis

GraphPad Prism software was used for evaluating statistical differences between treatments. Student's t-test was applied for pairwise comparisons. Multiple comparisons (e.g., inhibition of viability data) were assessed using ANOVA with Tukey's post-hoc test. p-Values of <0.05 were considered significant.

### Results

### Synthesis and Toxicity of Mitochondria-Targeted (Mito)-PEITC

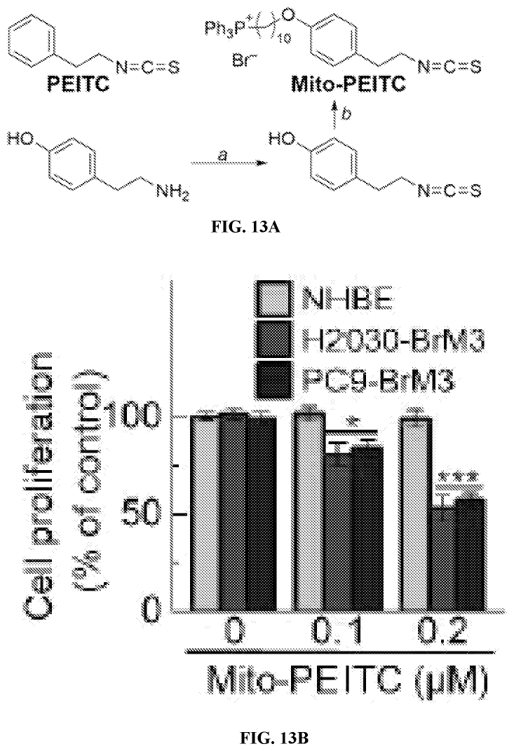

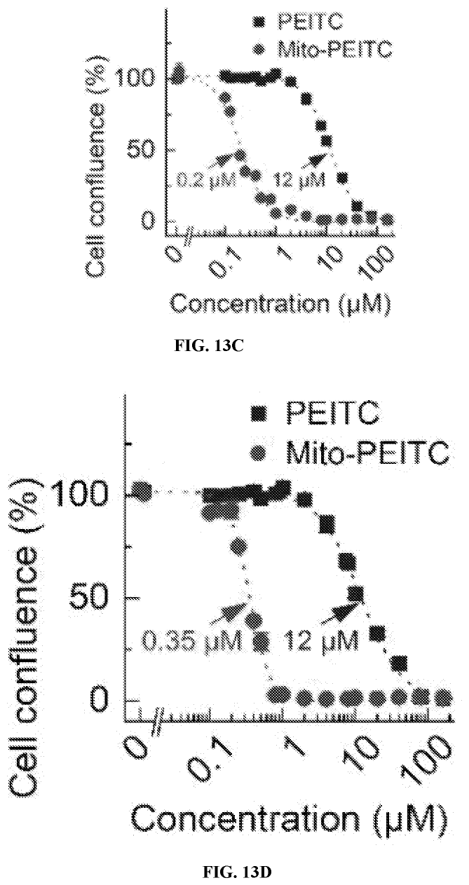

Mito-PEITC-C₁₀ (Mito-PEITC) was synthesized in two steps, starting from tyramine (1), generating PEITC with one TPP⁺ (triphenylphosphonium cation) via nucleophilic substitution between 4′-hydroxyphenethyl isothiocyanate (2) and a bromoalkyl-TPP⁺ reagent (FIG. 13A). The products were isolated by gel chromatography and purified by preparative HPLC. Their structures were characterized by NMR and mass spectrometry.

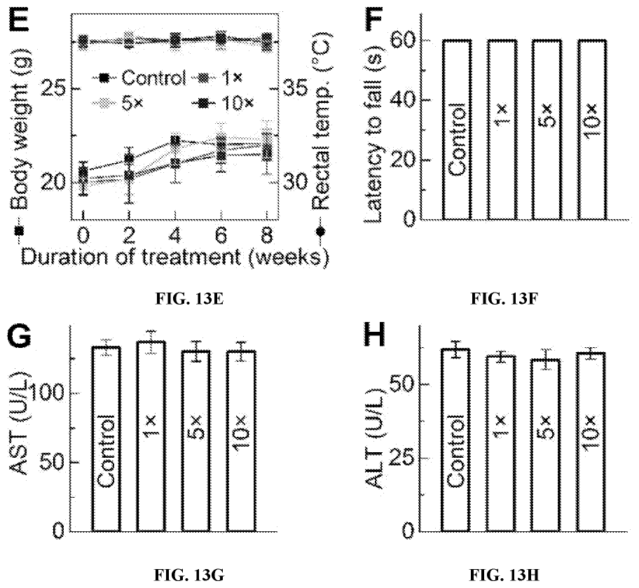

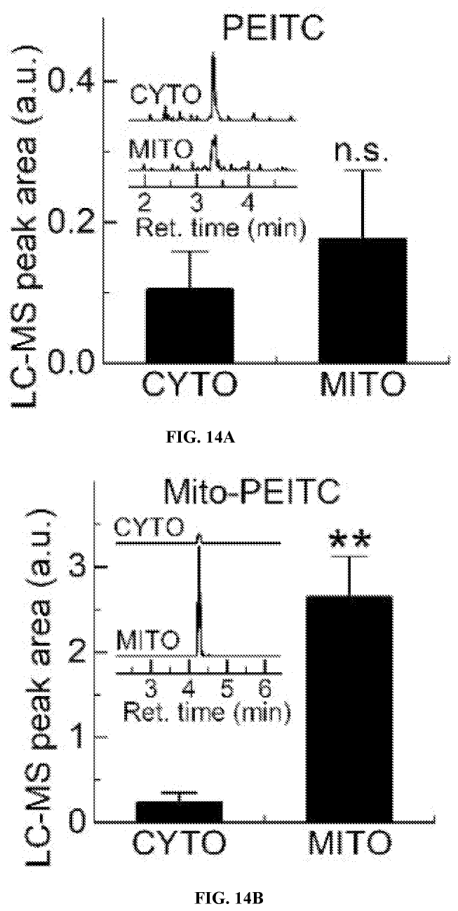

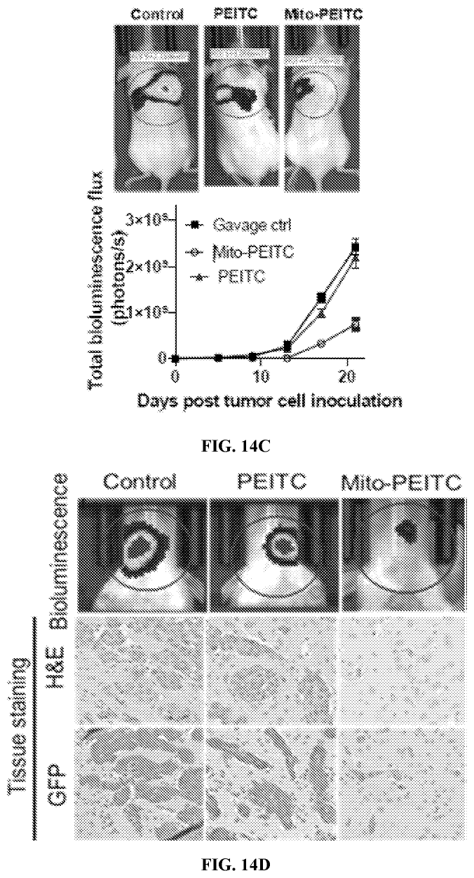

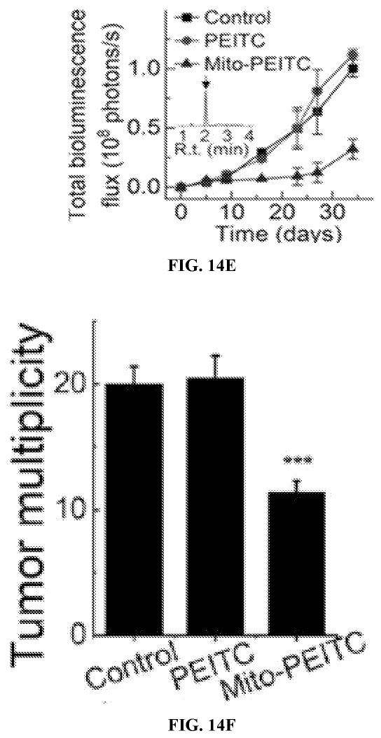

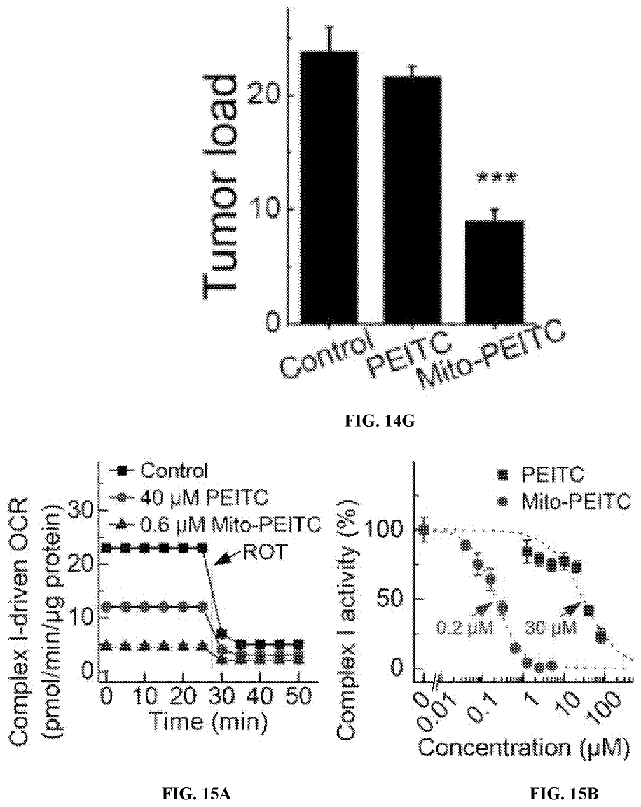

To compare the antiproliferative effects of PEITC and Mito-PEITC, human cancer cells were treated with PEITC or Mito-PEITC. Using the IncuCyte™ Live-Cell Imaging Analyzer, which provides real-time cell confluence data, it was found that Mito-PEITC consistently inhibits proliferation at significantly lower levels (IC₅₀=0.2-0.35 μM) than PEITC (IC₅₀=12 μM) in both lung adenocarcinoma (LUAD) cell lines tested (FIG. 13B-13D). LC-MS/MS analysis of the mitochondrial (MITO) and cytosolic (CYTO) fractions from cells treated for 24 h with 100 nM PEITC or Mito-PEITC was conducted. This analysis revealed significantly higher mitochondrial accumulation of Mito-PEITC (FIG. 14B) than of PEITC (FIG. 14A).

### Toxicity Study of Mito-PEITC

An 8-week toxicology study was conducted to evaluate potential toxicities of Mito-PEITC, especially on neural and muscle cells, using a Modified Irwin Screen that was developed as a comprehensive observational battery to screen for central nervous system (CNS) effects resulting from agents of interest (Irwin, S., Psychopharmacologia, 1968. 13(3): p. 222-57). Modified versions of this test are used extensively in the pharmaceutical industry (Lindgren, S., et al., J Pharmacol Toxicol Methods, 2008. 58(2): p. 99-109) and in academic research to identify changes in neurological function as a result of agent intoxication, neurotoxicity, or genetic manipulation (Moser, V. C., Toxicol Pathol, 2011. 39(1): p. 36-45; Crawley, J. N., 2000, New York: Wiley-Liss). The screen used here employs 35 distinct measurements to assess sensorimotor, neurological, and autonomic nervous system function (Olsen, C. M., et al., PLoS One, 2010. 5(11): p. e15085). The effective dose (ED) of Mito-PEITC is 4 μmol/kg. No significant differences between control and Mito-PEITC treated A/J mice were observed at doses up to 10×ED on body weight and temperature (FIG. 13E) or any of the 35 metrics tested (detecting sensorimotor, neurological, motor, and autonomic nervous system dysfunction) over eight weeks of treatment (FIG. 13F). Also, no toxic effects were observed in the liver damage indicators, AST or ALT enzymes (FIG. 13G, 13H). Thus, Mito-PEITC did not elicit any toxicities, even at doses up to 10×ED over 8-weeks treatment period.

### Efficacy Study of Mito-PEITC in Brain Metastasis and Orthotopic Model

A study using an orthotopic model of lung adenocarcinoma in nude mice was conducted. H2030-BrM3 cells were injected into the lung. One week after injection, mice were treated with the same dose of PEITC, Mito-PEITC (4 μmol/kg), or vehicle (corn oil) by oral gavage 5 days/week for 3 weeks. Equimolar doses were administered to illustrate the markedly enhanced potency of Mito-PEITC relative to PEITC. PEITC was not effective, as expected, because the applied dose (4 μmol/kg) is below the levels typically used in xenograft studies (Zhang, Q., et al., Mol Carcinog, 2020. 59(6): p. 590-603). In contrast, even at this relatively low dose, Mito-PEITC-treated mice exhibited significantly decreased tumor progression (>70% inhibition of the BLI signal intensity; FIG. 14C), demonstrating Mito-PEITC's markedly enhanced potency against lung cancer progression.

Cancer that metastasizes to the brain is one of the most intractable clinical problems of LUAD and a major cause of LUAD mortality (Goldberg, S. B., et al., Cancer J, 2015. 21(5): p. 398-403). About 10% of patients have brain metastases at the time of diagnosis, and 40-50% develop brain metastases during the course of LUAD (Schuster, D. P., et al., Am J Respir Cell Mol Biol, 2004. 30(2): p. 129-38). Halting the dissemination and metastasis of tumor cells is critical to reducing LUAD mortality. To test if Mito-PEITC affects brain metastasis, similar experiments in vivo using H2030-BrM3 cells were carried out. As shown in FIG. 14D, Mito-PEITC shows a significantly enhanced efficacy against brain metastasis of LUAD cells.

Efficacy of Mito-PEITC on lung tumorigenesis in A/J mice PEITC has shown specific inhibitory effects on rodent tumor development induced by the nicotine-derived, tobacco-specific carcinogen 4-(methylnitrosamino)-1-(3-pyridyl)-1-butanone (NNK) when administered during the initiation stage; however, it is less effective when given post-carcinogen exposure (Hecht, S. S., J Nutr, 1999. 129(3): p. 768s-774s; Hecht, S. S., Drug Metab Rev, 2000. 32(3-4): p. 395-411; Hecht, S. S., et al., Nat Rev Cancer, 2009. 9(7): p. 476-88; Hecht, S. S., et al., Cancer Lett, 2000. 150(1): p. 49-56). Although PEITC is capable of inhibiting NNK-induced lung carcinogenesis, it is not effective against lung tumorigenesis induced by benzo(a)pyrene, or B(a)P (Hecht, S. S., J Nutr, 1999. 129(3): p. 768s-774s; Hecht, S. S., Drug Metab Rev, 2000. 32(3-4): p. 395-411; Hecht, S. S., et al., Nat Rev Cancer, 2009. 9(7): p. 476-88; Hecht, S. S., et al., Cancer Lett, 2000. 150(1): p. 49-56). The study showed that Mito-PEITC inhibited vinyl carbamate (VC)-induced lung tumor development in A/J mice in a post-initiation model, where Mito-PEITC (4 μmol/kg) and PEITC (100 μmol/kg) treatments were initiated one week after VC and given by oral gavage 5 days/week for 20 weeks (FIGS. 14E and 14F). By contrast, PEITC was ineffective at a 25-fold higher dose (100 μmol/kg) with identical administration. Thus, Mito-PEITC, unlike its parental compound PEITC, significantly reduces VC-induced lung tumors in A/J mice with a favorable safety profile.

### PEITC and Mito-PEITC Inhibit Mitochondrial Complexes I & III Activity

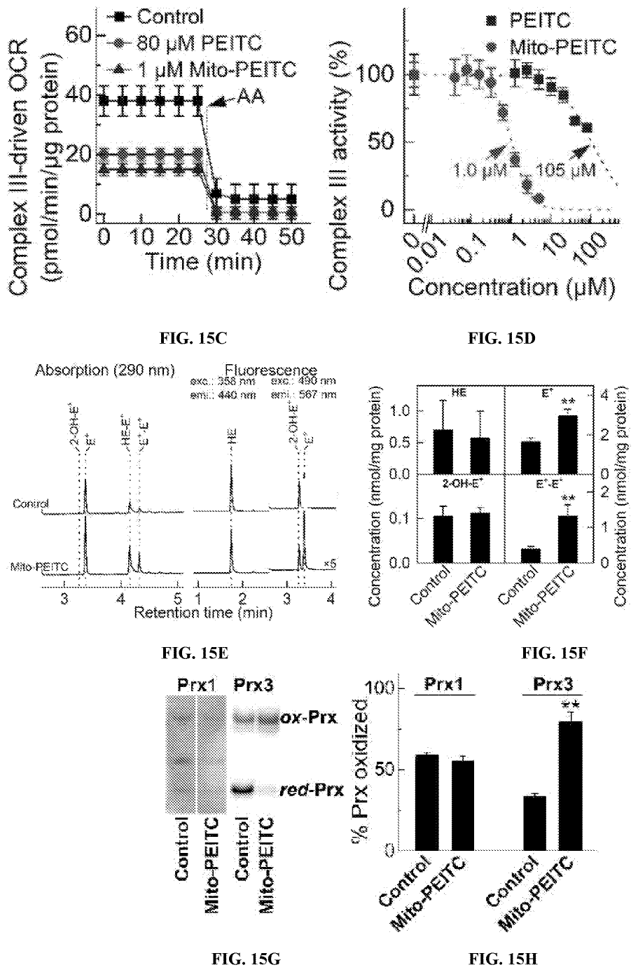

To test the effect of PEITC and Mito-PEITC on mitochondrial complex activity, H2030-BrM3 LUAD cells were pretreated for 24 h with the compounds, followed by cell permeabilization, the addition of complex substrates/inhibitors, and the measurement of oxygen consumption rate (OCR). Rotenone (complex I inhibitor) was used to confirm complex I activity, while antimycin A was used as an inhibitor of complex III (Salabei, J. K., et al., Nature Protocols, 2014. 9(2): p. 421-438). Both complexes I and III were inhibited by these compounds as expected. It was observed that Mito-PEITC was >100-fold more potent than PEITC in inhibiting both complexes I & III in H2030-BrM3 cells (FIG. 15A,15B). It was found that Mito-PEITC significantly increased ROS levels in LUAD cells, as determined by HPLC-based profiling of hydroethidine oxidation products (FIG. 15C). Unexpectedly, the superoxide-specific product 2-OH-E⁺ was not increased in Mito-PEITC (100 nM)-treated cells, but Mito-PEITC significantly increased cellular levels of ethidium (E⁺) and diethidium (E⁺-E⁺), indicating the formation of other, stronger oxidants (FIG. 15C).

### Mito-PEITC Induces Oxidation of Mitochondrial Peroxiredoxin-3

Prx proteins have been proposed to act not only as antioxidant enzymes but also as mediators of redox signaling (Bindoli, A., et al., 2008. 10(9): p. 1549-64; Forman, H. J., et al., Biochemistry, 2010. 49(5): p. 835-42; Forman, H. J., et al., J Mol Cell Cardiol, 2014. 73: p. 2-9). To identify possible direct targets of the increased ROS upon complex I & III inhibition and GSH depletion, H2030-BrM3 cells were treated with 100 nM Mito-PEITC for 24 hours and measured the relative amounts of oxidized and reduced cytosolic Prx1 and mitochondrial Prx3 by western blot. Mito-PEITC led to significant oxidation of mitochiondrial Prx3, but had no effect on the redox status of cytosolic Prx1 (FIG. 15D). This implies that Mito-PEITC-induced redox stress occurs primarily in mitochondria. Importantly, oxidized Prx can directly oxidize STAT3 to inhibit STAT3 activity (Sobotta, M. C., et al., Nat Chem Biol, 2015. 11(1): p. 64-70), which suggests a possible mechanism for Mito-PEITC action worthy of further investigation.

### Role of STAT3 in Mediating the Chemopreventive Effect of Mito-PEITC on LUAD Invasion

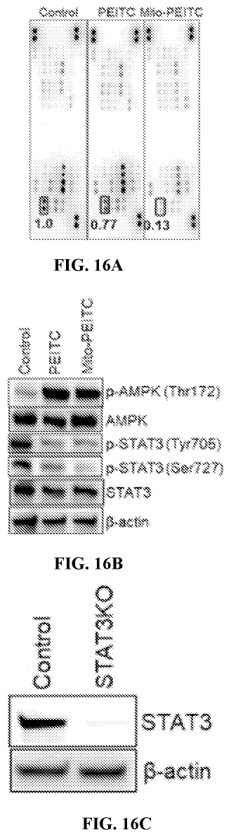

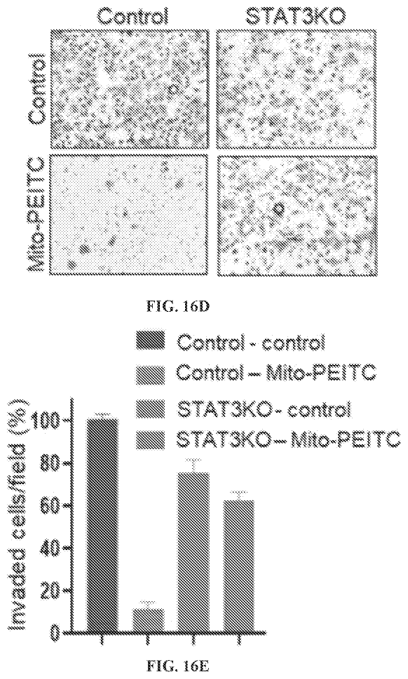

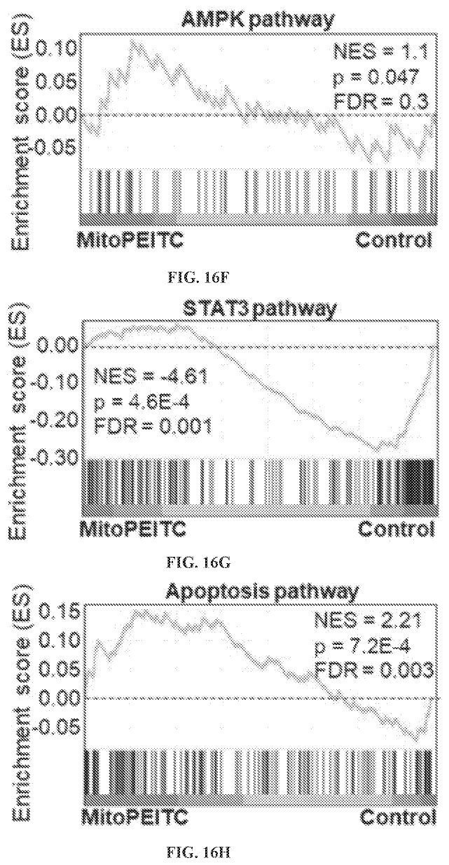

Based on the previous findings, the potential mechanisms by which Mito-PEITC inhibits LUAD cell brain metastasis with a receptor tyrosine kinase assay was examined (FIG. 16A), which has been used extensively to study the mechanisms of candidate agents (Vazquez-Martin, A., et al., Sci Rep, 2013. 3: p. 2560). Increased phosphorylation of AMPK and decreased phosphorylation of STAT3 was identified. Because AMPK and STAT3 can play important roles in regulating mitochondrial activity, apoptosis, proliferation, and migration (Feng, Y., et al., Cell Death & Disease, 2014. 5; Hwang, A. B., et al., Proc Natl Acad Sci USA, 2014. 111(42): p. E4458-67; Liu, H. T., et al., Blood, 2003. 102(1): p. 344-352; Zhang, Q., et al., J Biol Chem, 2013. 288(43): p. 31280-8), the effects of PEITC and Mito-PEITC on AMPK^{Thr172} and STAT3^{Ser727} phosphorylation were then examined by western blot (FIG. 16B). Mito-PEITC potently inhibits STAT3^{Ser727} and STAT3^{Tyr705} phosphorylation in H2030-BrM3 cells, while inducing AMPK^{Thr172} phosphorylation.

STAT3^{Ser727} phosphorylation is required for maximal STAT3 transcriptional activity (Wen, Z., Cell, 1995. 82(2): p. 241-50), which drives the invasiveness of tumor cells. The effect Mito-PEITC on invasiveness was studied using STAT3 knockout H2030-BrM3 cells generated using CRISPR/Cas9 (Vazquez-Martin, A., et al., Sci Rep, 2013. 3: p. 2560). The anti-invasive effects of Mito-PEITC (24 h) were found to be significantly abrogated in STAT3-deficient cells (FIGS. 16C, 16D and 16E). RNAseq analysis conducted on LKR-13 cells showed similar activation of the AMPK pathway and inhibition of the STAT3 pathway (FIG. 16F, 16G), accompanied by upregulation of the apoptosis pathway (FIG. 16H). This induction of apoptosis in H2030-BrM3 cells was confirmed by flow cytometry, using Mito-PEITC concentrations that were 100-fold lower (0.2 μM) than required for PEITC (20 μM) (FIG. 16I).

Mito-PEITC Reacts Directly with Reduced Glutathione

One of the major proposed cellular targets of PEITC is reduced form of mitochondrial glutathione (GSH), the depletion of which is thought to contribute to the anticancer effects of PEITC (Zhang, Y., et al., Biochem Biophys Res Commun, 1995. 206(2): p. 748-55; Kolm, R. H., et al., Biochem J, 1995. 311 (Pt 2)(Pt 2): p. 453-9; Meyer, D. J., et a., Biochem J, 1995. 306 (Pt 2)(Pt 2): p. 565-9; Chen, G., et al., Antioxid Redox Signal, 2011. 15(12): p. 2911-21).

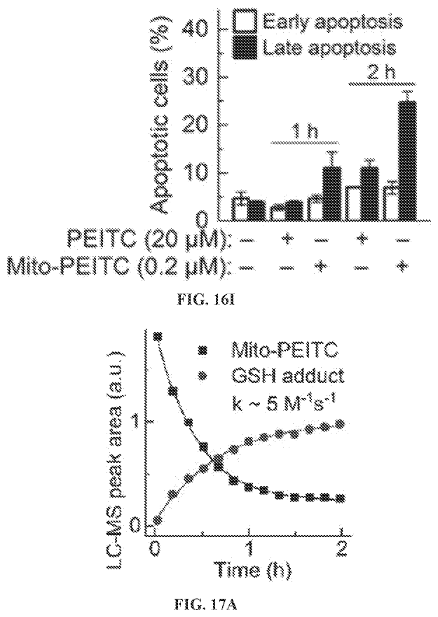

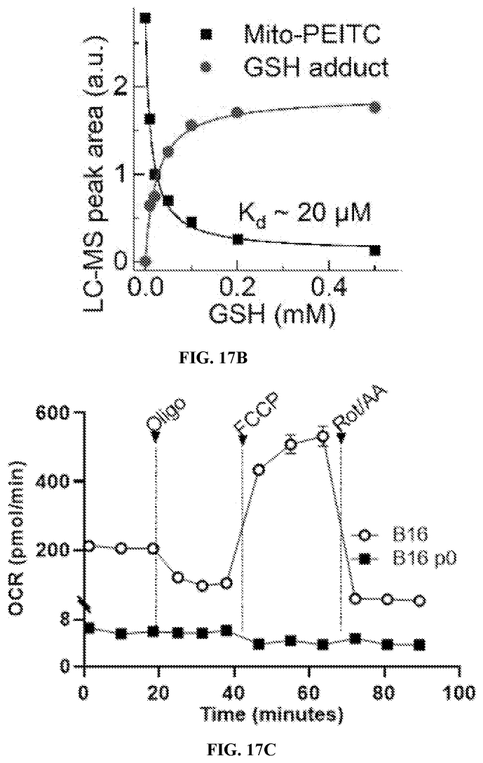

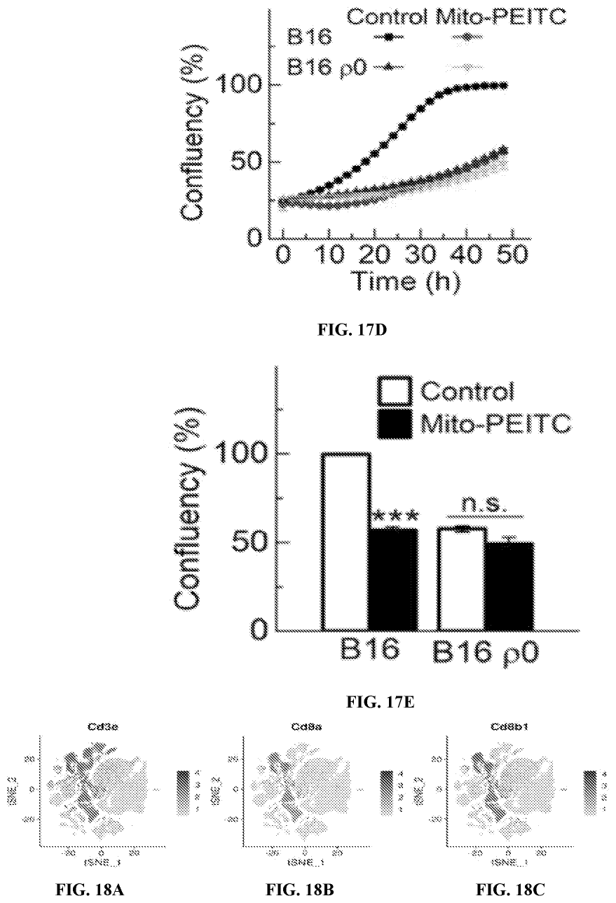

It was tested whether Mito-PEITC could also react with and deplete GSH. It was found that Mito-PEITC reacts directly with GSH with an estimated reaction rate constant of k=5 M⁻¹s⁻¹ at 25° C. (FIG. 17A), which is similar to the value reported previously for PEITC (Meyer, D. J., et a., Biochem J, 1995. 306 (Pt 2)(Pt 2): p. 565-9). It was also found that this process is reversible and estimated the dissociation constant K_{d}=20 μM of the adduct (FIG. 17B). GSH adducts of Mito-PEITC may thus act as a store of Mito-PEITC, effectively using GSH as a shuttle to deliver Mito-PEITC to mitochondrial protein target(s) via the process of transthiocarbamoylation (Shibata, T., et al., J Biol Chem, 2011. 286(49): p. 42150-42161).

To delineate the involvement of mitochondria in Mito-PEITC's anti-cancer effects, Mito-PEITC was tested in mtDNA-depleted B16 ρ0 cells and B16 cells (Dong, L.-F., et al., eLife, 2017. 6: p. e22187). The mitochondrial respiration assay confirmed a complete loss of mtDNA in ρ0 cells (FIG. 17C). Mito-PEITC treatment inhibited cell proliferation in parental cells by ~50% (FIG. 17D, 17E), but showed no significant effects on ρ0 cells, indicating that loss of mitochondrial function completely abrogates the anti-proliferative effects of Mito-PEITC.

scRNA-Seq Revealed Profound Immune Alterations in the Microenvironment of Mito-PEITC-Treated Tumors.

To better understand the effects of Mito-PEITC on immune function, scRNA-seq was performed on both tumor cells (CD45−) and immune cells (CD45+) isolated from VC-induced lung tumors in mice from the different treatment groups. The CD45+ immune cells consisted of CD8+ T cells, CD4+ T cells, B cells, natural killer (NK) cells, dendritic cells (NC), macrophages and neutrophils (FIG. 17A, 17B). With Mito-PEITC treatment, the percentage of CD8+ T cells increased while the percentage of macrophages decreased (FIG. 17B). The CD45− cells included tumor cells, epithelial cells, endothelial cells, and fibroblasts (FIG. 17C). A significant decrease of tumor cells in the lung tumor samples under Mito-PEITC treatment was observed (FIG. 17D).

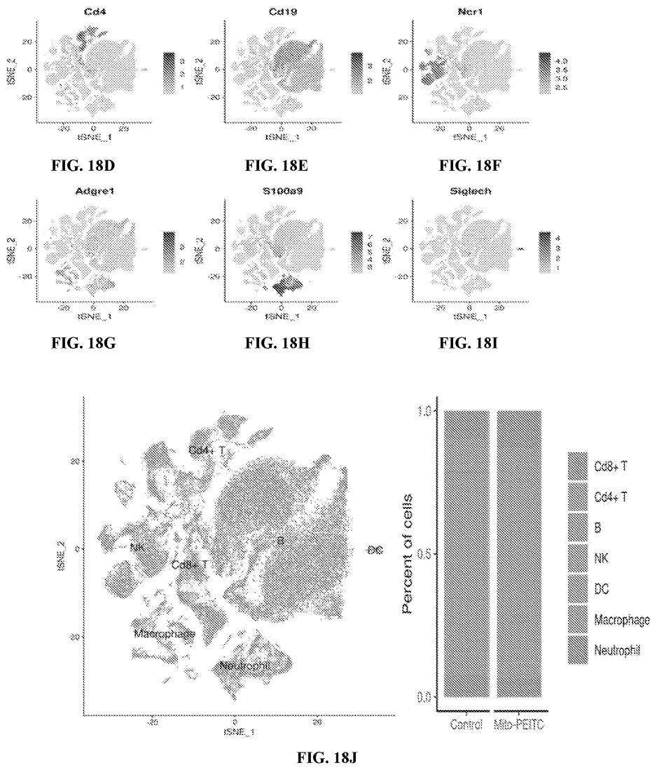

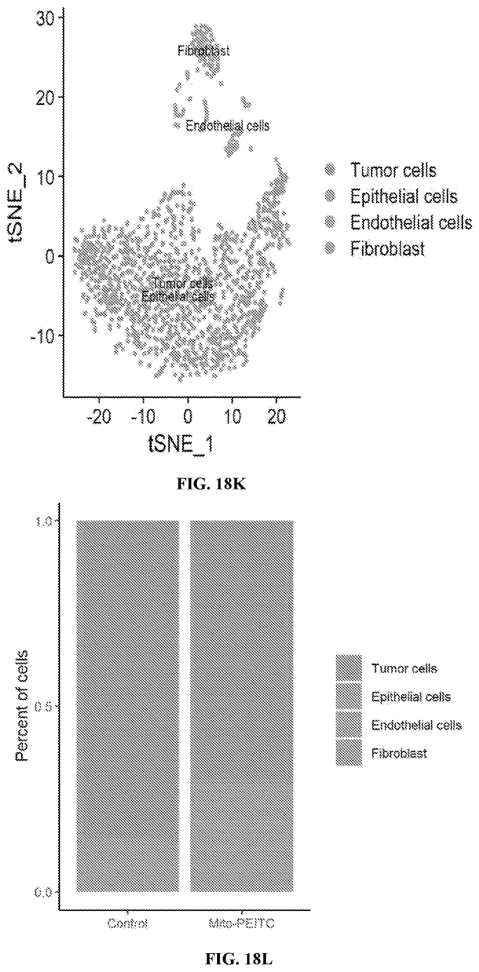

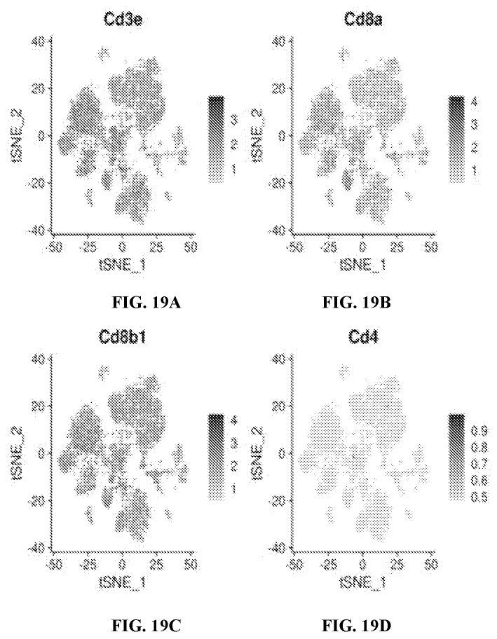

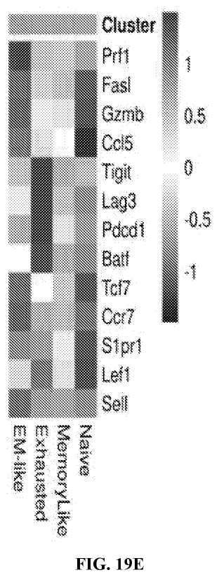

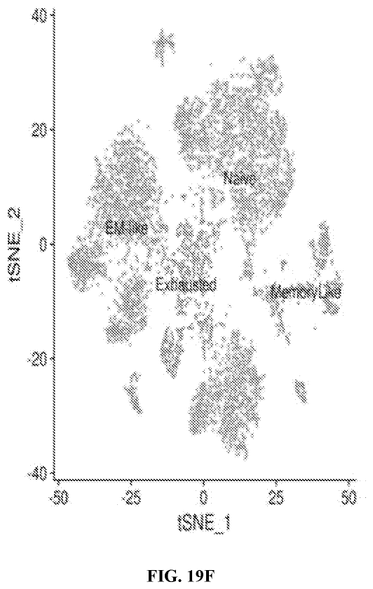

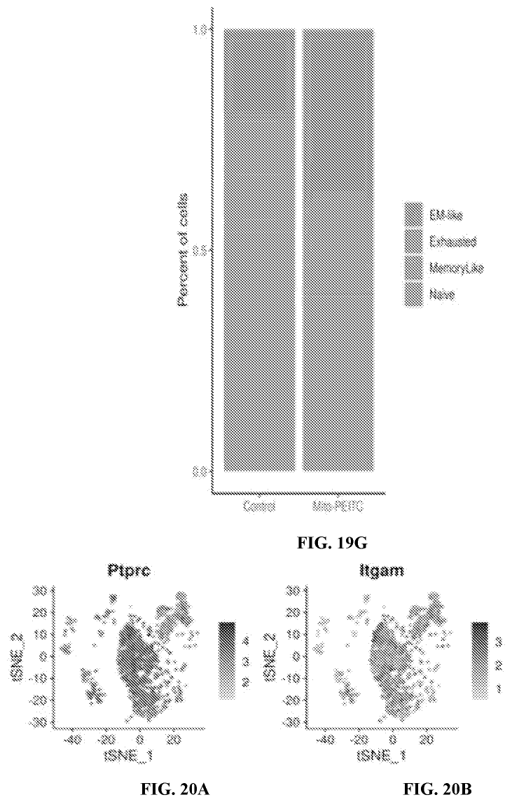

Next unsupervised clustering of CD8+ tumor-infiltrating lymphocytes (TILs) using the TILPRED program (https://github.com/carmonalab/TILPRED) was conducted. The presence of four CD8+ TIL subsets with distinct transcriptomic profiles were identified (FIG. 18A-18C). The CD8+ subsets included naïve, effector-memory (EM)-like, memory-like, and exhausted T cells. Mito-PEITC treatment significantly altered the percentages of distinct CD8+ TIL subsets in the TME (FIG. 18D). The proportion of CD8+ T cells mediating antitumor function (i.e., EM-like CD8+ TILs) was increased significantly by Mito-PEITC treatment compared with control. These data suggest that Mito-PEITC improves the overall composition of the antitumor CD8+ TILs.

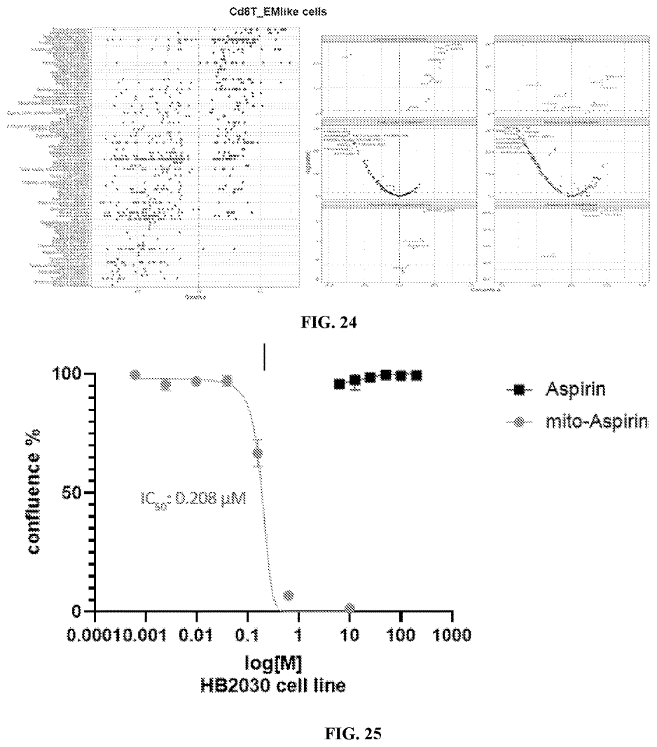

Metabolism pathway changes in the tumor cells and CD8+ EM-like T cells that could be associated with Mito-PEITC treatment using COMPASS were further analyzed (Wagner, A., et al., Cell, 2021. 184(16): p. 4168-4185 e21). Almost all the major metabolism pathways in the lung tumor cells were significantly down-regulated by Mito-PEITC treatment (FIG. 23). In the CD8+ EM-like T cells, it was identified that the main energy metabolism pathways, including glycolysis metabolism, TCA cycle, OXPHOS and glutamine metabolism, were up-regulated by Mito-PEITC treatment (FIG. 24). The differences in how Mito-PEITC treatment affects the major metabolism pathways between the tumor cells and the anti-tumor CD8+, EM-like T cells favor the weakening of tumor cells but enhancement of the cytotoxic CD8+ T cells, which may contribute to the anti-tumor efficacy of Mito-PEITC treatment.

### Mito-PEITC Treatment Increased Tumor-Infiltrating T Cells and Reduced G-MDSCs and Tregs in Tumors

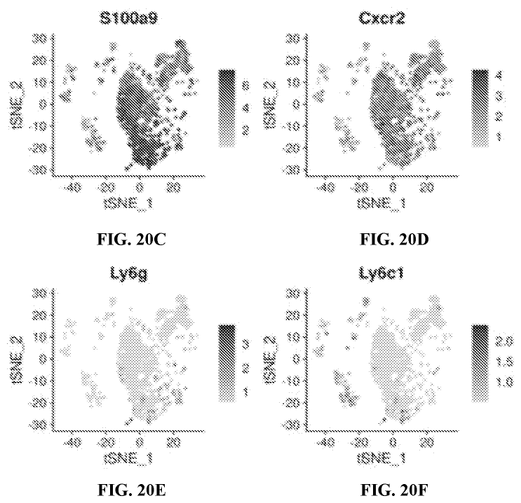

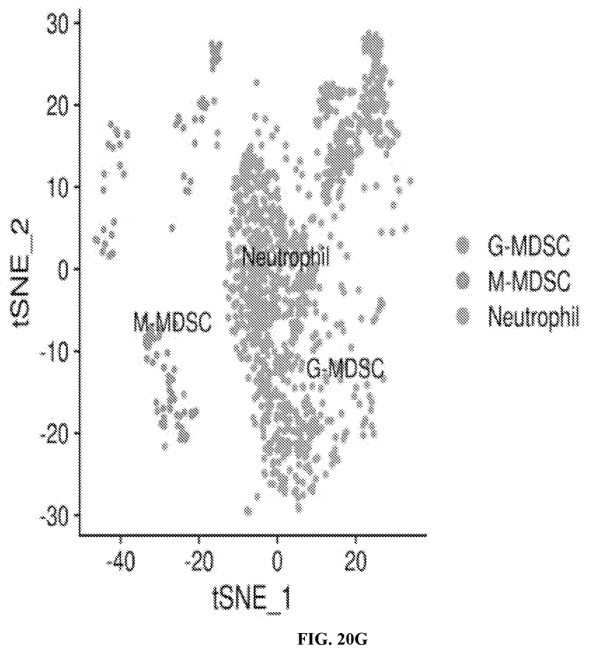

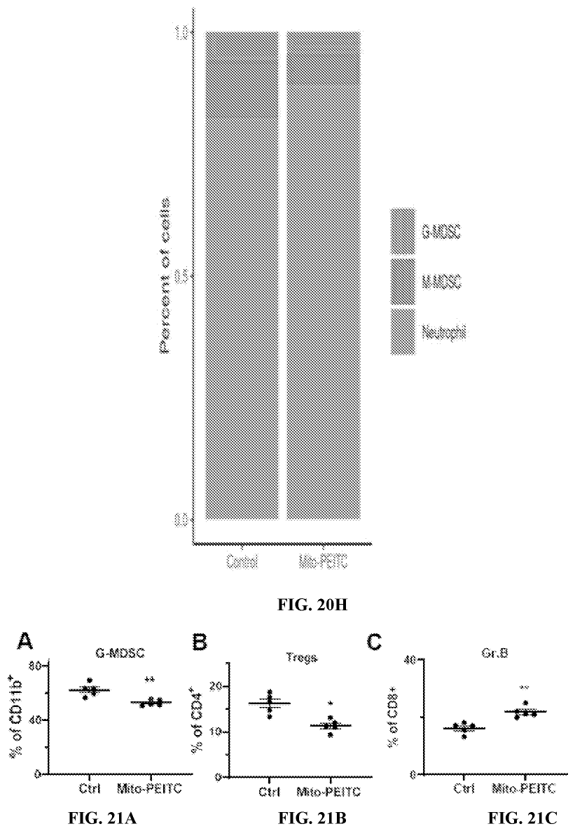

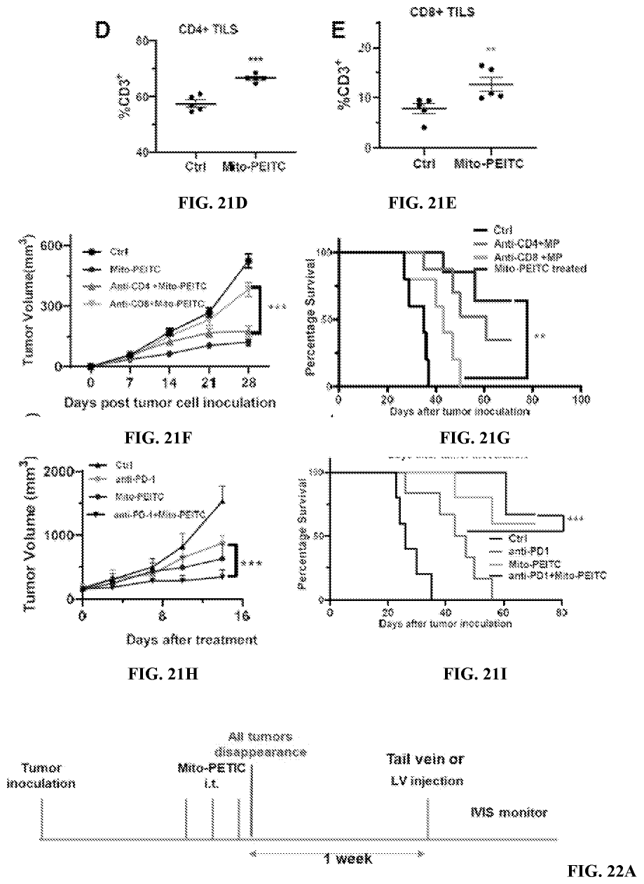

In addition to increasing the granzyme B-expressing cytotoxic CD8+, EM-like T cells, Mito-PEITC treatment also led to the reduction of immunosuppressive granulocyte-like myeloid-derived suppressor cells (G-MDSCs) and monocytic MDSCs (M-MDSCs) (FIG. 20A-20C). To validate these results from scRNA-seq, multicolor flow cytometric analyses were conducted from the TME of mice with implanted LKR13 cells that were treated with Mito-PEITC. A significant reduction of G-MDSCs andr Treg cells, as well as a significant increase of Gran B CD8+ T cells, within tumors from the Mito-PEITC-treated mice were observed (FIG. 21A-21C). Significant increases in tumor-infiltrating CD4+ and CD8+ T cells in tumors from mice treated with Mito-PEITC was also observed FIG. 21D, 21E). The inhibition of G-MDSCs and promotion of cytotoxic CD8+ T cells observed in the flow cytometry experiments matched the scRNA-seq results (FIG. 20A-20C).

To determine the roles of different immune cells during treatment with Mito-PEITC, either CD4+ or CD8+ T cells were depleted by intraperitoneal injection of anti-mouse CD4 monoclonal antibody (mAb) or anti-mouse CD8 mAb in SV129 mice before tumor inoculation and during neoadjuvant treatment. These mice were implanted with LKR13 mouse lung adenocarcinoma cells in the body, and Mito-PEITC was administered by oral gavage. Specific T cell depletion was verified by flow cytometry. While treatment with Mito-PEITC caused a regression of the established LKR13 tumors in mice in which T cells had not been depleted, the antitumor effect of Mito-PEITC was abolished in mice in which CD8+ T cells were depleted. Mito-PEITC was still effective in mice that were depleted of CD4+ T cells (FIG. 21F), suggesting the antitumor effects of Mito-PEITC appear to require the presence of CD8+ T cells (and to a lesser extent, the CD4+ T cells). Using T cell-depleting antibodies, it was that the efficacy of glutamine antagonist therapy was mainly dependent upon the activity of the CD8+ T cell subset of the adaptive immune response.

Elevated Tregs and G-MDSCs have been demonstrated to be associated with poor outcomes of anti-PD1 treatment. Mito-PEITC was combined with PD-1 checkpoint immunotherapy. It was observed that Mito-PEITC plus anti-PD-1 antibody treatment showed striking antitumor efficacy. Furthermore, a notable survival advantage was seen in mice given Mito-PEITC plus anti-PD-1 antibodies compared with the control groups (FIG. 21G).

Local Injection of Mito-PEITC into a Primary Tumor Site Prevents Lung and Brain Metastasis.

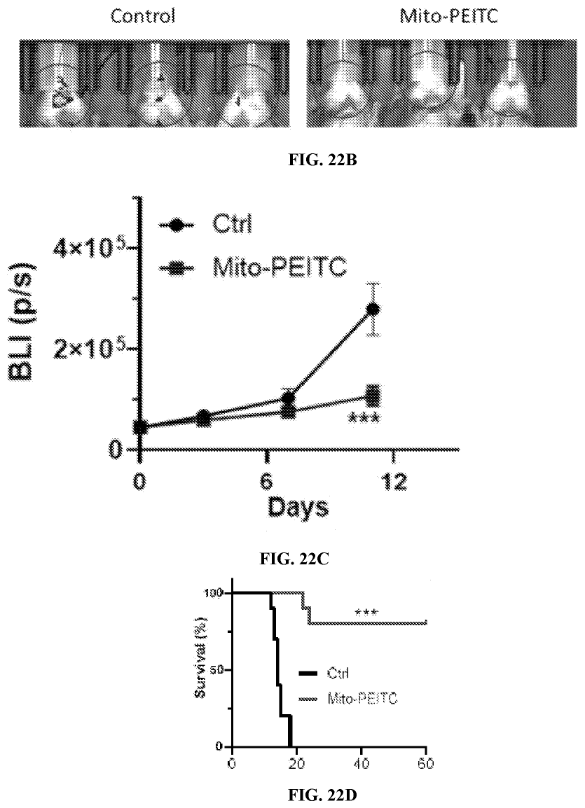

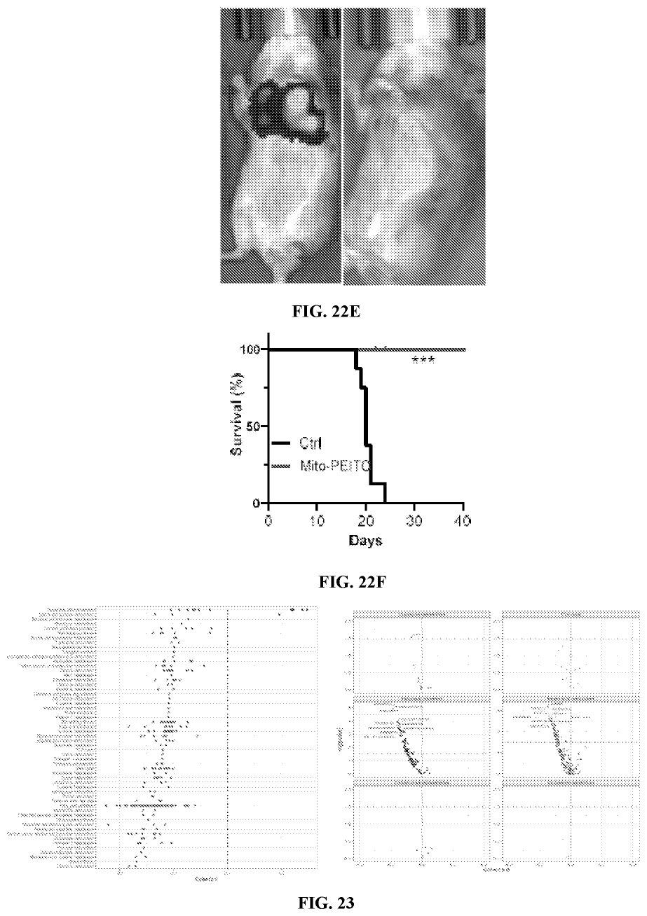

To test whether local injection into a primary tumor can prevent lung and brain metastasis, mice were implanted with LKR13 mouse lung adenocarcinoma cells as illustrated in FIG. 22A. LKR13 cells were inoculated subcutaneously into SV129 mice. Once the subcutaneous tumors reached a standard size (80 mm³), they were treated with intratumoral injections of Mito-PEITC three times. Mito-PEITC caused complete tumor regression at the local injection site. One week after the last injection, it was tested if the induction of Mito-PEITC for an antitumor immune response was sufficient to reject a tumor rechallenge. Cured mice were injected with LKR13 cells by left ventricle injection for brain metastasis or by tail vein injection for lung metastasis. For brain metastasis, naïve control mice died before day 20. In contrast, 8 out of 10 cured mice demonstrated a longer survival advantage (FIG. 22B-22D). The lung metastasis results also showed the survival advantage, all mice survived after 40 days of rechallenge, while all naïve control mice died within 25 days (FIG. 22E-22F). These results suggest that the initial in situ Mito-PEITC may have elicited antitumor immune changes that are durable over time and could prevent or treat cancers and their distant metastases.

### Discussion

NSCLCs are the most common lung cancers, and approximately 40% of NSCLCs are LUADs (Siegel, R. L., et al., Cancer statistics, 2022. CA: A Cancer Journal for Clinicians, 2022. 72(1): p. 7-33; Tian, S., Oncol Lett, 2017. 14(5): p. 5464-5470; Anusewicz, D., et al., Scientific Reports, 2020. 10(1): p. 21128). Current and former smokers have increased LUAD risk (Liu, B., et al., Anticancer Agents Med Chem, 2022. 22(8): p. 1541-1550). Brain metastases are one of the most intractable clinical problems associated with LUAD and one of the leading causes of LUAD mortality (Goldberg, S. B., et al., Cancer J, 2015. 21(5): p. 398-403; Sperduto, P. W., et al., JAMA Oncol, 2017. 3(6): p. 827-831). Thus, discovering small molecule agents that can prevent primary and metastatic LUAD is a critical unmet need.

PEITC has shown potential as one such small molecular agent. In previous studies, PEITC had demonstrated some promise in preventing carcinogenesis, but this effect was observed only in certain models and only if administered prior to tumor initiation (Hecht, S. S., J Nutr, 1999. 129(3): p. 768s-774s; Hecht, S. S., Drug Metab Rev, 2000. 32(3-4): p. 395-411; Hecht, S. S., et al., Nat Rev Cancer, 2009. 9(7): p. 476-88). These limits make PEITC alone insufficient to prevent lung cancer in smokers and those with precancerous lesions.

TPP⁺-based targeting of molecules has several advantages: First, the TPP⁺ cation improved stability. Second, it has low chemical reactivity toward cellular components. Finally, TTP⁺ allows for the ability to modify hydrophobicity by tethering alkyl linker side chains to various drugs. In this study, a compound was designed, Mito-PEITC, by conjugating PEITC with TPP⁺ via an alkyl linker to promote mitochondrial accumulation of PEITC. By doing so, mitochondrial accumulation was markedly enhanced and the ability to inhibit LUAD progression and metastasis. Mito-PEITC potently inhibits mitochondrial complexes I & III, induces ROS generation, Prx oxidation, AMPK activation, and inhibits mitochondrial p-STAT3^{ser727}. Most excitingly, Mito-PEITC can do all these things at significantly lower concentrations than PEITC. Results demonstrate that PEITC's regulation of mitochondrial complexes I & III serves as a key mechanism for its action as a cancer chemopreventive agent.

In the 8-week in vivo toxicity study, it was showed that Mito-PEITC displayed no systemic toxicities, such as body weight loss or elevated liver enzymes. The nervous system function was also tested and found their significant differences between control and Mito-PEITC treated mice at doses up to 10×ED. Mito-PEITC (at 4 mol/kg) significantly inhibited lung cancer metastases from growing in the brain, whereas PEITC does not inhibit brain metastasis even at a 25-fold higher dose. Mito-PEITC also inhibited lung tumor growth in orthotopic models. In the VC-induced primary tumor model, Mito-PEITC exhibited significantly stronger efficacy than PEITC. These results suggest that Mito-PEITC can be effective for treating lung cancer and its metastases.

While the inhibition of complex I and III and the resulting ROS generation are early events that occur in cancer cells exposed to Mito-PEITC, other subsequent events may ultimately contribute to its antiproliferative effects. AMPK is a master regulator of cellular energy homeostasis and is typically activated in response to nutrient or energy deprivation. Constitutive activation of STAT3 is also important for growth and progression in many tumors. The data show that the anti-proliferative and anti-invasive effects of Mito-PEITC involve the inhibition of mitochondrial respiration, downstream STAT3 activation, and increased AMPK activation (phosphorylation). Specifically, Mito-PEITC inhibits complex I and complex III activities in cancer cells, induces redox stress, promotes oxidized Prx3, and inhibits mitochondrial STAT3 phosphorylation (p-STAT3⁷²⁷) in LUAD cells. Mito-PEITC decreased the phosphorylation of AMPK by ~50% in lung cancer cells. The RNA-seq analyses confirmed the similar activation of the AMPK pathway and inhibition of the STAT3 pathway. Peroxiredoxin oxidation is another potential link between increased ROS and the inhibition of STAT3. Mito-PEITC significantly increased the oxidation of mitochondrial Prx3, but didn't show an effect on the redox status of cytosolic Prx3. Taken together, these data start to elucidate the mitochondrial mechanism of action of Mito-PEITC.

The study showed that Mito-PEITC significantly prevented metastases from growing in the brain and the lungs. Analyses of the potential effects of Mito-PEITC on immune cells in the TME showed that Mito-PEITC treatment significantly reduces both G-MDSCs and Tregs. Interestingly, Mito-PEITC can enhance the efficacy of the immune-checkpoint blockade with anti-PD-1 in mice. The data suggest that the enhanced antitumor efficacy of Mito-PEITC is a consequence of reduced G-MDSCs and Tregs within the TME and a concomitant increase in functional tumor-infiltrating CD8+ T cells. Given their prominent roles in tumor immune evasion, targeting G-MDSCs and Tregs with Mito-PEITC could be an attractive approach to modulate tumor immunity to prevent and treat cancers.

To summarize, the study demonstrates that Mito-PEITC inhibits mitochondrial complexes I and III, which leads to Prx oxidation, AMPK activation, and the inhibition of STAT3 in lung cancer cells. Mito-PEITC is a chemopreventive agent in mouse models of LUAD progression and brain metastasis. These findings provide key insights into the chemopreventive mechanisms and potential of Mito-PEITC as a safe and effective agent to improve the efficacy of PEITC in lung cancer treatment.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

This listing of claims replaces all prior versions and listings of claims in the application:

1. A compound of formula I 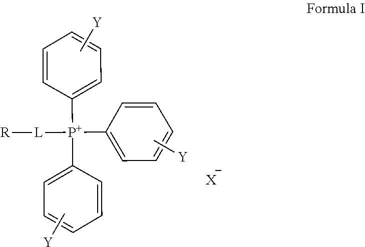
or pharmaceutically acceptable salts, prodrugs, or derivatives thereof,
wherein:
L is selected from an unsubstituted or substituted C1-C20 alkyl, unsubstituted or substituted C2-C20 alkenyl, unsubstituted or substituted C1-C20 alkynyl, unsubstituted or substituted aryl, unsubstituted or substituted cycloalkyl, or unsubstituted or substituted heterocycle;
Y are each independently selected from H, —OR^{a}, haloalkyl, or halogen;
R^{a} is an unsubstituted or substituted alkyl, or haloalkyl; and
X is an anion;
R is selected from: 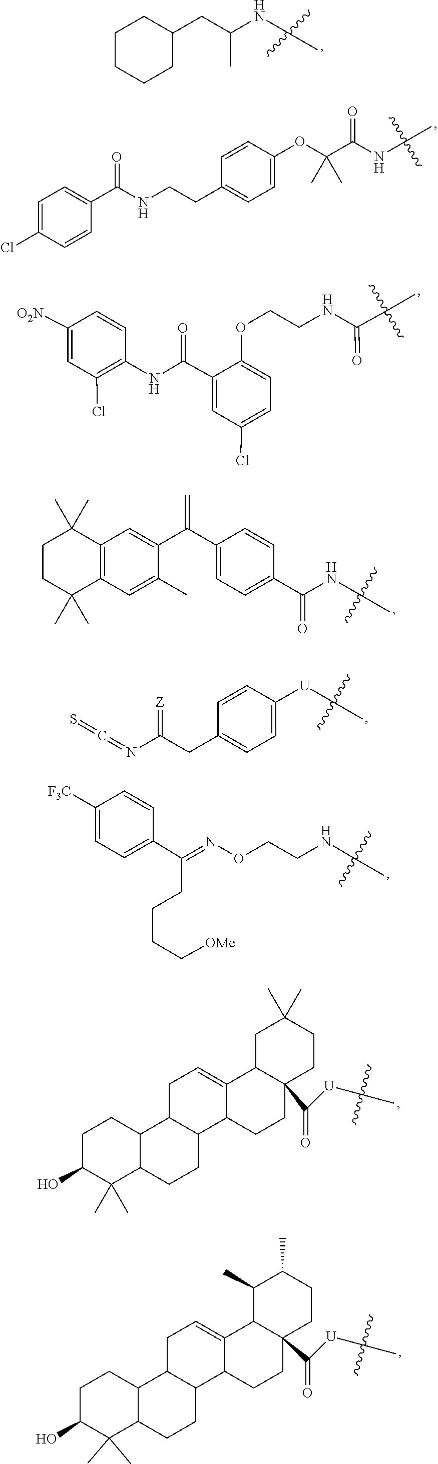 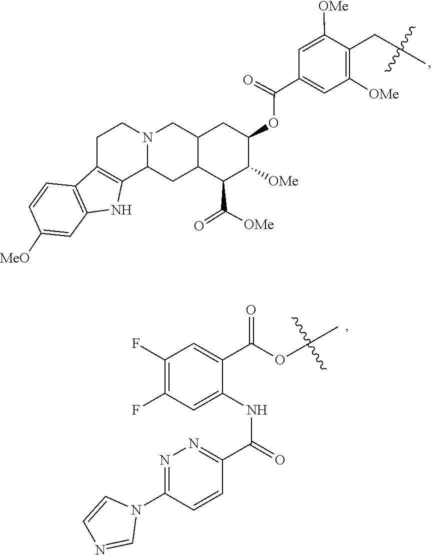 [SMILES: *Cc1c(OC)cc(C(=O)O[C@@H]2CC3CN4CCc5c([nH]c6cc(OC)ccc56)C4CC3[C@H](C(=O)OC)[C@H]2OC)cc1OC ; *OC(=O)c1cc(F)c(F)cc1NC(=O)c1ccc(-n2ccnc2)nn1] 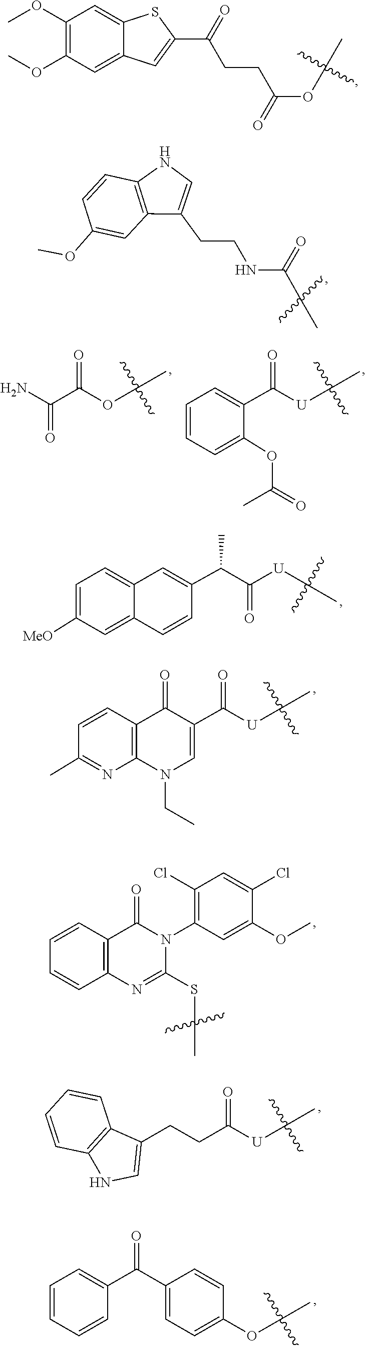 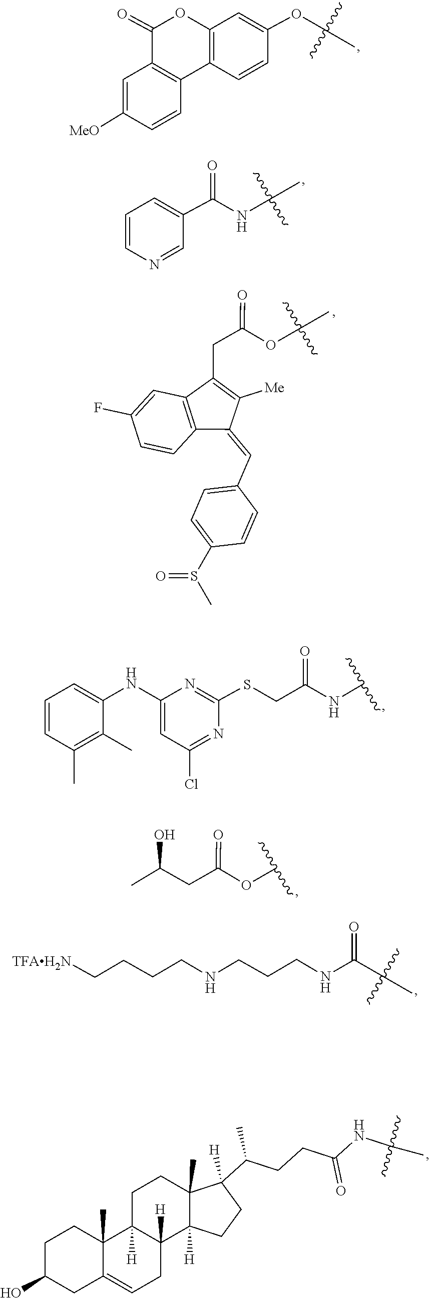 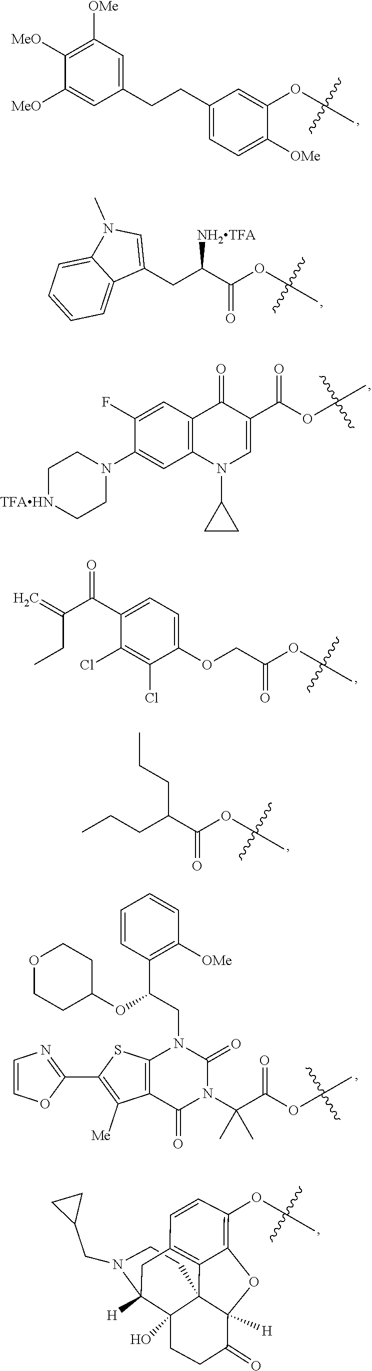 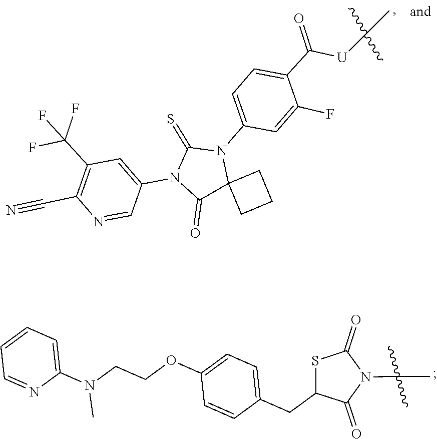
U is O, S, or NH; and
Z is —H₂ or —CH₂CH₂.

2. The compound of claim 1, wherein R is

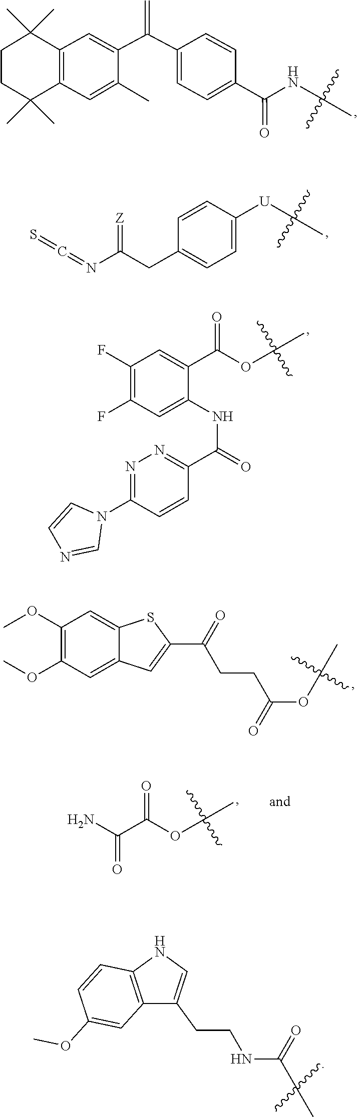

3. The compound of claim 1, wherein R is

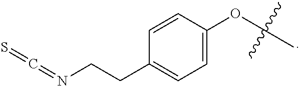 [SMILES: *Oc1ccc(CCN=C=S)cc1]

4. The compound of claim 1, wherein X is a halogen, trifluoroacetic acid, triflate, hexafluorophosphate or acetic acid.

5. The compound of claim 1, wherein L is phenyl.

6. (canceled)

7. (canceled)

8. The compound of claim 1, wherein L is poly(ethylene glycol) (PEG).

9. The compound of claim 1, wherein L is a substituted heterocycle.

10. (canceled)

11. The compound of claim 1, wherein L is unsubstituted C1-C20 alkyl.

12.-18. (canceled)

19. The compound of claim 1, wherein Y is located at an ortho, meta, or para position.

20-22. (canceled)

23. The compound of claim 1, wherein Y is located at an ortho and meta positions.

24. The compound of claim 1, wherein Y is located at an ortho and para positions.

25. The compound of claim 1, wherein Y is located at a meta and para positions.

26. The compound of claim 1, wherein Y is located at an ortho, meta, and para positions.

27-29. (canceled)

30. The compound of claim 1, wherein the compound is selected from:

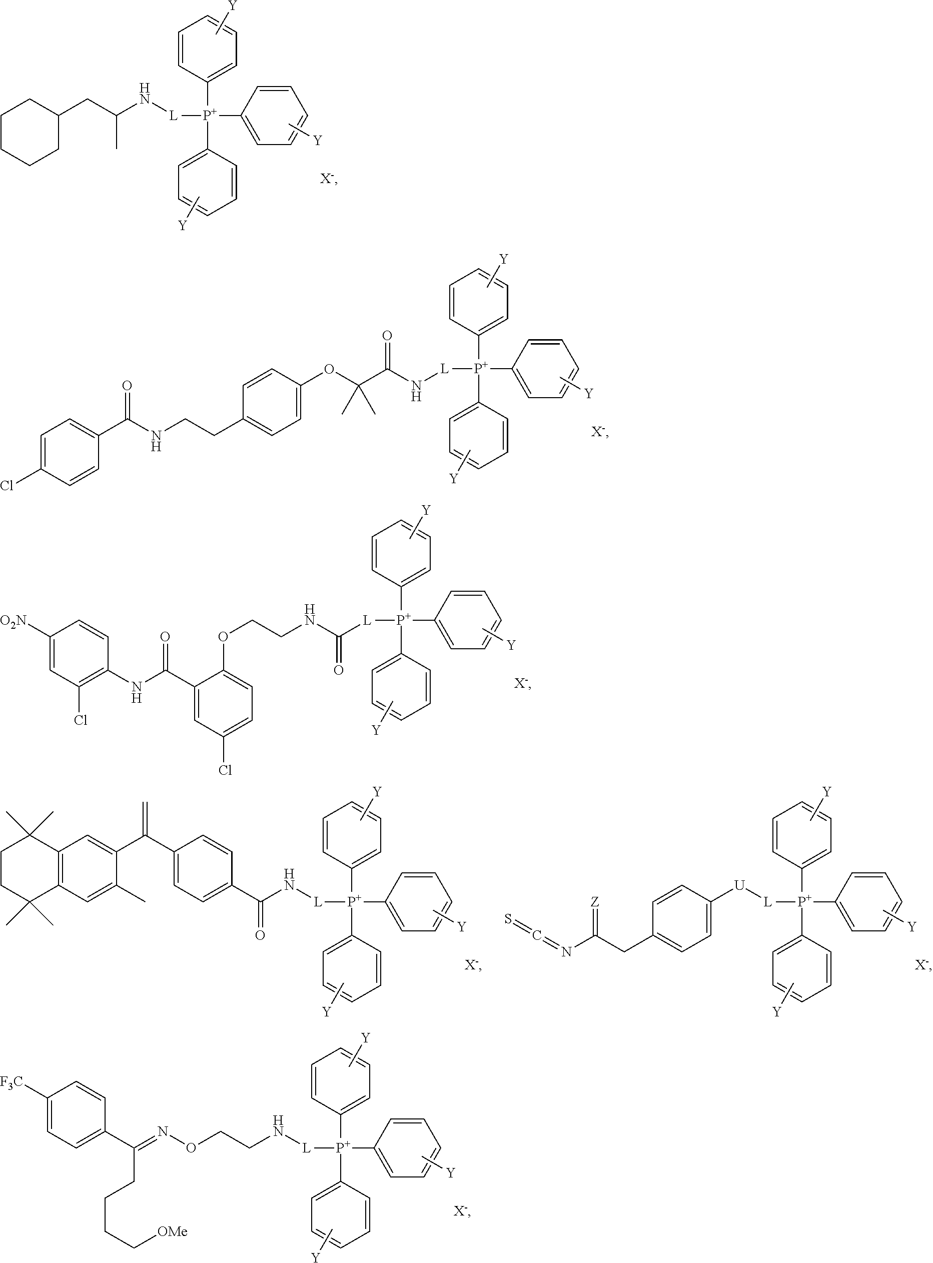

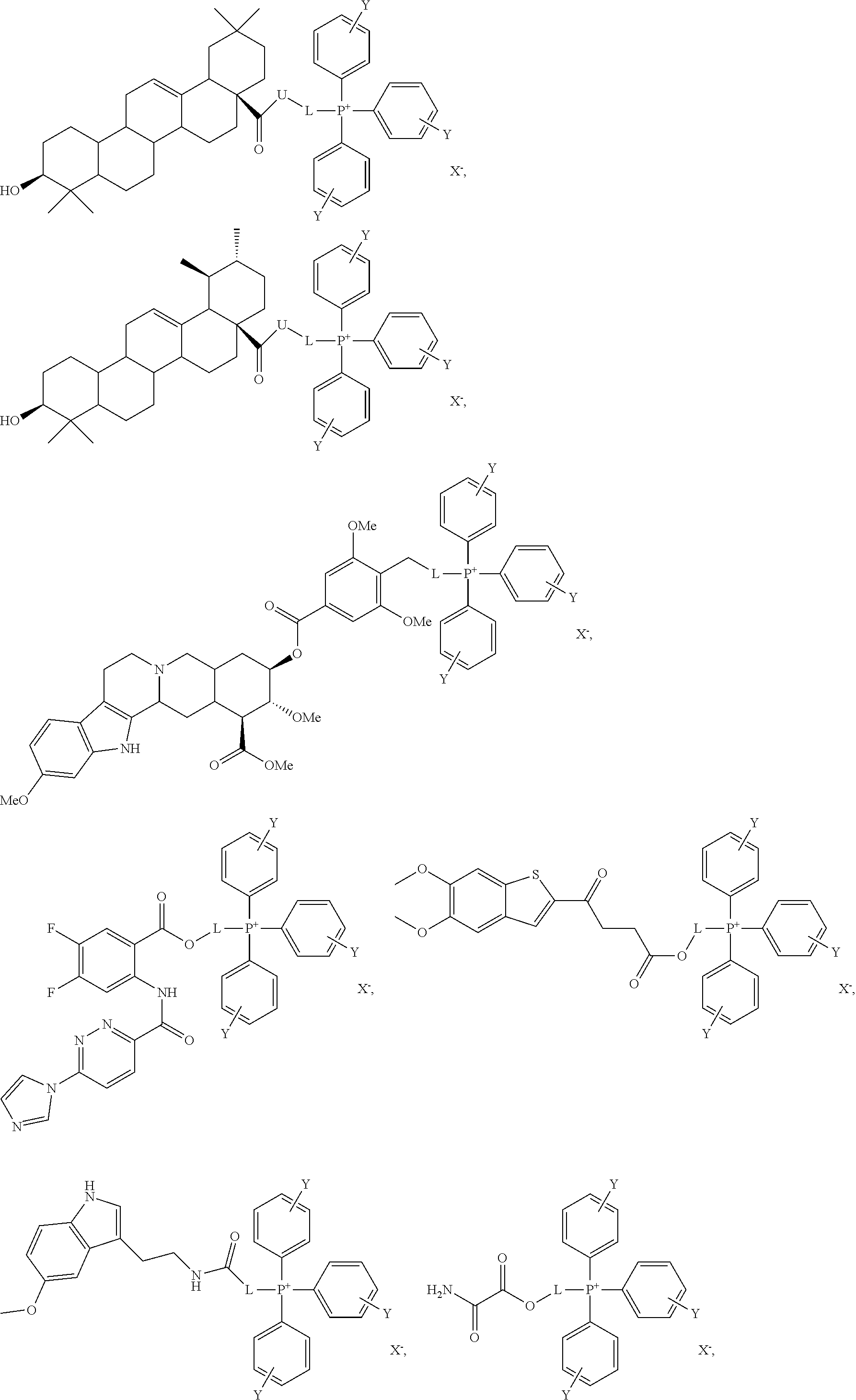

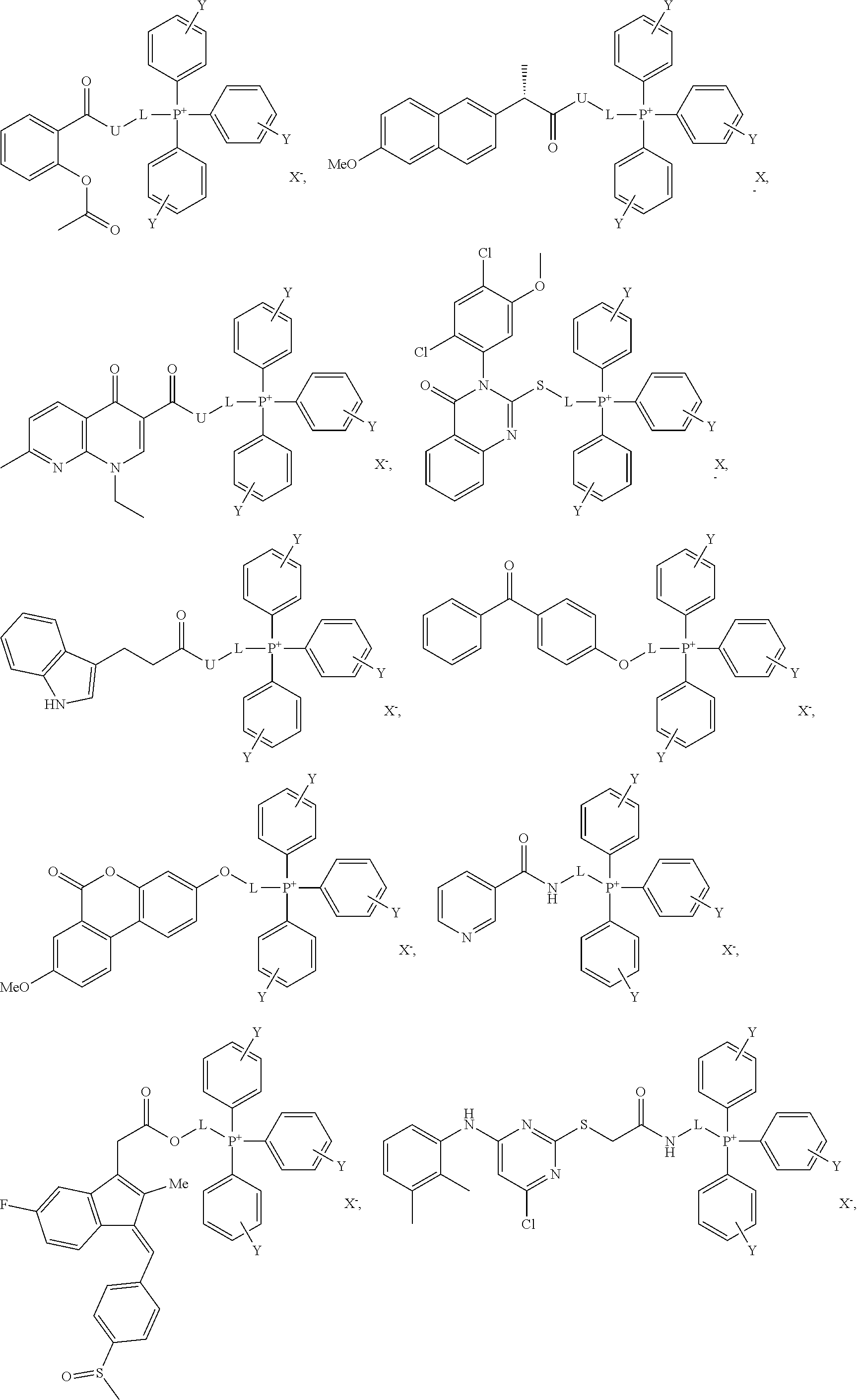

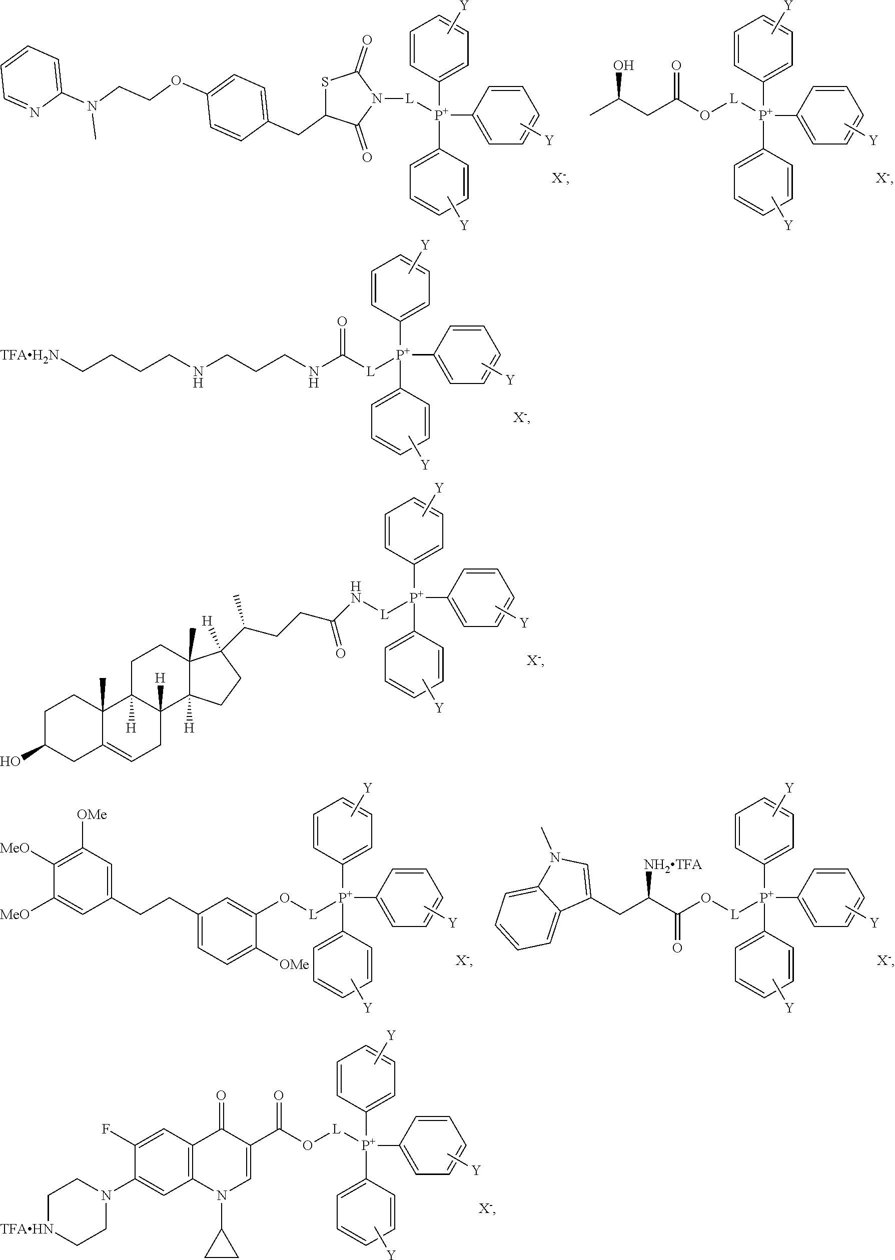

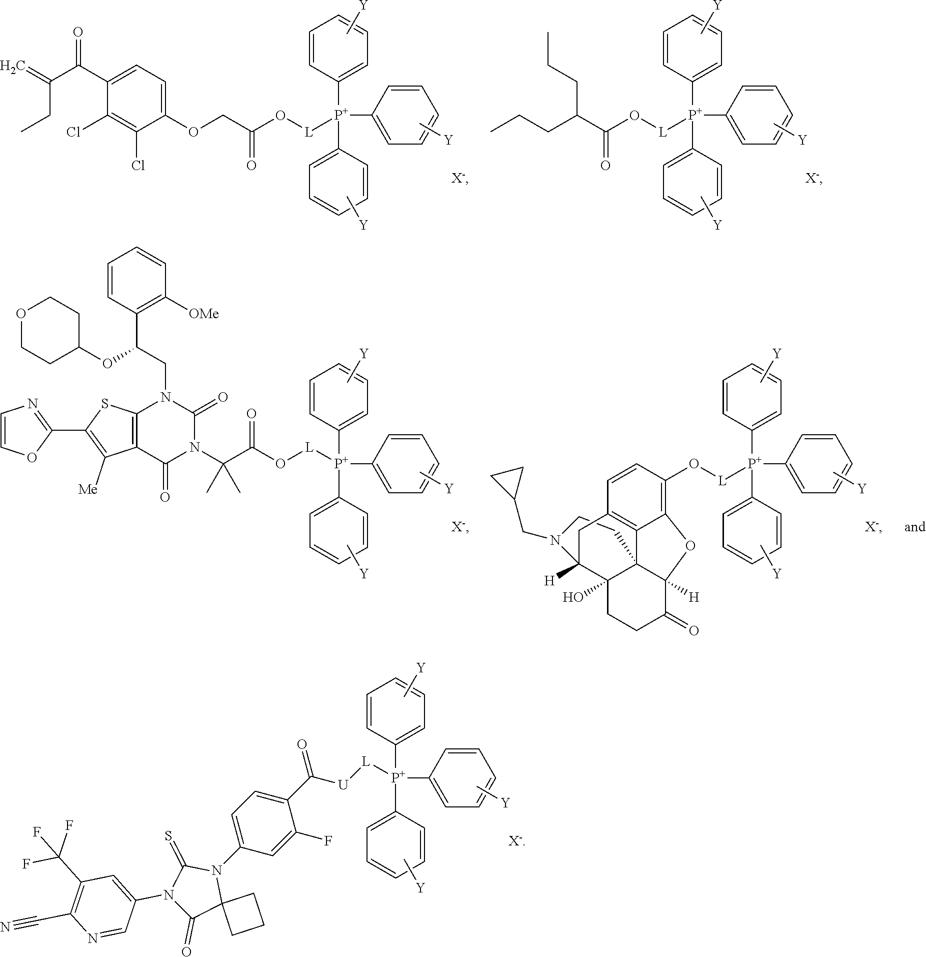

31. The compound of claim 1, wherein the compound is selected from:

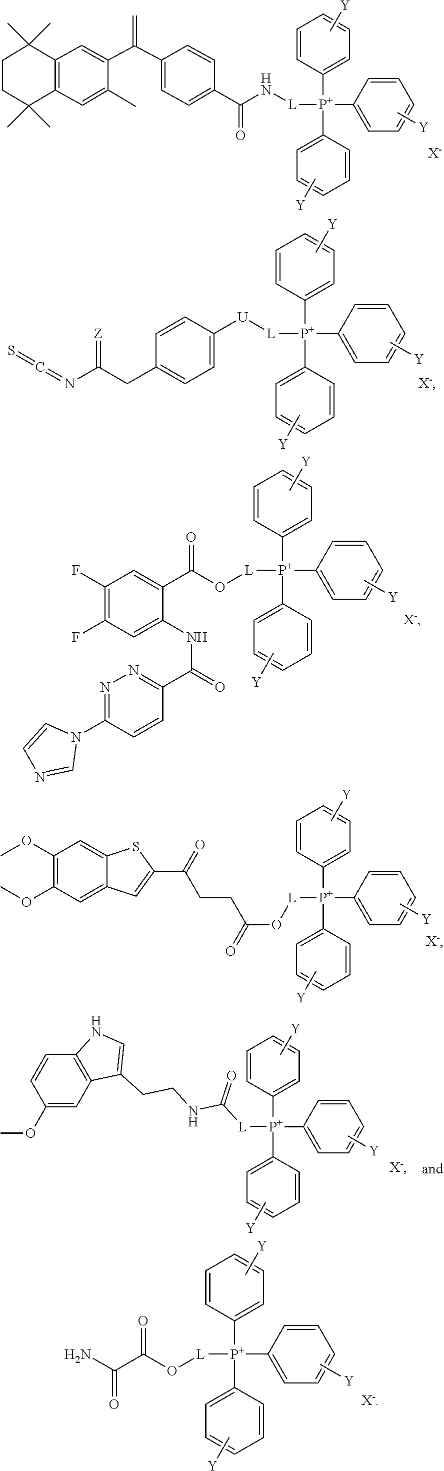

32. The compound of claim 1, wherein the compound is selected from:

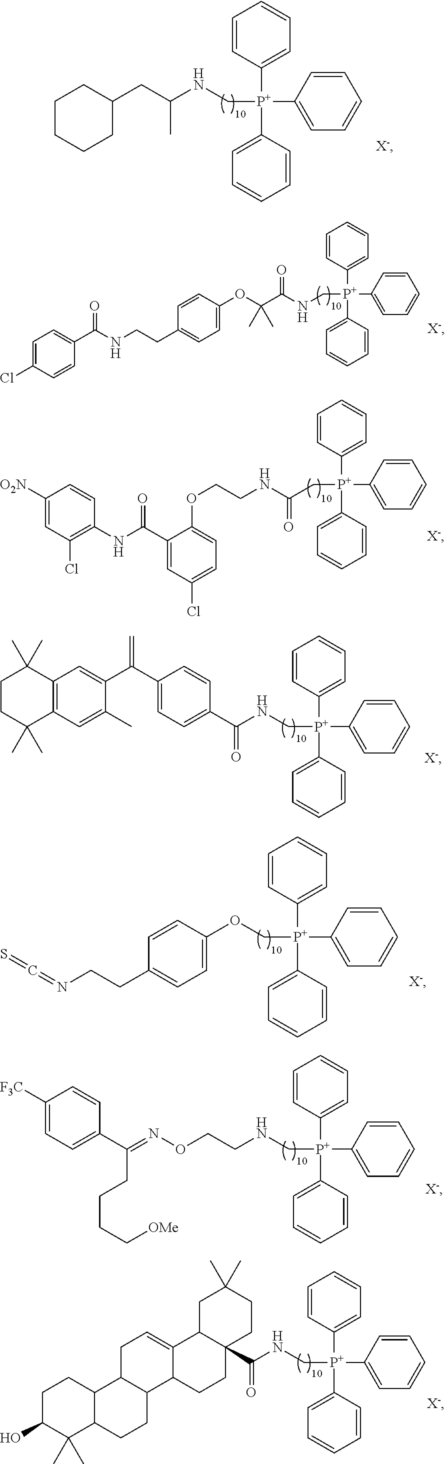

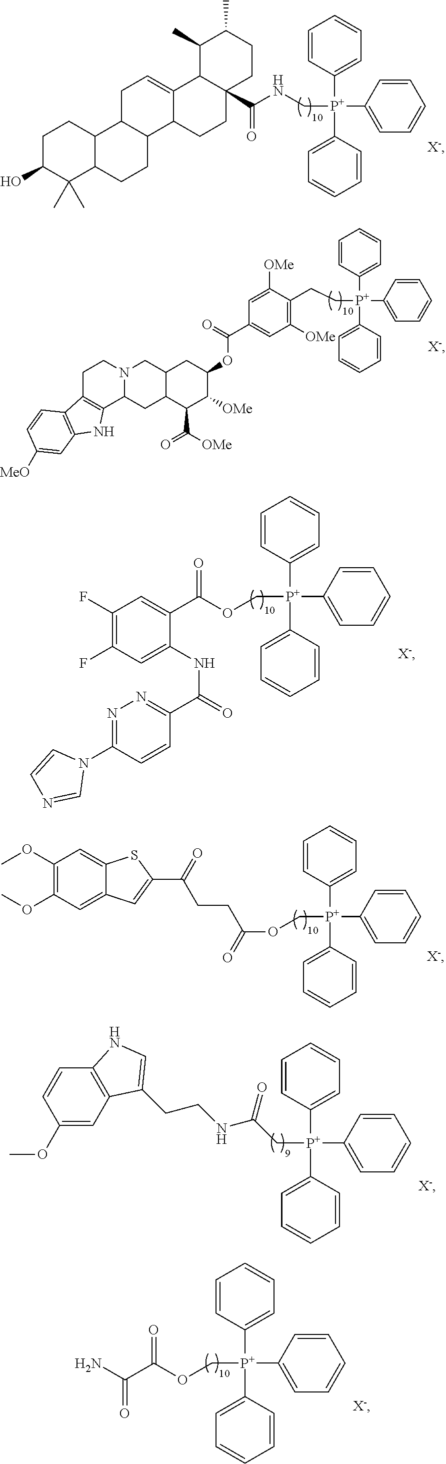

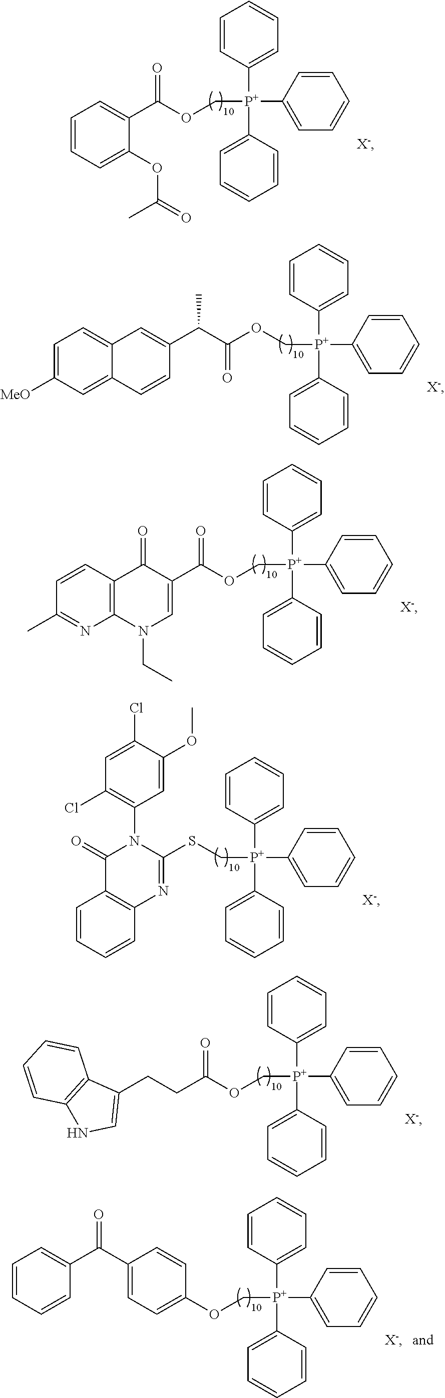

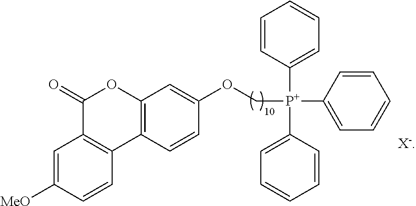

33. The compound of claim 1, wherein the compound is selected from:

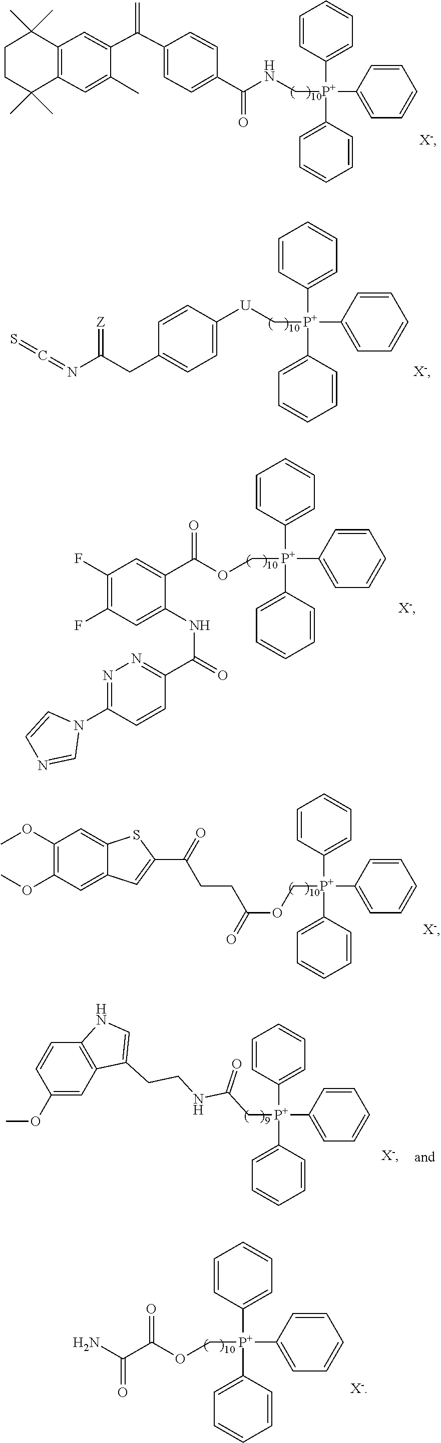

34. The compound of claim 1, wherein the compound is:

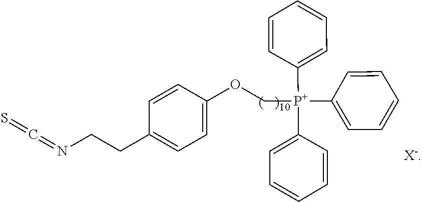

35. A pharmaceutical composition comprising an effective amount of the compound of claim 1 and a pharmaceutically acceptable carrier.

36. A method for treating or preventing cancer, the method comprising administering to a subject in need thereof an effective amount of a compound of claim 1.

37. (canceled)

38. (canceled)
